(19) United States
(12) Patent Application Publication
(10) Pub. No.: US 2026/0250751 A1
(43) Pub. Date: Aug. 27, 2026

(54) METHODS OF DETERMINING GENOMIC COPY NUMBER ALTERATIONS

(71) Applicant: REPARE THERAPEUTICS INC., St-Laurent (CA)

(72) Inventors: Elia Aguado-Fraile, St-Laurent (CA); Adam Petrone, St-Laurent (CA); Jorge Reis-Filho, St-Laurent (CA); Victoria Rimkunas, St-Laurent (CA); Artur Veloso, St-Laurent (CA)

(73) Assignee: REPARE THERAPEUTICS INC., St-Laurent, QC (CA)

(21) Appl. No.: 19/469,929

(22) PCT Filed: Mar. 27, 2024
(86) PCT No.: PCT/CA2024/050384

§ 371 Date: Sep. 26, 2025

Related U.S. Application Data
(60) Provisional application No. 63/455,221, filed on Mar. 28, 2023.

(51) Int. Cl. C12Q 1/6841; A61K 31/437; A61K 31/4985; C12Q 1/6809; C12Q 1/6886

(52) U.S. Cl. CPC C12Q 1/6841; A61K 31/437; A61K 31/4985; C12Q 1/6809; C12Q 1/6886; C12Q 2600/106; C12Q 2600/118; C12Q 2600/166

(57) ABSTRACT

Disclosed herein are methods for identifying a stable control locus for use in a fluorescence in situ hybridization (FISH) assay and for determining whether copy number alterations (e.g., amplification) of a target genomic locus (e.g., a gene, e.g., cyclin E1 (CCNE1)) has occurred in a biological sample (e.g., a non-neoplastic sample, preneoplastic lesion, and/or a tumor). In particular, the disclosure provides an expanded repertoire of genomic loci that can serve as a stable control (e.g., a stable control locus) for FISH assays aimed at determining copy number alterations of a target genomic locus.

### FIELD OF INVENTION

This invention relates to methods of identifying a genomic locus that can serve as control for detecting copy number alterations of a target genomic locus (e.g., a biomarker of cancer, e.g., a gene, e.g., CCNE1) using fluorescence in situ hybridization (FISH).

### BACKGROUND

Fluorescence in situ hybridization (FISH) is a molecular technique commonly used for the detection of cytogenetic abnormalities in a cell, such as the amplification of chromosomes, genetic elements, and/or genes. Since cytogenetic abnormalities are a common characteristic of cancer cells, FISH is often applied for the diagnosis and prognosis of cancer. For example, FISH is commonly used to detect the amplification (e.g., duplication) of cyclin E1 (CCNE1), a hallmark of many carcinomas.

FISH-based methods for detecting amplification or deletion of a gene utilize two fluorescent probes. The first probe (e.g., a target probe) targets a genomic locus (i.e., a target genomic locus) that is of interest to the user while the second probe (e.g., control probe) targets a separate and distinct control locus (i.e., a stable control locus). The ratio of the fluorescent signals produced by the two FISH probes can indicate amplification or deletion of the target genomic locus; therefore, it is important to have a control probe that targets a locus that has a substantially constant (i.e., stable) ploidy across patient samples. However, a stable control locus in the context of cancer cells can vary with different cancer types. Thus, there remains a need in the field for identifying a stable control locus on a cancer-by-cancer basis.

### SUMMARY OF THE INVENTION

In a first aspect, the invention features a method of identifying a stable control locus for use in a fluorescent in situ hybridization (FISH) assay, the method including: (a) determining the ploidy of a plurality of chromosomal bands within a chromosome obtained from a biological sample of a subject; (b) repeating step (a) a plurality of times, wherein the biological sample is obtained from a different subject each time; (c) determining a weighted average (e.g., mean) ploidy of each chromosomal band, wherein a weighted average ploidy between 1.8 copies and 2.2 copies is considered diploid; (d) determining a percentage of subjects that are considered diploid for each chromosomal band; (e) ranking each chromosomal band, from high to low, by the percentage of subjects that are considered diploid; and (f) selecting a chromosomal band within the upper quartile of step (e), thereby identifying the stable control locus.

In some embodiments, the biological sample includes a cancer cell. In some embodiments, the subject is a patient that has been diagnosed with a cancer.

In a second aspect, the invention provides a method of determining if Cyclin E1 (CCNE1) is amplified in a cell, the method including: (a) contacting a population of cells having chromosomal DNA with a plurality of target probes that are capable of emitting a first detectable signal and are complementary to CCNE1, and a plurality of control probes that are capable of emitting a second detectable signal and are complementary to a CCNE1 control locus, wherein the CCNE1 control locus and CCNE1 are on the same chromosomal DNA molecule; (b) quantifying the total number of first detectable signals and second detectable signals in a cell; (c) determining a signal ratio in the cell; (d) repeating step (b) and step (c) a plurality of times, wherein the cell is different each time; (e) determining an average (e.g., mean) signal ratio of step (d); and (f) determining amplification of CCNE1 from the average signal ratio, wherein an average signal ratio ≥2 indicates that CCNE1 is amplified.

In some embodiments, the plurality of target probes binds within the q12 band of chromosome (chr) 19 (chr19 q12), relative to Homo sapiens genome assembly NCBI36.

In some embodiments, the plurality of control probes binds within chr19 q13.43, chr19 q13.41, chr19 q13.42, chr19 p13.3, chr19 q13.32, chr19 q13.33, chr19 q13.31, chr19 p13.13, or chr19 q13.13, relative to Homo sapiens genome assembly NCBI36. In some embodiments, the plurality of control probes binds within chr19 q13.43, relative to Homo sapiens genome assembly NCBI36. In some embodiments, the plurality of control probes binds within nucleotide positions 29,644,930-29,841,126 of chr19 q13.43, relative to Homo sapiens genome assembly NCBI36.

In some embodiments, the contacting occurs in vitro.

In some embodiments, the population of cells are from a human tumor sample.

In some embodiments, the population of cells are from a human subject diagnosed with cancer. In some embodiments, the population of cells are from a human subject at risk of developing cancer. In some embodiments, the population of cells are from a human subject suspected of having cancer.

In some embodiments, the average (e.g., mean) signal ratio is determined from about 20 to about 70 (e.g., 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70) cells. In some embodiments, the average signal ratio is determined from about 50 (e.g., 45, 46, 47, 48, 49, 50, 51, 52, 53, 54, or 55) cells.

In some embodiments, the plurality of target and/or control probes each include a polynucleotide having a modification to a base, sugar, and/or backbone. In some embodiments, the modification is chosen from a 2′-O-methyl (2′-O-Me) modified nucleoside, a phosphorothioate (PS) bond between nucleosides, and a 2′-fluoro (2′-F) modified nucleoside.

In a third aspect, the invention provides a method of determining if a target genomic locus is amplified in a cell, the method including: (a) contacting a population of cells having chromosomal DNA with a plurality of target probes that are capable of emitting a first detectable signal and are complementary to the target genomic locus, and a plurality of control probes that are capable of emitting a second detectable signal and are complementary to a stable control locus, wherein the stable control locus and the target genomic locus are on the same chromosomal DNA molecule; (b) quantifying the total number of first detectable signals and second detectable signals in a cell; (c) determining a signal ratio in the cell; (d) repeating step (b) and step (c) a plurality of times, wherein the cell is different each time; (e) determining an average (e.g., mean) signal ratio of step (d); and (f) determining amplification of the target genomic locus from the average signal ratio, wherein an average signal ratio ≥2 indicates that the target genomic locus is amplified.

In some aspects, the stable control locus is identified by (a) determining the ploidy of a plurality of chromosomal bands within a chromosome obtained from a biological sample of a subject; (b) repeating step (a) a plurality of times, wherein the biological sample is obtained from a different subject each time; (c) determining a weighted average (e.g., mean) ploidy of each chromosomal band, wherein a weighted average ploidy between 1.8 copies and 2.2 copies is considered diploid; (d) determining a percentage of subjects that are considered diploid for each chromosomal band; (e) ranking each chromosomal band, from high to low, by the percentage of subjects that are considered diploid; and (f) selecting a chromosomal band within the upper quartile of step (e), thereby identifying the stable control locus.

In some aspects, the biological sample comprises a cancer cell.

In some embodiments, the contacting occurs in vitro.

In some embodiments, the population of cells are from a human tumor sample.

In some embodiments, the population of cells are from a human subject diagnosed with cancer. In some embodiments, the population of cells are from a human subject at risk of developing cancer. In some embodiments, the population of cells are from a human subject suspected of having cancer.

In some embodiments, the average (e.g., mean) signal ratio is determined from about 30 to about 70 (e.g., 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70) cells. In some embodiments, the average signal ratio is determined from about 50 (e.g., 45, 46, 47, 48, 49, 50, 51, 52, 53, 54, or 55) cells.

In some embodiments, the plurality of target and/or control probes each include a polynucleotide having a modification to a base, sugar, and/or backbone. In some embodiments, the modification is chosen from a 2′-O-methyl (2′-O-Me) modified nucleoside, a phosphorothioate (PS) bond between nucleosides, and a 2′-fluoro (2′-F) modified nucleoside.

In a fourth aspect, the invention features a method of identifying a patient at risk of having cancer or suspected of having cancer, the method including: (a) obtaining a sample of cells from a subject; and (b) performing the method of the second aspect, wherein the subject is identified as a patient if the CCNE1 gene is determined to be amplified in the sample.

In a fifth aspect, the invention features a method of treating the patient identified in the first or fourth aspect (e.g., a patient at risk of having cancer, suspected of having cancer, or diagnosed with cancer) the method including administering an effective amount of an inhibitor of a membrane-associated tyrosine and threonine-specific cdc2-inhibitory kinase (Myt1).

In some embodiments, the Myt1 inhibitor is a compound of formula (I):

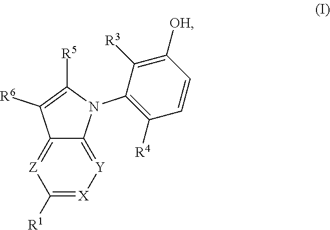

or a pharmaceutically acceptable salt thereof,
wherein
   each of X, Y, and Z is independently N or CR²;
   R¹ and each R² are independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B}; or R¹ combines with one R² that is vicinal to R¹ to form an optionally substituted C₃₋₆ alkylene;
   each of R³ and R⁴ is independently optionally substituted C₁₋₆ alkyl or halogen;
   R⁵ is H or —N(R⁷)₂;
   R⁶ is —C(O)NH(R⁸), —C(O)R^{7A}, or —SO₂R^{7A};
   each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;
   each R^{7A} is independently optionally substituted C₁₋₆ alkyl, optionally substituted C₃₋₈ cycloalkyl, or optionally substituted C₆₋₁₀ aryl;
   each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;
   each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;
   Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene.

In some embodiments, the Myt1 inhibitor is enriched for the atropisomer of formula (IA):

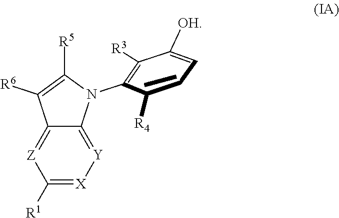

In some embodiments, X is CR². In some embodiments, the Myt1 inhibitor is of formula (II):

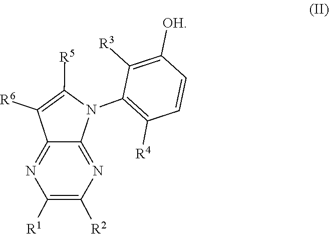

In some embodiments, the Myt1 inhibitor is enriched for the atropisomer of formula (IIA):

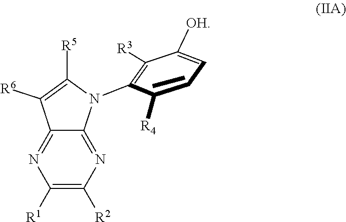

In some embodiments, the Myt1 inhibitor is of formula (III):

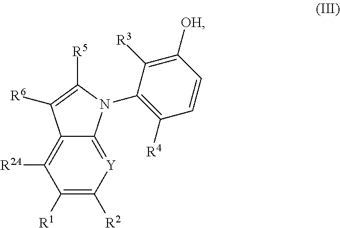

wherein R^{2A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁷)₂, —OR⁷, —C(O)N(R⁸)₂, —SO₂N(R⁸)₂, —SO₂R^{7A}, or -Q-R^{7B}.

In some embodiments, the Myt1 inhibitor is enriched for the atropisomer of formula (IIIA):

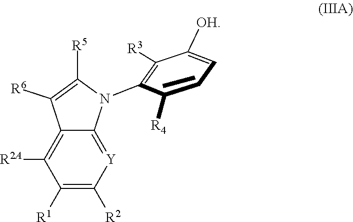

In some embodiments, R^{2A} is hydrogen, optionally substituted C₁₋₆ alkyl, or halogen.

In some embodiments, R³ is optionally substituted C₁₋₆ alkyl. In some embodiments, R³ is halogen. In some embodiments, R⁴ is optionally substituted C₁₋₆ alkyl. In some embodiments, R⁴ is halogen (e.g., chlorine).

In some embodiments, R² is hydrogen. In some embodiments, R² is optionally substituted C₁₋₆ alkyl. In some embodiments, R² is optionally substituted methyl or optionally substituted isopropyl. R² is halogen.

In some embodiments, R¹ is hydrogen. In some embodiments, R¹ is halogen. In some embodiments, R¹ is chlorine or bromine. In some embodiments, R¹ is optionally substituted C₁₋₆ alkyl. In some embodiments, R¹ is optionally substituted methyl, optionally substituted ethyl, optionally substituted isopropyl, or optionally substituted butyl. In some embodiments, R¹ is optionally substituted C₁₋₉ heteroaryl. In some embodiments, R¹ is 1,3-thiazolyl, 1,2-thiazolyl, 1,3-oxazolyl, benzo-1,3-thiazolyl, benzo-1,3-oxazolyl, indolyl, benzimidazolyl, pyridyl, imidazolyl, pyrimidyl, pyrazinyl, pyridazinyl, or pyrazolyl, wherein R¹ is optionally substituted with substituents as defined for optionally substituted C₁₋₉ heteroaryl. In some embodiments, R¹ is optionally substituted C₃₋₈ cycloalkyl. In some embodiments, R¹ is cyclopropyl, cyclobutyl, cyclopentyl, or cyclohexyl, wherein R¹ is optionally substituted with substituents as defined for optionally substituted C₃₋₈ cycloalkyl. In some embodiments, R¹ is optionally substituted C₂₋₉ heterocyclyl. In some embodiments, R¹ is 1,2,3,6-tetrahydropyridinyl, piperidinyl, morpholinyl, piperazinyl, thiomorpholinyl, oxa-aza-spiro[3,3]heptane, or oxa-aza-bicyclo[3.2.1]octane, wherein R¹ is optionally substituted with substituents as defined for optionally substituted C₂₋₉ heterocyclyl. In some embodiments, R¹ is optionally substituted C₃₋₈ cycloalkyl. In some embodiments, R¹ is optionally substituted cyclohexenyl or optionally substituted cyclopentenyl. In some embodiments, R¹ is optionally substituted C₆₋₁₀ aryl. In some embodiments, R¹ is optionally substituted phenyl.

In some embodiments, R¹ is -Q-R^{7B}. In some embodiments, Q is optionally substituted C₂₋₆ alkynylene. In some embodiments, Q is optionally substituted C₁₋₆ alkylene. In some embodiments, Q is optionally substituted C₆₋₁₀ arylene. In some embodiments, R^{7B} is optionally substituted C₂₋₉ heterocyclyl. In some embodiments, R^{7B} is optionally substituted C₆₋₁₀ aryl.

In some embodiments, R¹ is optionally substituted with one, two, or three groups independently selected from the group consisting of methyl, difluoromethyl, trifluoromethyl, fluorine, chlorine, bromine, amino, hydroxyl, cyano, oxo, —C(O)NH₂, —C(O)NH(Me), —C(O)N(Me)₂, —(CH₂)ₙ—C(O)OH, and —(CH₂)ₙ—C(O)Ot-Bu, wherein n is 0 or 1.

In some embodiments, R¹ is —N(R⁷)₂. In some embodiments, R¹ is diethylamino.

In some embodiments, R⁵ is hydrogen. In some embodiments, R⁵ is —N(R⁷)₂. In some embodiments, R⁵ is —NH₂. In some embodiments, R⁶ is —C(O)NH(R⁶). In some embodiments, R⁶ is —C(O)NH₂. In some embodiments, R⁶ is —C(O)NH(Me). In some embodiments, R⁶ is —SO₂R^{7A}. In some embodiments, R⁶ is —SO₂Me.

In some embodiments, the Myt1 inhibitor is selected from the group consisting of compounds 1-328 of Table 1 and pharmaceutically acceptable salts thereof. In some embodiments, the Myt1 inhibitor is compound 182 of Table 1.

### Definitions

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Unless otherwise stated, nucleic acid sequences described herein are given, when read from left to right, in the 5′ to 3′ direction. Nucleic acid sequences may be provided as DNA, as RNA, or a combination of DNA and RNA (e.g., a chimeric nucleic acid).

The term “comprise” is intended to mean “include”. The use of the term “including,” as well as other forms, such as “includes” and “included,” is not limiting. Where a term is provided in the singular, it also contemplates aspects of the invention described by the plural of that term. The term “and/or” where used herein is to be taken as specific disclosure of each of the multiple specified features or components with or without another. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone). Other terms and definitions are defined below:

The term “about,” as applied to one or more values of interest, refers to a value that falls within 10% in either direction (greater than or less than) of a stated reference value, unless otherwise stated or otherwise evident from the context (except where such number would exceed 100% of a possible value).

The term “acyl,” as used herein, represents a group —C(═O)—R, where R is alkyl, alkenyl, alkynyl, cycloalkyl, cycloalkenyl, cycloalkynyl, aryl, heteroaryl, or heterocyclyl. Acyl may be optionally substituted as described herein for each respective R group.

The term “alkanoyl,” as used herein, represents a hydrogen or an alkyl group that is attached to the parent molecular group through a carbonyl group and is exemplified by formyl (i.e., a carboxyaldehyde group), acetyl, propionyl, butyryl, and iso-butyryl. Unsubstituted alkanoyl groups contain from 1 to 7 carbons. The alkanoyl group may be unsubstituted of substituted (e.g., optionally substituted C₁₋₇ alkanoyl) as described herein for alkyl group. The ending “-oyl” may be added to another group defined herein, e.g., aryl, cycloalkyl, and heterocyclyl, to define “aryloyl,” “cycloalkanoyl,” and “(heterocyclyl)oyl.” These groups represent a carbonyl group substituted by aryl, cycloalkyl, or heterocyclyl, respectively. Each of “aryloyl,” “cycloalkanoyl,” and “(heterocyclyl)oyl” may be optionally substituted as defined for “aryl,” “cycloalkyl,” or “heterocyclyl,” respectively.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkenylene,” as used herein, refers to a divalent alkenyl group. An optionally substituted alkenylene is an alkenylene that is optionally substituted as described herein for alkenyl.

The term “alkoxy,” as used herein, represents a chemical substituent of formula —OR, where R is a C₁₋₆ alkyl group, unless otherwise specified. In some embodiments, the alkyl group can be further substituted as defined herein. The term “alkoxy” can be combined with other terms defined herein, e.g., aryl, cycloalkyl, or heterocyclyl, to define an “aryl alkoxy,” “cycloalkyl alkoxy,” and “(heterocyclyl)alkoxy” groups. These groups represent an alkoxy that is substituted by aryl, cycloalkyl, or heterocyclyl, respectively. Each of “aryl alkoxy,” “cycloalkyl alkoxy,” and “(heterocyclyl)alkoxy” may optionally substituted as defined herein for each individual portion.

The term “alkoxyalkyl,” as used herein, represents a chemical substituent of formula -L-O—R, where L is C₁₋₆ alkylene, and R is C₁₋₆ alkyl. An optionally substituted alkoxyalkyl is an alkoxyalkyl that is optionally substituted as described herein for alkyl.

The term “alkyl,” as used herein, refers to an acyclic straight or branched chain saturated hydrocarbon group, which, when unsubstituted, has from 1 to 12 carbons, unless otherwise specified.

In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: amino; alkoxy; aryl; aryloxy; azido; cycloalkyl; cycloalkoxy; cycloalkenyl; cycloalkynyl; halo; heterocyclyl; (heterocyclyl)oxy; heteroaryl; hydroxy; nitro; thiol; silyl; cyano; alkylsulfonyl; alkylsulfinyl; alkylsulfenyl; ═O; ═S; —C(O)R or —SO₂R, where R is amino; and ═NR′, where R′ is H, alkyl, aryl, or heterocyclyl. Each of the substituents may itself be unsubstituted or, valency permitting, substituted with unsubstituted substituent(s) defined herein for each respective group.

The term “alkylene,” as used herein, refers to a divalent alkyl group. An optionally substituted alkylene is an alkylene that is optionally substituted as described herein for alkyl.

The term “alkylamino,” as used herein, refers to a group having the formula —N(R^{N1})₂ or —NHR^{N1}, in which R^{N1} is alkyl, as defined herein. The alkyl portion of alkylamino can be optionally substituted as defined for alkyl. Each optional substituent on the substituted alkylamino may itself be unsubstituted or, valency permitting, substituted with unsubstituted substituent(s) defined herein for each respective group.

The term “alkylsulfenyl,” as used herein, represents a group of formula —S-(alkyl). Alkylsulfenyl may be optionally substituted as defined for alkyl.

The term “alkylsulfinyl,” as used herein, represents a group of formula —S(O)-(alkyl). Alkylsulfinyl may be optionally substituted as defined for alkyl.

The term “alkylsulfonyl,” as used herein, represents a group of formula —S(O)₂-(alkyl). Alkylsulfonyl may be optionally substituted as defined for alkyl.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. The alkynyl groups may be unsubstituted or substituted (e.g., optionally substituted alkynyl) as defined for alkyl.

The term “alkynylene,” as used herein, refers to a divalent alkynyl group. An optionally substituted alkynylene is an alkynylene that is optionally substituted as described herein for alkynyl.

The term “amino,” as used herein, represents —N(R^{N1})₂, where, if amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —C(O)OR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., —NHR^{N1}), where R^{N1} is independently —OH, SO₂OR^{N2}, —SO₂R^{N2}, —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In some embodiments, an amino group is —NHR^{N1}, in which R^{N1} is optionally substituted alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; —(CH₂)ₙ—C(O)OR^{A}; —C(O)R; and —SO₂R, where R is amino or alkyl, R^{A} is H or alkyl, and n is 0 or 1. Each of the substituents may itself be unsubstituted or substituted with unsubstituted substituent(s) defined herein for each respective group.

The term “arylene,” as used herein, refers to a divalent aryl group. An optionally substituted arylene is an arylene that is optionally substituted as described herein for aryl.

The term “aryloxy,” as used herein, represents a chemical substituent of formula —OR, where R is an aryl group, unless otherwise specified. In optionally substituted aryloxy, the aryl group is optionally substituted as described herein for aryl.

The term “azido,” as used herein, represents an —N₃ group.

The terms “cancer” and “cancerous” refer to or describe the physiological condition in mammals that is typically characterized by unregulated cell growth. Cancers include solid tumor cancers and non-solid tumor cancers and locally advanced or metastatic cancers (e.g., locally advanced or metastatic tumors). Examples of cancer are carcinoma, lymphoma, blastoma, sarcoma, glioma, mesothelioma, and leukemia or lymphoid malignancies. Particular examples of cancers are urothelial carcinoma (UC), including locally advanced and metastatic UC (mUC), bladder cancer (e.g., muscle invasive bladder cancer (MIBC) and non-muscle invasive bladder cancer (NMIBC), e.g., BCG-refractory NMIBC), MIBC urothelial bladder cancer (UBC); kidney or renal cancer (e.g., renal cell carcinoma (RCC)); cancer of the urinary tract; lung cancer, such as small cell lung cancer (SCLC), which includes extensive stage SCLC (ES-SCLC); non-small cell lung cancer (NSCLC), which includes squamous NSCLC or non-squamous NSCLC, including locally advanced unresectable NSCLC (e.g., Stage IIIB NSCLC), or recurrent or metastatic NSCLC (e.g., Stage IV NSCLC), adenocarcinoma of the lung, or squamous cell cancer (e.g., epithelial squamous cell cancer (e.g., squamous carcinoma of the lung); pancreatic cancer (e.g., pancreatic ductal adenocarcinoma (PDAC), e.g., metastatic PDAC)); head and neck cancer (e.g., SCCHN, e.g., recurrent/metastatic PD-L1-positive SCCHN, and head and neck squamous cell cancer (HNSCC); ovarian cancer (OC); esophageal cancer; cancer of the peritoneum; hepatocellular cancer; gastric cancer (GC) (e.g., gastroesophageal junction (GEJ) cancer) and stomach cancer, including gastrointestinal cancer and gastrointestinal stromal cancer; glioblastoma; cancer of the urinary tract; hepatoma; breast cancer (e.g., HER2+ breast cancer and triple-negative breast cancer (TNBC (e.g., early TNBC (eTNBC)), which are estrogen receptors (ER−), progesterone receptors (PgR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer (e.g., pancreatic ductal adenocarcinoma (PDAC)); glioblastoma; cervical cancer (e.g., a Stage IVB, metastatic, recurrent, or persistent cervical cancer, e.g., a metastatic and/or recurrent PD-L1-positive cervical carcinoma); ovarian cancer; liver cancer (e.g., hepatocellular carcinoma (HCC), e.g., locally advanced or metastatic HCC and/or unresectable HCC); hepatoma; colon cancer; rectal cancer; colorectal cancer (CRC; e.g., CRC with microsatellite-stable (MSS) and microsatellite instability (MSI) low (MSI-Low)); endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL)); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myelogenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

The term “carbocyclic,” as used herein, represents an optionally substituted C₃₋₁₆ monocyclic, bicyclic, or tricyclic structure in which the rings, which may be aromatic or non-aromatic, are formed by carbon atoms. Carbocyclic structures include cycloalkyl, cycloalkenyl, cycloalkynyl, and certain aryl groups.

The term “carbonyl,” as used herein, represents a —C(O)— group.

The terms “CCNE1” and “cyclin E1,” as used interchangeably herein, refer to G1/S specific cyclin E1 (Gene name: CCNE1).

As used herein, a “CCNE control locus” is any one of the following loci: chr19 q13.43, chr19 q13.41, chr19 q13.42, chr19 p13.3, chr19 q13.32, chr19 q13.33, chr19 q13.31, chr19 p13.13, or chr19 q13.13. A CCNE1 control locus described herein can be used in a fluorescent in situ hybridization (FISH) assay when a target genomic locus is, or includes, chr19 q12 or the cyclin E1 (CCNE1) gene.

The term “cyano,” as used herein, represents —CN group.

The term “cycloalkenyl,” as used herein, refers to a non-aromatic carbocyclic group having at least one double bond in the ring and from three to ten carbons (e.g., a C₃₋₁₀ cycloalkenyl), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenyl alkyl,” as used herein, represents an alkyl group substituted with a cycloalkenyl group, each as defined herein. The cycloalkenyl and alkyl portions may be substituted as the individual groups defined herein.

The term “cycloalkenylene,” as used herein, represents a divalent cycloalkenyl group. An optionally substituted cycloalkenylene is a cycloalkenylene that is optionally substituted as described herein for cycloalkyl.

The term “cycloalkoxy,” as used herein, represents a chemical substituent of formula —OR, where R is cycloalkyl group, unless otherwise specified. In some embodiments, the cycloalkyl group can be further substituted as defined herein.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; heteroaryl; hydroxy; nitro; thiol; silyl; cyano; ═O; ═S; —SO₂R, where R is optionally substituted amino; ═NR′, where R′ is H, alkyl, aryl, or heterocyclyl; and —CON(R^{A})₂, where each R^{A} is independently H or alkyl, or both R^{A}, together with the atom to which they are attached, combine to form heterocyclyl. Each of the substituents may itself be unsubstituted or substituted with unsubstituted substituent(s) defined herein for each respective group.

The term “cycloalkyl alkyl,” as used herein, represents an alkyl group substituted with a cycloalkyl group, each as defined herein. The cycloalkyl and alkyl portions may be optionally substituted as the individual groups described herein.

The term “cycloalkylene,” as used herein, represents a divalent cycloalkyl group. An optionally substituted cycloalkylene is a cycloalkylene that is optionally substituted as described herein for cycloalkyl.

The term “cycloalkynyl,” as used herein, refers to a monovalent carbocyclic group having one or two carbon-carbon triple bonds and having from eight to twelve carbons, unless otherwise specified. Cycloalkynyl may include one transannular bond or bridge. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as defined for cycloalkyl.

The term “cytogenetic abnormality” is used herein to refer to chromosomal abnormalities or chromosomal defects, such as those arising from chromosomal deletion, duplication, nondisjunction, inversion, amplification and translocation. Abnormalities arising from chromosomal deletion, duplication, amplification and nondisjunction events can result in viable cells with aneuploidy. The term “cytogenetic abnormality” used herein can also refer to abnormalities or defects within a chromosome, such the deletion, duplication, or amplification of genes, loci, genetic elements, or bands within the chromosome (e.g., chromosomal bands).

The terms “diagnosed” and “diagnosis” are used herein to refer to the identification or classification of a molecular or pathological state, disease or condition (e.g., cancer). For example, “diagnosed” may refer to identification of a particular type of cancer.

“Disease” or “condition” refer to a state of being or health status of a patient or subject capable of being treated with the compounds or methods provided herein.

The term “target genomic locus” is used herein to refer to a polynucleotide sequence (e.g., one or more genes, or a chromosomal band) within one or more chromosomal DNA molecules (e.g., nuclear and/or mitochondrial DNA) and has at least 80%, at least 90%, at least 95%, at least 96%, at least 97%, at least 98%, at least 99%, or 100% complementarity to the target probe utilized in the methods described herein. For example, the target genomic locus may be the CCNE1 gene, a region within the CCNE1 gene, a region including the CCNE1 gene, chr19 q12, or a region within chr19 q12.

The term “halo,” as used herein, represents a halogen selected from bromine, chlorine, iodine, and fluorine.

As used herein, the terms “human epidermal growth factor receptor 2” and “HER2” are used to refer to the erb-b2 receptor tyrosine kinase 2 (ERBB2) gene (e.g., NCBI Gene ID: 2064) or an encoded transcript (e.g., GenBank: MW358920.1 or LN812234.1; or NCBI Reference Sequences: NM_001382796.1, NM_001382800.1, NM_001382790.1, NM_001382803.1, or NM_001382805.1) or protein product thereof (e.g., UniProtKB/Swiss-Prot: P04626.1; NCBI Reference Sequences: NP_001369725.1, NP_001369719.1, NP_001369728.1, or NP_001369731.1; or GenBank: QSQ86066.1).

As used herein, the term “HER2+” or “HER2-positive” is used to refer to a cell that overexpresses HER2 mRNA and/or protein at increased levels relative to a control (e.g., a non-cancerous cell). Generally, immunohistochemistry (IHC) or FISH assay can be utilized to identify a HER2+ cell.

The term “heteroalkyl,” as used herein refers to an alkyl, alkenyl, or alkynyl group interrupted once by one or two heteroatoms; twice, each time, independently, by one or two heteroatoms; three times, each time, independently, by one or two heteroatoms; or four times, each time, independently, by one or two heteroatoms. Each heteroatom is, independently, O, N, or S. In some embodiments, the heteroatom is O or N. None of the heteroalkyl groups includes two contiguous oxygen or sulfur atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteroatom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, alkenyl, alkynyl, aryl, cycloalkyl, cycloalkenyl, cycloalkynyl, heterocyclyl, or cyano, where each R^{N2} is independently H, alkyl, cycloalkyl, cycloalkenyl, cycloalkynyl, aryl, or heterocyclyl, and each R^{N3} is independently alkyl, cycloalkyl, cycloalkenyl, cycloalkynyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group.

The term “heteroarylene,” as used herein, represents a divalent heteroaryl. An optionally substituted heteroarylene is a heteroarylene that is optionally substituted as described herein for heteroaryl.

The term “heterocyclyl,” as used herein, represents a monocyclic, bicyclic, tricyclic, or tetracyclic ring system having fused, bridging, and/or spiro 3-, 4-, 5-, 6-, 7-, or 8-membered rings, unless otherwise specified, containing one, two, three, or four heteroatoms independently selected from the group consisting of nitrogen, oxygen, and sulfur. In some embodiments, “heterocyclyl” is a monocyclic, bicyclic, tricyclic, or tetracyclic ring system having fused or bridging 5-, 6-, 7-, or 8-membered rings, unless otherwise specified, containing one, two, three, or four heteroatoms independently selected from the group consisting of nitrogen, oxygen, and sulfur. Heterocyclyl can be aromatic or non-aromatic. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups include from 1 to 16 carbon atoms unless otherwise specified. Certain heterocyclyl groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; —C(O)R or —SO₂R, where R is amino or alkyl; ═O; ═S; ═NR′, where R′ is H, alkyl, aryl, or heterocyclyl. Each of the substituents may itself be unsubstituted or substituted with unsubstituted substituent(s) defined herein for each respective group.

The term “heterocyclylene,” as used herein, represents a divalent heterocyclyl. An optionally substituted heterocyclylene is a heterocyclylene that is optionally substituted as described herein for heterocyclyl.

The term “(heterocyclyl)oxy,” as used herein, represents a chemical substituent of formula —OR, where R is a heterocyclyl group, unless otherwise specified. (Heterocyclyl)oxy can be optionally substituted in a manner described for heterocyclyl.

The terms “hydroxyl” and “hydroxy,” as used interchangeably herein, represent an —OH group.

“Homo sapiens genome assembly” is used herein to refer to the National Center for Biotechnology Information's (NCBI) full human genome assembly version 36, commonly known as “NCBI36”, which can be found at: www.ncbi.nlm.nih.gov/assembly/GCF 000001405.12/. The NCBI36 Homo sapiens genome assembly is synonymous with the University of California, Santa Cruz's (UCSC) Human Genome (hg) assembly version 18, commonly known as “hg18”.

As used herein, “hybridization” or “annealing” of nucleic acids is achieved when one or more nucleoside residues within a polynucleotide base pair with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The term “Myt1,” as used herein, refers to membrane-associated tyrosine and threonine-specific cdc2-inhibitory kinase (Myt1) (Gene name PKMYT1).

The term “Myt1 inhibitor,” as used herein, represents a compound that upon contacting the enzyme Myt1, whether in vitro, in cell culture, or in an animal, reduces the activity of Myt1, such that the measured Myt1 IC₅₀ is 10 μM or less (e.g., 5 μM or less or 1 μM or less). For certain Myt1 inhibitors, the Myt1 IC₅₀ may be 100 nM or less (e.g., 10 nM or less, or 3 nM or less) and could be as low as 100 μM or 10 μM. Preferably, the Myt1 IC₅₀ is 1 nM to 1 μM (e.g., 1 nM to 750 nM, 1 nM to 500 nM, or 1 nM to 250 nM). Even more preferably, the Myt1 IC₅₀ is less than 20 nm (e.g., 1 nM to 20 nM).

The term “nitro,” as used herein, represents an —NO₂ group.

The term “oxo,” as used herein, represents a divalent oxygen atom (e.g., the structure of oxo may be shown as ═O).

As used herein, the terms “programmed death ligand 1” and “PD-L1” are used to refer to the CD274 gene (e.g., NCBI Gene ID: 29126) or an encoded transcript (e.g., NCBI Reference Sequences: NR_052005.2, NM_014143.4, NM_001267706.2, XM_047423262.1, or NM_001314029.2) or protein product thereof (e.g., GenBank: AA113735.1, AA113737.1, AAH74984.1, KA14006593.1; or NCBI Reference Sequence: NP_001300958.1). Encoded PD-L1 proteins function as an immune inhibitory receptor ligand and are commonly expressed in hematopoietic and non-hematopoietic cells, such as T cells and B cells and various types of tumor cells.

As used herein, the term “PD-L1+” or “PD-L1-positive” is used to refer to a cell that overexpresses PD-L1 mRNA and/or protein at increased levels relative to a control (e.g., a non-cancerous cell). Generally, immunohistochemistry (IHC) or FISH assay can be utilized to identify a PD-L1+ cell.

The term “Ph,” as used herein, represents phenyl.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

“Polynucleotide”, “oligonucleotide”, or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The terms “polynucleotide” and “nucleic acid” specifically includes mRNA and cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, aspects wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides, oligonucleotides, and nucleic acids referred to herein, including RNA and DNA.

The term “protecting group,” as used herein, represents a group intended to protect a hydroxy, an amino, or a carbonyl from participating in one or more undesirable reactions during chemical synthesis. The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino, amido, heterocyclic N—H, or hydrazine) group from participating in one or more undesirable reactions during chemical synthesis. Commonly used O- and N-protecting groups are disclosed in Greene, “Protective Groups in Organic Synthesis,” 3rd Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p methoxybenzyloxycarbonyl, p-nitrobenzyloxycarbonyl, 2-nitrobenzyloxycarbonyl, p bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, α,α-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, p-methoxybenzyl, 2,4-dimethoxybenzyl, triphenylmethyl, benzyloxymethyl, and the like, silylalkylacetal groups such as [2-(trimethylsilyl)ethoxy]methyl and silyl groups such as trimethylsilyl, and the like. Useful N-protecting groups are formyl, acetyl, benzoyl, pivaloyl, t-butylacetyl, alanyl, phenylsulfonyl, benzyl, dimethoxybenzyl, [2-(trimethylsilyl)ethoxy]methyl (SEM), tetrahydropyranyl (THP), t-butyloxycarbonyl (Boc), and benzyloxycarbonyl (Cbz).

The term “sample” or “biological sample” as used herein, refers to a composition that is obtained or derived from a subject. The composition may include, but is not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, whole blood, blood-derived cells, cerebro-spinal fluid, saliva, sputum, perspiration, mucus, stool, tumors, tumor lysates, and tissue and tissue extracts such as homogenized tissue, tumor tissue, cellular extracts, and combinations thereof.

As used herein, a “stable control locus” refers to a region within a chromosome (e.g., a chromosomal band) of a cancer cell that is less prone to a duplication event, relative to another region (e.g., another chromosomal band) within the same chromosome of the cancer cell. A stable control locus is used as a control, or normalizer, for a target genomic locus being analyzed by a FISH assay.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer.

As used herein, a “target genomic locus” refers to any region within a chromosome (e.g., a locus, a chromosomal band, and/or one or more genes or genetic elements) in which amplification is being determined. Essentially, the target genomic locus is the experimental variable of a FISH assay.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, wax, nutrients, antibiotics, or the like. In some aspects, the sample is a tumor tissue sample. In some aspects, the tumor tissue sample is a UC tumor tissue sample (e.g., a bladder cancer tumor tissue sample (e.g., an MIBC tumor tissue sample)). In some aspects, the sample is a transurethral resection of bladder tumor (TURBT) sample. In some aspects, the sample is a cystectomy or nephroureterectomy sample. In other aspects, the tumor tissue sample is a lung cancer tumor tissue sample (e.g., an early stage lung cancer tissue sample (e.g., an NSCLC tumor tissue sample (e.g., a stage II, IIIA, or IIIB NSCLC tumor tissue sample), e.g., squamous or non-squamous NSCLC tumor tissue sample, e.g., a resectable NSCLC tumor tissue sample)). In some aspects, the sample is a locally advanced unresectable NSCLC tumor tissue sample (e.g., Stage IIIB NSCLC tumor tissue sample), or recurrent or metastatic NSCLC tumor tissue sample (e.g., Stage IV NSCLC tumor tissue sample), a pancreatic cancer tumor tissue sample (e.g., a PDAC tumor tissue sample), e.g., a metastatic PDAC tumor tissue sample), or a breast cancer tumor tissue sample (e.g., a HER2+ breast cancer tumor tissue sample or a TNBC tumor tissue sample).

“Treatment” and “treating,” as used herein, refer to the medical management of a subject with the intent to improve, ameliorate, stabilize, prevent or cure a disease or condition. This term includes active treatment (treatment directed to improve the disease or condition); causal treatment (treatment directed to the cause of the associated disease or condition); palliative treatment (treatment designed for the relief of symptoms of the disease or condition); preventative treatment (treatment directed to minimizing or partially or completely inhibiting the development of the associated disease or condition); and supportive treatment (treatment employed to supplement another therapy).

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

### BRIEF DESCRIPTION OF THE DRAWINGS

FIG. 1 is a bar plot illustrating that cyclin E1 (CCNE1) amplification is particularly frequent in uterine and ovarian cancer and shows the prevalence of CCNE1 amplification in tumor types from The Cancer Genome Atlas (TCGA). CCNE1 amplification frequency was calculated based on the Gistic2 values available in the TCGA PanCancer Atlas hosted at the publicly available cBioPortal website (www.cbioportal.org). Uterine Carcinosarcoma, Ovarian, and Uterine (Uterine Corpus Endometrial Carcinoma) are gynecological tumors.

FIG. 2 is a dot plot illustrating that the ploidy of chromosome (chr) bands varies between chromosomes and tumor types. Each data point in this plot corresponds to a chromosome band in the genome. The y-axis indicates the percentage of patients where the ploidy of a chromosomal band is equal to about 2 (e.g., 1.8-2), which was considered diploid. This information was calculated for different tumor indications such as TCGA-OV (ovarian), TCGA-UCEC (uterine, e.g., Uterine Corpus Endometrial Carcinoma), and TCGA-UCS (uterine carcinosarcoma). The x-axis separates the data for the chromosomes. This plot demonstrates that diploid frequency of bands varies within a chromosome, between chromosomes, and between indications. Furthermore, this plot demonstrates the challenge of identifying a chromosomal band that is frequently diploid across indications.

FIG. 3 is a dot plot illustrating that proximal bands within a chromosome show patterns of ploidy. Each data point in this plot corresponds to a chromosome band in the genome. The y-axis indicates the percentage of patients where the ploidy of a chromosomal band is equal to about 2 (e.g., 1.8-2) copies, which was considered diploid. This information was calculated for different tumor indications such as OV, UCEC, and UCS. The x-axis indicates the chromosome, chromosome arm, and central location of the band. This plot demonstrates that diploid frequency of bands varies along chromosomes in a non-random way. Deviation from diploid status occurs due to copy number change events, which can be large enough to affect multiple bands at once. This demonstrates that interrogation of the diploid percentage across the chromosome may identify groups of neighboring bands which have an increased diploid percentage.

FIG. 4 is a schematic of chromosome 19 (chr19) illustrating the relative location of a CCNE1 probe and exemplary control probes used for determining CCNE1 amplification. Chr19 undergoes significant rearrangement events in ovarian cancer. Cytogenetic abnormalities are particularly frequent on the p-arm of chr19, where control probes used in other assays are located. A subtelomeric q-arm band (e.g., chr19 q13.43) was used as stable control locus in the experiments described herein because this region is more frequently diploid than other regions on chr19 (e.g., FIGS. 5 and 6).

FIG. 5 is a dot plot showing that the band chr19 q13.43 was diploid at a high frequency in the indications shown. Each data point in this plot corresponds to a chromosome band in chr19 (chr19). The y-axis indicates the percentage of patients where the number of copies of a band is equal to about 2 (e.g., 1.8-2) copies, which was considered diploid. The x-axis shows tumor indications such as OV, UCEC, and UCS.

FIG. 6 is a dot plot showing that the band chr19 q13.43 is diploid at a high frequency in the indications shown. Each data point in this plot corresponds to a chromosome band in chr19. The y-axis indicates the percentage of patients where the number of copies of a band is equal to two, which is referred to as being diploid. This information is calculated for different tumor indications such as OV, UCEC, and UCS. The x-axis indicates the chromosome, chromosome arm, and central location of the band. Chromosomal band chr19 q13.43 did not show large deviations in diploid status, indicating that the chromosome region is more stable (i.e., less prone to a duplication event when compared to about 75% of the other bands present in the chromosome). This can be compared to the leftmost band in the TCGA-UCS portion of the plot which has a very high diploid status but is close to a band with much lower diploid status.

FIG. 7 shows a flow diagram of the final CCNE1 FISH assay pretreatment and hybridization conditions. The CCNE1 FISH assay was optimized for staining FFPE cell line pellets (left panel) and FFPE tumor tissue specimens (right panel). Pre-treatment conditions differ between these two types of samples, but protease and hybridization conditions are common. Representative images of cell pellet and tissue specimen staining are provided.

FIG. 8 shows representative images of ovarian and endometrial tumor tissue samples stained with the CCNE1 FISH assay. Shown here is a non-amplified endometrial tumor (upper left panel), an endometrial (upper right panel) and ovarian tumor (lower left panel) with low-level amplifications, and an ovarian tumor with high level amplification (lower right panel). Corresponding amplification status estimated by SNIPDx® and average (e.g., mean) of CCNE1/control signals per cell, estimated by FISH, are indicated below each image. Cyclin E protein H-score, estimated by immunohistochemistry (IHC) assay and pathologist review, is indicated for each specimen.

FIG. 9A is an overview of the CCNE1 FISH assay optimization and validation steps described herein (e.g., see Example 3 and 4). Assay conditions were optimized in a set of 6 FFPE tumor tissue samples and 6 cell lines, including amplified and non-amplified cases. Several iterations of protease pretreatments and hybridization temperature and conditions were tested to select the protocol with the best rate of signal vs noise ratio. Sensitivity and specificity of the assay were determined using samples described in FIG. 9B. Assay inter-day precision was performed using 5 tumor specimens (1 tissue with high copy number, 1 non-amplified tissue, and 3 specimens with amplification levels close to the threshold criteria described in FIG. 9B). Samples were assayed on 5 separate days. Intra-day precision was determined by assaying 5 tumor samples, selected with the same criteria as on the inter-day precision experiment, as five replicates on the same day.

FIG. 9B. is a dot plot of CCNE1 total copy number (TCN)-ploidy of formalin-fixed, paraffin-embedded (FFPE) tumor samples used in the CCNE1 FISH assay validation set. A total of 51 tissue specimens were used for validating sensitivity and specificity of the CCNE1 FISH assay, including 30 uterine cancer, three ovarian cancer, two gastro-intestinal tumors, five colorectal cancer and one triple negative breast cancer. A total of 10 normal tissue samples from ovary (n=5) and colon (n=5) were included as well. Tumor samples were profiled using the targeted next generation sequencing assay SNiPDx® as the baseline for amplification status CCNE1 copy number calculations. A total of 16 specimens in the uterine cancer cohort, three samples from the ovarian cancer cohort, and one sample from the gastro-intestinal cohort were considered amplified by SNiPDx®, using a threshold of

$$[ CCNE 1 Total Copy Number - Ploidy ] \geq 4$$

Conversely, 14 uterine tumor samples, one gastro-intestinal tumor, one triple negative breast cancer, and five colorectal cancer tissues were considered non-amplified by SNIPDx®.

FIG. 10 is a scatter plot of the CCNE1 FISH assay demonstrating >95% overall concordance with SNIPD®. From the 41 tumor samples (30 uterine cancer, 3 ovarian cancer, 2 gastro-intestinal tumors, 5 colorectal cancer and 1 triple negative breast cancer) included in the sensitivity/specificity test, 19 were determined as amplified by both assays and 20 reported as non-amplified, leading to an overall concordance of 95.1% (39/41 samples) between both assays. From the discordant samples, one specimen determined as amplified by SNIPDx® was reported as non-amplified for FISH and, conversely, one tumor sample deemed as non-amplified by SNIPDx® was reported as amplified by FISH (Sensitivity: 95.0% (19/20) and Specificity: 95.2% (20/21)). Amplification calls by SNiPDx® were determined using a threshold of

$$[ CCNE 1 Total Copy Number - Ploidy ] \geq 4$$

The threshold for reporting amplifications by FISH is a CCNE1-to-control probe ratio greater than or equal to two.

FIG. 11 is a dot plot of the inter-day reproducibility of the CCNE1 FISH assay. An inter-day precision assay was performed using 5 tumor specimens (1 tissue with high copy number, 1 non-amplified tissue and 3 specimens with amplification levels close to the threshold criteria described above). Samples were assayed on 5 separate days. The average (e.g., mean) coefficient of variation (CV) across five samples and 5 days was 14.78%.

FIG. 12 is a dot plot of the intra-day reproducibility of the CCNE1 FISH assay. An inter-day precision assay was performed using 5 tumor specimens (1 tissue with high copy number, 1 non-amplified tissue and 3 specimens with amplification levels close to the threshold criteria described in FIG. 11). Samples were assayed as 5 replicates on the same day. The average (e.g., mean) coefficient of variation (CV) across five samples was 4.47%.

FIG. 13 are plots showing that CCNE1 total copy number (TCN) calculations are highly consistent between FISH and SNIPDx® Next Generation Signaling (NGS) assays. Upper panel: the average (e.g., mean) CCNE1 signal per cell, estimated by FISH (Y axis), is significantly higher in tumor specimens deemed as CCNE1 amplified by SNIPDx® NGS assay (median 8.7, range 3.25-21.9), compared to non-amplified tumor tissue (median 2.75, range 1.85-5.95). In normal tissue, the average number of CCNE1 copies per cell is about 2, as expected for diploid and genetically stable specimens. Lower panel: correlation analysis between CCNE1 total copy number estimated by FISH (X axis) and SNIPDx® NGS assay (Y axis) shows a strong consistency between the two methodologies (Pearson p=0.923; m p-value=9.8 e-18).

### DETAILED DESCRIPTION

In general, the invention described herein provides methods of identifying a genomic locus that can serve as control (e.g., a stable control locus) for a fluorescence in situ hybridization (FISH) assay. FISH is a commonly used technique for determining amplification of a target genomic locus (e.g., a gene, such as CCNE1 (cyclin E1)) in a cell (e.g., a cancer cell). In general, the FISH assay utilizes two sets of FISH probes; the first set of probes (e.g., target probes) binds to the target genomic locus (e.g., a gene, e.g., CCNE1, or a portion thereof) and emits a first detectable signal, while the second set of probes (e.g., control probes) binds to a stable control locus (e.g., a CCNE1 control locus) and emits a second detectable signal. The ratio of the first detectable signal to the second detectable signal (e.g., the target genomic locus/stable control locus, e.g., CCNE1/control) can indicate amplification of the target genomic locus in the cell and, therefore, inform one about the likelihood of a cancerous cell. A stable control locus for one type of cancer, however, may not always work for another type of cancer; thus, there is a need for methods of identifying a stable control locus on a cancer-by-cancer basis. Doing so can allow for more accurate results of a FISH assay.

Advantageously, the methods described herein allow one to identify a proper stable control locus for any given cancer type or set of cancer types.

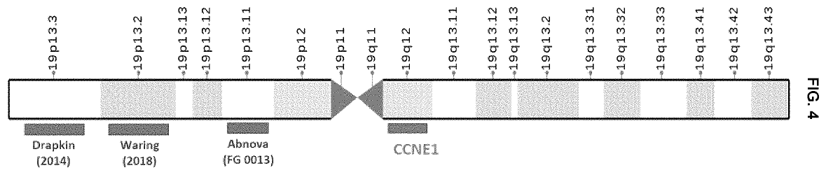

As a non-limiting example, methods of identifying a stable control locus (e.g., a CCNE1 control locus) for a FISH assay that detects CCNE1 amplification are described herein. The identified CCNE1 control loci were found to maintain a more constant ploidy in a cancer cell relative to other chromosomal loci used in the prior art (e.g., see FIG. 4). In other words, the CCNE1 control loci described herein were identified as having relatively few copy number variations (CNVs) in tumor cells (e.g., from ovarian (OV) and uterine cancer cells).

Thus, the methods described herein allow one to identify an expanded repertoire of stable control loci (e.g., a CCNE1 control locus) that may be utilized in the FISH methods described herein to accurately determine the amplification status of a target genomic locus (e.g., a gene, e.g., CCNE1) in a cell and, therefore, inform one about the likelihood of a cancerous cell.

### A. Methods of Identifying a Stable Control Locus

The present disclosure provides methods of identifying a stable control locus for use in a FISH assay. A stable control locus is a region within a chromosome (e.g., a chromosomal band) of a cancer cell that is less prone to a duplication event relative to another region (e.g., another chromosomal band) within the same chromosome. The stable control locus can be used as a control in a FISH assay because it is considered stable (i.e., the locus has a greater probability of maintaining its wildtype ploidy in a dividing cancer cell when compared to other loci on the same chromosome). For example, a stable control locus in a human cancer cell would be a locus that maintains a diploid state at least 2-fold, 3-fold, 4-fold, 5-fold, 6-fold, 7-fold, 8-fold, 9-fold, 10-fold, or greater, more often than that of a target genomic locus. In another example, a stable control locus in a human cancer cell would be a locus (e.g., a chromosomal band) that maintains a diploid state more frequently than at least 75% (e.g., at least 75%, 80%, 85%, 90%, or 95%) of the other loci (e.g., chromosomal bands) present on the same chromosome.

The method first includes the step of determining the ploidy of a plurality of chromosomal bands within a chromosome obtained from a biological sample of a subject. This first step should be repeated a plurality of times (e.g., 2, 3, 4, 5, 6, 7, 8, 9, 10, or more), wherein the biological sample (e.g., a tumor) is obtained from a different subject (e.g., a cancer subject) each time, thereby generating ploidy data from multiple samples for multiple subjects. Determining ploidy may be performed bioinformatically (e.g., as described in Example 1) by downloading and processing ploidy data from The Cancer Genome Atlas (TCGA), which can be found on the NIH Genomic Data Commons Data Portal (portal.gdc.cancer.gov/). However, if ploidy data are not available for a desired sample type (e.g., a sample having a desired disease or condition), one can utilize Next Generation Sequencing (NGS) with their sample of interest to generate it.

Next, the method includes the steps of determining a weighted average (e.g., mean) ploidy for each chromosomal band in the chromosome, and then determining the percentage of subjects that are diploid at each chromosomal band. A diploid subject for a given chromosomal band is one with a weighted average ploidy between 1.8 copies and 2.2 copies (e.g., 1.8 copies, 1.9 copies, 2 copies, 2.1 copies, or 2.2 copies) of the chromosomal band.

Lastly, the method includes the steps of ranking, from high to low, each chromosomal band by the percentage of subjects that were considered diploid. Any chromosomal band identified in the upper quartile (e.g., 75^{th}, 80^{th}, 85^{th}, 90^{th}, 95^{th}, 96^{th}, 97^{th}, 98^{th}, 99^{th} or 100^{th} percentile) of this rank may be considered and selected to be a stable control locus for use in the FISH assays described in Section B.

Ideally, the identified stable control locus should only be used in a FISH assay that is analyzing a target genomic locus from a biological sample having a similar disease or condition. For example, if a stable control locus is identified using OV tumor samples, then that stable control locus should only be used in a FISH assay analyzing a target genomic locus in an OV tumor sample. In another example, if a stable control locus is identified using OV and uterine corpus endometrial carcinoma (UCEC) tumor samples, then that stable control locus should only be used in a FISH assay analyzing a target genomic locus in an OV and/or UCEC tumor sample. In yet another example, if a stable control locus is identified using OV, UCEC, and uterine carcinosarcoma (UCS) tumor samples, then that stable control locus should only be used in a FISH assay analyzing a target genomic locus in an OV, UCEC, and/or USC tumor sample.

### B. Methods of Determining Amplification of a Target Genomic Locus

The present disclosure provides methods of determining if a target genomic locus (e.g., a gene, e.g., CCNE1) within one or more chromosomal DNA molecules in a population of cells is amplified or not amplified. Determining such amplification can inform one of the likelihood of the presence of a cancerous cell.

The method first includes the step of contacting (e.g., in vitro) the population of cells (e.g., a tumor) having chromosomal DNA (e.g., nuclear and/or mitochondrial DNA) with a plurality of target probes and a plurality of control probes. The target probes can emit a first detectable signal and are complementary to the target genomic locus (e.g., a gene, e.g., CCNE1) while the control probes can emit a second detectable signal and are complementary to a stable control locus (e.g., a CCNE1 control locus). In a first example, the plurality of target probes may be complementary to the q12 band of chromosome (chr) 19 (chr19 q12), relative to Homo sapiens genome assembly NCBI36. In a second example, the plurality of target probes may be complementary to the CCNE1 gene, or a portion thereof. In either the first or second example, the control probe may be complementary to a stable control locus (e.g., a CCNE1 control locus) selected from the following: chr19 q13.43, chr19 q13.41, chr19 q13.42, chr19 p13.3, chr19 q13.32, chr19 q13.33, chr19 q13.31, chr19 p13.13, or chr19 q13.13 (relative to Homo sapiens genome assembly NCBI36).

Next, the method includes the step of quantifying the total number of first detectable signals in a cell, quantifying the total number of second detectable signals in the same cell, and then dividing the total number of first detectable signals by the total number of second detectable signals, thereby determining a signal ratio (e.g., CCNE1/control) for that cell. This step can be repeated a plurality of times, wherein the cell is different each time, thereby determining multiple signal ratios within the cell population (e.g., tumor). For example, this step may be repeated 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. Preferably, this step is repeated at least 50 times, thereby determining at least 50 (e.g., 50-60, 50-70, 50-80, 50-90, 50-100) signal ratios within the cell population.

Lastly, the method includes the step of determining an average (e.g., mean) signal ratio for the cell population. If the average signal ratio is ≥2 (e.g., ≥2, ≥2.5, ≥3, ≥3.5, ≥4, ≥4.5, ≥5, ≥6, ≥7, ≥8, ≥9, ≥10), then the target genomic locus (e.g., a gene, e.g., CCNE1) is considered amplified. If the average signal ratio is <2 (e.g., <1.9, <1.8, <1.7, <1.6, <1.5, <1, <0.5), then the target genomic locus (e.g., a gene, e.g., CCNE1) is not considered amplified. If a target genomic locus (e.g., a gene, e.g., CCNE1) is considered amplified, then the subject may be treated with an effective amount of a Myt1 inhibitor, as detailed further in Section B below.

Further details on the target genomic locus, the stable control locus (e.g., the CCNE1 control locus), the target probe, the control probe, and the type of cells or samples to be analyzed are described below.

i. Target Genomic Locus

The target genomic locus may be any stretch of nucleic acids within one or more chromosomal DNA (e.g., nuclear and/or mitochondrial) molecules. Typically, the target genomic locus resides within one chromosome and contains at least one gene, genetic element, mobile genetic element, exon, and/or intron. For example, the target genomic locus may be the CCNE1 gene, a region containing the CCNE1 gene, or a region containing a portion of the CCNE1 gene. The target genomic locus is generally present within a tissue sample (e.g., a tumor), a cell (e.g., a cancer cell), or any population of cells derived from a sample (e.g., a tissue).

The target genomic locus (e.g., a gene, e.g., CCNE1) may be a stretch of nucleotides that is about 5,000 to about 500,000 nucleotides in length (e.g., about 5,000 to about 200,000 nucleotides in length, about 150,000 to about 300,000 nucleotides in length, or about 250,000 to about 500,000 nucleotides in length), about 5,000 to about 300,000 nucleotides in length (e.g., about 5,000 to about 150,000 nucleotides in length, about 100,000 to about 200,000 nucleotides in length, or about 150,000 to about 300,000 nucleotides in length), about 50,000 to about 300,000 nucleotides in length (e.g., about 50,000 to about 150,000 nucleotides in length, about 100,000 to about 250,000 nucleotides in length, or about 200,000 to about 300,000 nucleotides in length), about 100,000 to about 300,000 nucleotides in length (e.g., about 150,000 to about 275,000 nucleotides in length, about 150,000 to about 250,000 nucleotides in length, or about 150,000 to about 225,000 nucleotides in length), about 150,000 to about 300,000 nucleotides in length (e.g., about 150,000 to about 240,000 nucleotides in length, about 160,000 to about 230,000 nucleotides in length, or about 170,000 to about 220,000 nucleotides in length), about 150,000 to about 250,000 nucleotides in length (e.g., about 160,000 to about 240,000 nucleotides in length, about 170,000 to about 230,000 nucleotides in length, or about 180,000 to about 220,000 nucleotides in length), or about 175,000 to about 225,000 nucleotides in length (e.g., about 180,000 to about 215,000 nucleotides in length, about 190,000 to about 210,000 nucleotides in length, or about 195,000 to about 205,000 nucleotides in length).

For example, the target genomic locus (e.g., a gene, e.g., CCNE1) may be about 5,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000, 155,000, 160,000, 165,000, 170,000, 175,000, 180,000, 185,000, 190,000, 195,000, 196,196, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 405,000, 410,000, 415,000, 420,000, 425,000, 430,000, 435,000, 440,000, 445,000, 450,000, 455,000, 460,000, 465,000, 470,000, 475,000, 480,000, 485,000, 490,000, 495,000, 500,000 nucleotides in length.

ii. Stable Control Locus

A stable control locus is a region within a chromosome (e.g., a chromosomal band) of a cancer cell that is less prone to a duplication event relative to another region (e.g., another chromosomal band) within the same chromosome. The stable control locus can be used as a control for a target genomic locus because it is considered more stable; in other words, the stable control locus (e.g., a CCNE1 control locus) has a greater probability of maintaining its wildtype ploidy (e.g., a diploid locus for humans) in a dividing cancer cell. For example, a stable control locus in a human cell would be a locus that maintains a diploid state at least 2-fold, 3-fold, 4-fold, 5-fold, 6-fold, 7-fold, 8-fold, 9-fold, 10-fold, or greater, more often than that of a target genomic locus. In another example, a stable control locus in a human cell would be a locus (e.g., a chromosomal band) that maintains a diploid state more frequently than at least 75% (e.g., at least 75%, 80%, 85%, 90%, or 95%) of the other loci (e.g., chromosomal bands) present on the same chromosome. Methods of identifying a stable control locus are described in Section A above. Experiments describing the identification of several CCNE1 control loci are described in Example 1 below.

Ideally, the stable control locus should only be used in a FISH assay that is analyzing a target genomic locus from a biological sample having a similar cancer. In other words, if a stable control locus was identified using OV tumor samples, then the stable control locus should only be used in a FISH assay analyzing a target genomic locus in an OV tumor sample. In another example, if a stable control locus was identified using OV and UCEC tumor samples, then the stable control locus should only be used in a FISH assay analyzing a target genomic locus in an OV and/or UCEC tumor sample. In yet another example, if a stable control locus was identified using OV, UCEC, and UCS tumor samples, then the stable control locus should only be used in a FISH assay analyzing a target genomic locus in an OV, UCEC, and/or USC tumor sample.

The stable control locus (e.g., a CCNE1 control locus) can be a stretch of nucleic acids residing in a human chromosome (e.g., nuclear and/or mitochondrial), such as a chromosomal band or a region within a chromosomal band. The stable control locus resides on the same chromosome as the target genomic locus. For example, when the target genomic locus contains the CCNE1 gene (which is on chr 19), the stable control locus may be a CCNE1 control locus selected from the following: chr19 q13.43, chr19 q13.41, chr19 q13.42, chr19 p13.3, chr19 q13.32, chr19 q13.33, chr19 q13.31, chr19 p13.13, or chr19 q13.13 (relative to Homo sapiens genome assembly NCBI36). For example, the stable control locus may reside within nucleotides 29,644,930-29,841,126 of chr 19 q13.43, relative to the Homo sapiens genome assembly NCBI36.

The stable control locus (e.g., a CCNE1 control locus) may be a stretch of nucleotides that is about 5,000 to about 500,000 nucleotides in length (e.g., about 5,000 to about 200,000 nucleotides in length, about 150,000 to about 300,000 nucleotides in length, or about 250,000 to about 500,000 nucleotides in length), about 5,000 to about 300,000 nucleotides in length (e.g., about 5,000 to about 150,000 nucleotides in length, about 100,000 to about 200,000 nucleotides in length, or about 150,000 to about 300,000 nucleotides in length), about 50,000 to about 300,000 nucleotides in length (e.g., about 50,000 to about 150,000 nucleotides in length, about 100,000 to about 250,000 nucleotides in length, or about 200,000 to about 300,000 nucleotides in length), about 100,000 to about 300,000 nucleotides in length (e.g., about 150,000 to about 275,000 nucleotides in length, about 150,000 to about 250,000 nucleotides in length, or about 150,000 to about 225,000 nucleotides in length), about 150,000 to about 300,000 nucleotides in length (e.g., about 150,000 to about 240,000 nucleotides in length, about 160,000 to about 230,000 nucleotides in length, or about 170,000 to about 220,000 nucleotides in length), about 150,000 to about 250,000 nucleotides in length (e.g., about 160,000 to about 240,000 nucleotides in length, about 170,000 to about 230,000 nucleotides in length, or about 180,000 to about 220,000 nucleotides in length), or about 175,000 to about 225,000 nucleotides in length (e.g., about 180,000 to about 215,000 nucleotides in length, about 190,000 to about 210,000 nucleotides in length, or about 195,000 to about 205,000 nucleotides in length).

For example, the stable control locus (e.g., a CCNE1 control locus) may be about 5,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000, 155,000, 160,000, 165,000, 170,000, 175,000, 180,000, 185,000, 190,000, 195,000, 196,196, 200,000, 205,000, 210,000, 215,000, 220,000, 225,000, 230,000, 235,000, 240,000, 245,000, 250,000, 255,000, 260,000, 265,000, 270,000, 275,000, 280,000, 285,000, 290,000, 295,000, 300,000, 305,000, 310,000, 315,000, 320,000, 325,000, 330,000, 335,000, 340,000, 345,000, 350,000, 355,000, 360,000, 365,000, 370,000, 375,000, 380,000, 385,000, 390,000, 395,000, 400,000, 405,000, 410,000, 415,000, 420,000, 425,000, 430,000, 435,000, 440,000, 445,000, 450,000, 455,000, 460,000, 465,000, 470,000, 475,000, 480,000, 485,000, 490,000, 495,000, 500,000 nucleotides in length.

iii. Samples

The target genomic locus (e.g., a gene, e.g., CCNE1) and the stable control locus (e.g., a CCNE1 control locus) may be present within a sample, such as a biological sample from a subject (e.g., a human). Non-limiting examples of biological samples include blood, serum, plasma, saliva, a cell, a cell culture, a tissue (e.g., skin, liver, kidneys, heart, lung, muscle), and a tumor (e.g., a benign or cancerous tumor). The subject could be any mammal, such as, but not limited to, a human, monkey, cat, dog, mouse, pig, cow, horse, sheep, and bat.

iv. The Target Probe

The target probe is a nucleic acid molecule capable of hybridizing to the target genomic locus (e.g., a gene, e.g., CCNE1) and emitting a first detectable signal (e.g., a fluorescent signal).

The target probe may be an allele-specific probe that discriminates between single nucleotide variations (SNV) within the target genomic locus.

The target probe may be a nucleic acid molecule that is about 5,000 to about 40,000 nucleotides in length (e.g., about 5,000 to about 10,000 nucleotides in length, about 7,000 to about 15,000 nucleotides in length, about 5,000 to about 20,000 nucleotides in length, about 10,000 to about 20,000 nucleotides in length, about 15,000 to about 30,000 nucleotides in length, about 20,000 to about 35,000 nucleotides in length, or about 25,000 to about 40,000 nucleotides in length). For example, the target probe may be about 5,000, about 6,000, about 7,000, about 8,000, about 9,000, about 10,000, about 11,000, about 12,000, about 13,000, about 14,000, about 15,000, about 16,000, about 17,000, about 18,000, about 19,000, about 20,000, about 21,000, about 22,000, about 23,000, about 24,000, about 25,000, about 26,000, about 27,000, about 28,000, about 29,000, about 30,000, about 31,000, about 32,000, about 33,000, about 34,000, about 35,000, about 36,000, about 37,000, about 38,000, about 39,000, about 40,000 nucleotides in length.

v. The Control Probe

The control probe may be a nucleic acid molecule capable of hybridizing to a stable control locus (e.g., a CCNE1 control locus) and emitting a first detectable signal (e.g., a fluorescent signal).

The control probe may be capable of hybridizing within the chr19 q13.43 chromosomal region (e.g., nucleotides 29,644,930-29,841,126 of chr 19), relative to the Homo sapiens genome assembly NCBI36.

The control probe may be a nucleic acid molecule that is about 5,000 to about 40,000 nucleotides in length (e.g., about 5,000 to about 10,000 nucleotides in length, about 7,000 to about 15,000 nucleotides in length, about 5,000 to about 20,000 nucleotides in length, about 10,000 to about 20,000 nucleotides in length, about 15,000 to about 30,000 nucleotides in length, about 20,000 to about 35,000 nucleotides in length, or about 25,000 to about 40,000 nucleotides in length). For example, the control probe may be about 5,000, about 6,000, about 7,000, about 8,000, about 9,000, about 10,000, about 11,000, about 12,000, about 13,000, about 14,000, about 15,000, about 16,000, about 17,000, about 18,000, about 19,000, about 20,000, about 21,000, about 22,000, about 23,000, about 24,000, about 25,000, about 26,000, about 27,000, about 28,000, about 29,000, about 30,000, about 31,000, about 32,000, about 33,000, about 34,000, about 35,000, about 36,000, about 37,000, about 38,000, about 39,000, about 40,000 nucleotides in length.

vi. Modified Probes

It is contemplated that the target and/or control probes described herein may be used in a modified form. Typically, modifications to nucleic acid molecules are introduced to optimize the molecule's efficacy or biophysical properties (e.g., increasing stability and/or targeting to a particular location or cell type). Such modifications may be achieved by generating chimeric nucleic acid molecules that contain any combination of deoxyribonucleic acids (DNA), ribonucleic acids (RNA), locked nucleic acids (LNA), bridged nucleic acids (BNA), and/or a peptide nucleic acids (PNA). Furthermore, modifications may include the incorporation of, for example, one or more alternative nucleosides, alternative 2′ sugar moieties, and/or alternative internucleoside linkages described herein.

### Nucleoside Modifications

Modification of the target and/or control probes described in the methods herein include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The oligonucleotides may also include nucleobases in which the purine or pyrimidine base is replaced with other heterocycles, for example 7-deaza-adenine, 7-deazaguanosine, 2-aminopyridine, and/or 2-pyridone. Further modification of the oligonucleotides may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

### Sugar Modifications

Modifications of the target and/or control probes described in the methods herein may also include one or more of the following 2′ sugar modifications: 2′-O-methyl (2′-O-Me), 2′-methoxyethoxy (2′-O—CH2CH2OCH3, also known as 2′-O-(2-methoxyethyl) or 2′-MOE), 2′-dimethylaminooxyethoxy, i.e., a O(CH2)2ON(CH3)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH2OCH2N(CH3)₂. Other possible 2′-modifications that can modify the oligonucleotides include all possible orientations of OH; F; O—, S—, or N-alkyl; O—, S—, or N-alkenyl; O-, S- or N-alkynyl; or O-alkyl-O-alkyl, wherein the alkyl, alkenyl and alkynyl may be substituted or unsubstituted C1 to C10 alkyl or C₂ to C10 alkenyl and alkynyl. Other potential sugar substituent groups include, e.g., aminopropoxy (—OCH2CH2CH2NH₂), allyl (—CH2-CH═CH2), —O-allyl (—O—CH2-CH═CH2) and fluoro (F). 2′-sugar substituent groups may be in the arabino (up) position or ribo (down) position. In some embodiments, the 2′-arabino modification is 2′-F. Similar modifications may also be made at other positions on the interfering RNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Internucleoside Linkage Modifications

Modifications of the target and/or control probes described in the methods herein may include one or more of the following internucleoside modifications: phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

### C. Methods of Treatment

The present disclosure provides methods of identifying and/or treating a patient at risk of having cancer or suspected of having cancer. The method includes identifying a subject as having cancer or suspected of having a cancer by performing any of the methods described in Section A above. For example, a sample of cells or tissue (e.g., a tumor or a population of cells derived from a tumor) from a subject can be obtained and the amplification status of a target genomic locus (e.g., a gene, e.g., CCNE1) within the sample can be determined via the methods described in Section A. The target genomic locus may be CCNE1. For example, amplification of CCNE1 in a biological sample (e.g., a tumor or a population of cells derived from a tumor) indicates an increased likelihood of a subject having a cancer or an increased risk of developing a cancer, relative to a subject without a CCNE1 amplification. Such a subject may be treated using the methods described herein. The method of treating a subject at risk of having cancer or suspected of having cancer includes administering an effective amount of an inhibitor of a membrane-associated tyrosine and threonine-specific cdc2-inhibitory kinase (Myt1).

i. Myt1 Inhibitors

Myt1 inhibitors that may be used in the methods of the invention include those disclosed, e.g., in international patent application publication Nos. WO 2021/195781 and WO 2023/220831, and in U.S. patent application Ser. Nos. 17/937,977; 17/961,103; and Ser. No. 18/140,302, the disclosures of which are incorporated herein in their entirety.

The Myt inhibitor may be, e.g., a compound of formula (I):

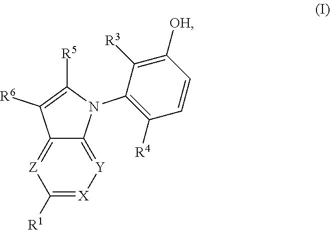

or a pharmaceutically acceptable salt thereof,
where
   each of X, Y, and Z is independently N or CR²;
   R¹ and each R² are independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁷)₂, —OR⁷, —C(O)N(R⁸)₂, —SO₂N(R⁸)₂, —SO₂R^{7A}, or -Q-R^{7B}; or R¹ combines with one R² that is vicinal to R¹ to form an optionally substituted C₃₋₆ alkylene;
   each of R³ and R⁴ is independently optionally substituted C₁₋₆ alkyl or halogen;
   R⁵ is H or —N(R⁷)₂;
   R⁶ is —C(O)NH(R⁸), —C(O)R^{7A}, or —SO₂R^{7A};
   each R⁷ is independently hydrogen, optionally substituted C₁₋₆ alkyl, optionally substituted C₆₋₁₀ aryl C₁₋₆ alkyl, optionally substituted C₆₋₁₀ aryl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;
   each R^{7A} is independently optionally substituted C₁₋₆ alkyl, optionally substituted C₃₋₈ cycloalkyl, or optionally substituted C₆₋₁₀ aryl;
   each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;
   each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;
   Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene.

Preferably, the compound of formula (I) is enriched for the atropisomer of formula (IA):

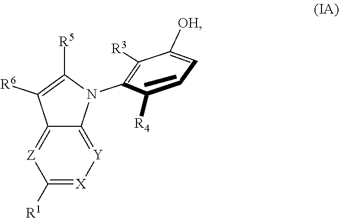

where all variables are as described herein.

The compound of the invention may be, e.g., a compound of formula (II):

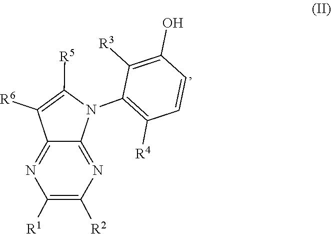

where all variables are as described herein.

Preferably, the compound of formula (II) is enriched for the atropisomer of formula (IIA):

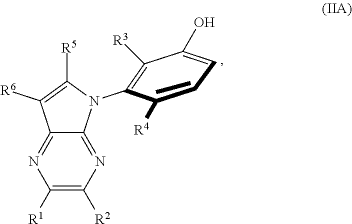

where all variables are as described herein.

The compound of the invention may be, e.g., a compound of formula (III):

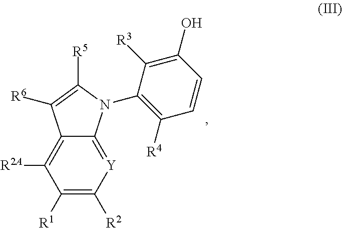

where R^{2A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁷)₂, —OR⁷, —C(O)N(R⁸)₂, —SO₂N(R⁸)₂, —SO₂R^{7A}, or -Q-R^{7B}.

Preferably, the compound of formula (III) is enriched for the atropisomer of formula (IIIA):

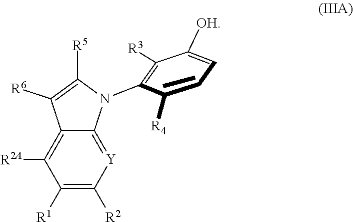

The Myt1 inhibitor may be, e.g., a compound of formula (IV):

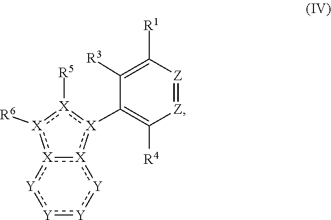

or a pharmaceutically acceptable salt thereof,
where
   each [symbol] is a single or double bond;
   one, two, or three X groups are N, and the remaining X groups are C;
   each Y is independently N or CR²;
   each Z is independently N or CH;
   R¹ is OH, and R³ is hydrogen, optionally substituted C₁₋₆ alkyl, halogen, or optionally substituted C₃₋₈ cycloalkyl; or R¹ and R³ combine to form —CR⁹═N—NH—;
   each R² is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₈₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B};
   R⁴ is independently hydrogen, optionally substituted C₁₋₆ alkyl, or halogen;
   R⁵ is hydrogen, halogen, or —N(R⁷)₂;
   R⁸ is —C(O)NH(R⁸), —C(O)R^{7A}, or —SO₂R^{7A};
   each R⁷ is independently hydrogen, optionally substituted C₁₋₈ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;
   each R^{7A} is independently optionally substituted C₁₋₆ alkyl, optionally substituted C₃₋₈ cycloalkyl, or optionally substituted C₆₋₁₀ aryl;
   each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;
   each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;
   R⁹ is hydrogen or halogen; and
   Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene.

In some embodiments, one X group is N, and the remaining X groups are C. In some embodiments, all Y groups are CR². In some embodiments, one Y group is N, and the remaining Y groups are CR². In some embodiments, two Y group are N, and the remaining Y groups are CR².

In some embodiments, R¹ is OH, and R³ is hydrogen, optionally substituted C₁₋₆ alkyl, or halogen. In some embodiments, R¹ is OH, and R³ is optionally substituted C₁₋₆ alkyl or halogen. In some embodiments, R¹ and R³ combine to form —CR⁹═N—NH— (e.g., R⁹ is hydrogen).

In some embodiments, the Myt1 inhibitor is of formula (V):

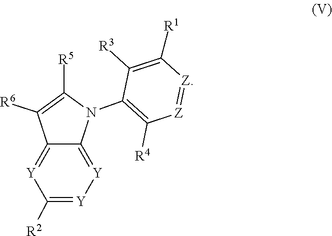

In some embodiments, the Myt1 inhibitor is:

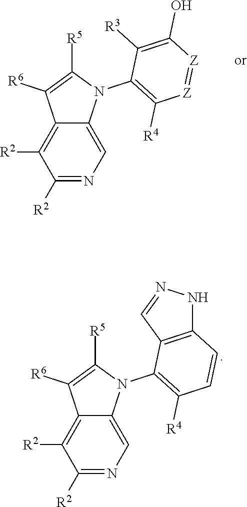

In some embodiments, the Myt1 inhibitor is enriched for the atropisomer of formula (V-i):

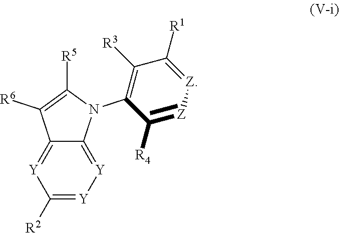

In some embodiments, the Myt1 inhibitor is enriched for the following atropisomer:

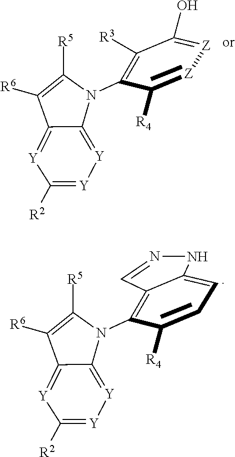

In some embodiments, the Myt1 inhibitor is of formula (VA):

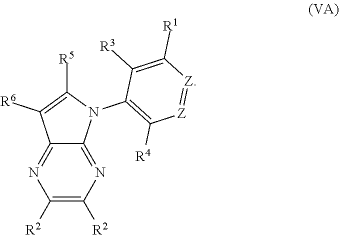

In some embodiments, the Myt1 inhibitor is:

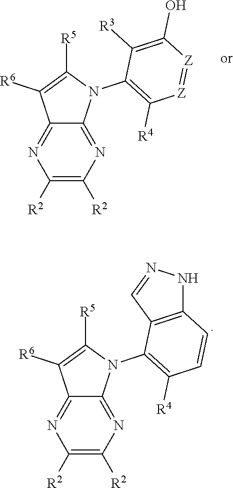

In some embodiments, the Myt1 inhibitor is enriched for the atropisomer of (VA-i):

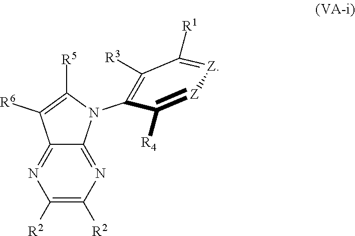

In some embodiments, the Myt1 inhibitor is enriched for the following atropisomer:

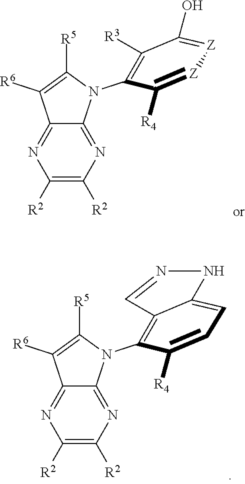

In some embodiments, the Myt1 inhibitor is of formula (VB):

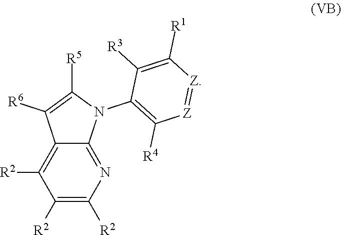

In some embodiments, the Myt1 inhibitor is:

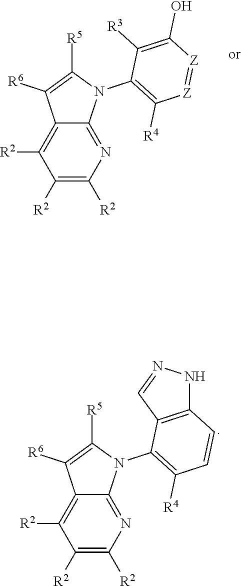

In some embodiments, the Myt1 inhibitor is enriched for the atropisomer of (VB-i):

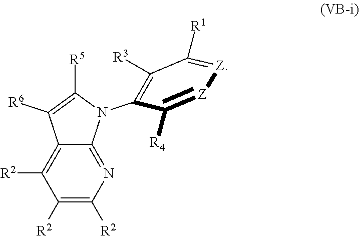

In some embodiments, the compound is enriched for the following atropisomer:

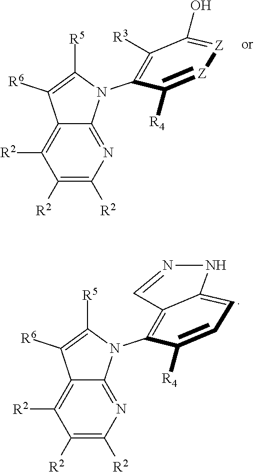

In some embodiments, the Myt1 inhibitor is of formula (VC):

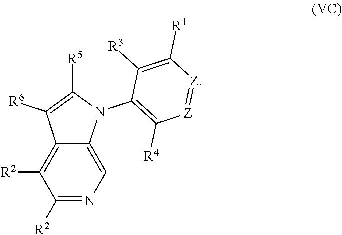

In some embodiments, the Myt1 inhibitor is:

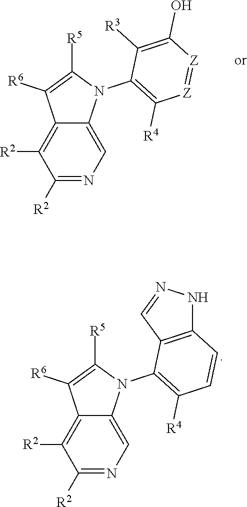

In some embodiments, the Myt1 inhibitor is enriched for the atropisomer of (VC-i):

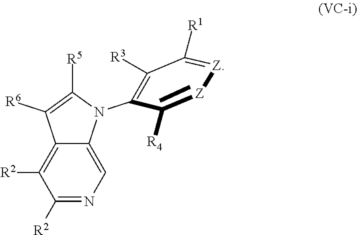

In some embodiments, the Myt1 inhibitor is enriched for the following atropisomer:

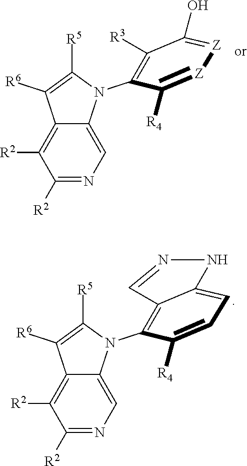

In some embodiments, the Myt1 inhibitor is of formula (VD):

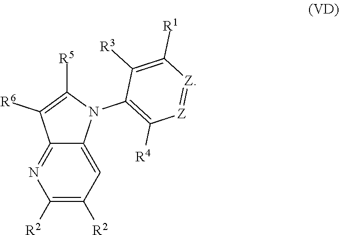

In some embodiments, the Myt1 inhibitor is:

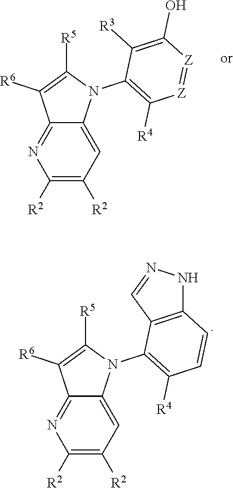

In some embodiments, the Myt1 inhibitor is enriched for the atropisomer of (VD-i):

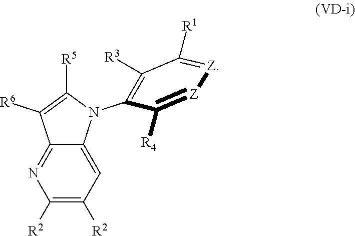

In some embodiments, the Myt1 inhibitor is enriched for the following atropisomer:

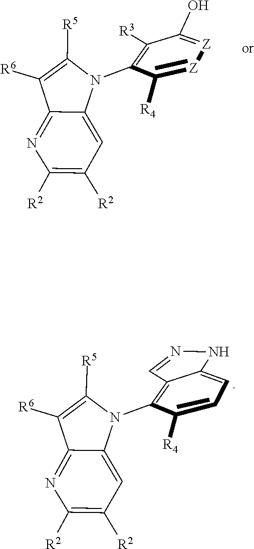

In some embodiments, the Myt1 inhibitor is of formula (VI):

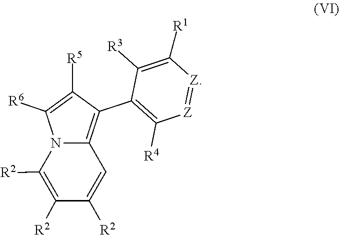

In some embodiments, the Myt1 inhibitor is:

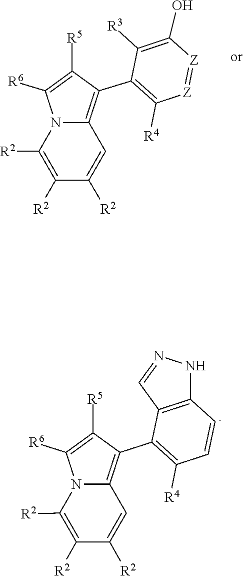

In some embodiments, the Myt1 inhibitor is enriched for the atropisomer of (VI-i):

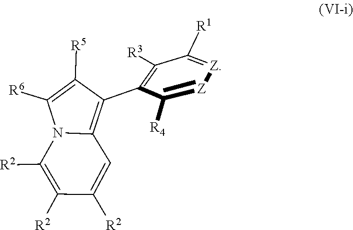

In some embodiments, the Myt1 inhibitor is enriched for the following atropisomer:

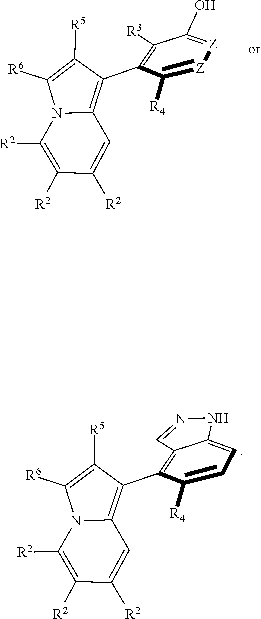

In some embodiments, the Myt1 inhibitor is of formula (VII):

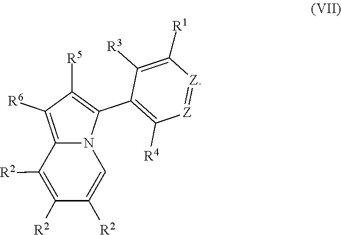

In some embodiments, the Myt1 inhibitor is:

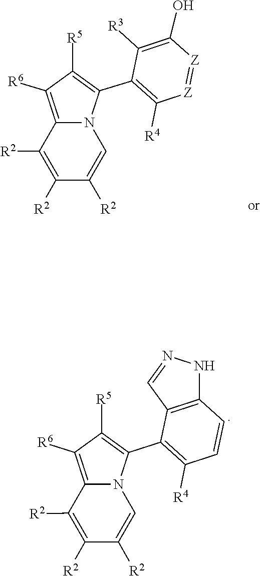

In some embodiments, the compound is enriched for the atropisomer of (VII-i):

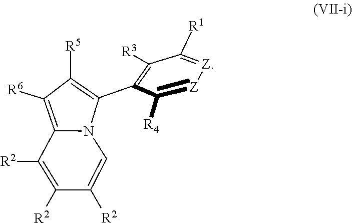

In some embodiments, the Myt1 inhibitor is enriched for the following atropisomer:

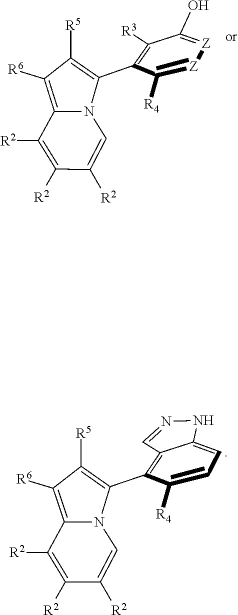

In some embodiments, the Myt1 inhibitor is of formula (VIII):

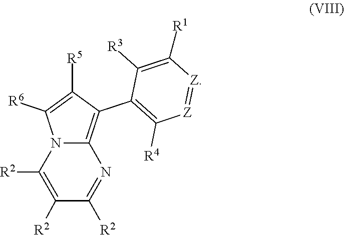

In some embodiments, the Myt1 inhibitor is:

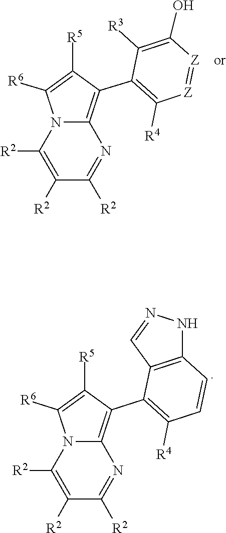

In some embodiments, the Myt1 inhibitor is enriched for the atropisomer of (VIII-i):

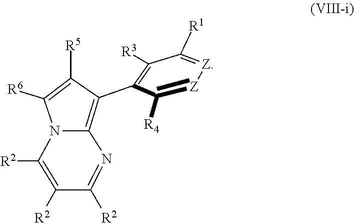

In some embodiments, the Myt1 inhibitor is enriched for the following atropisomer:

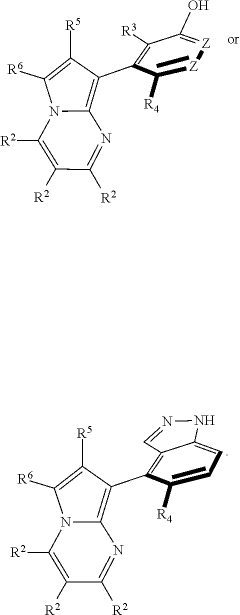

In some embodiments, the Myt1 inhibitor is of formula (IX)

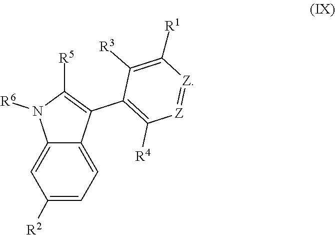

The Myt1 inhibitor may be, e.g., a compound of formula (XA):

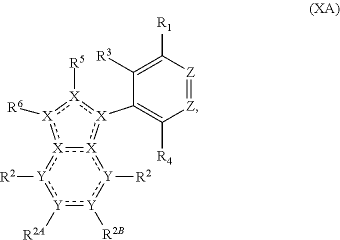

or a pharmaceutically acceptable salt thereof,
wherein
   each [symbol] is a single or double bond;
   one, two, or three X groups are N, and the remaining X groups are C;
   each Y is independently N or C;
   each Z is independently N or CH;
   R¹ is OH, and R³ is hydrogen, optionally substituted C₁₋₆ alkyl, halogen, or optionally substituted C₃₋₈ cycloalkyl; or R¹ and R³ combine to form —CR⁹═N—NH—;
   each R² is independently absent, hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B}; and R^{2A} and R^{2B}, together with the atoms to which they are attached, combine to form ring A; or R² and R^{2A}, together with the atoms to which they are attached, combine to form ring A, and R^{2B} is absent or hydrogen;
   R⁴ is hydrogen, optionally substituted C₁₋₆ alkyl, halogen, or optionally substituted C₃₋₈ cycloalkyl;
   R⁵ is hydrogen, halogen, or —N(R⁷)₂;
   R⁶ is —C(O)NH(R⁸), —C(O)R^{7A}, or —SO₂R^{7A};
   each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;
   each R^{7A} is independently optionally substituted C₁₋₆ alkyl, optionally substituted C₃₋₈ cycloalkyl, or optionally substituted C₆₋₁₀ aryl;
   each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;
   each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;
   R⁹ is hydrogen or halogen;
   ring A is a 5- or 6-membered carbocyclic ring or a 5- or 6-membered heterocyclic ring, wherein A is optionally substituted with 1, 2, 3, or 4 non-hydrogen R² groups; and
   Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene;
   wherein each R² is absent, if attached to Y that is N.

The Myt1 inhibitor may be, e.g., a compound of formula (X):

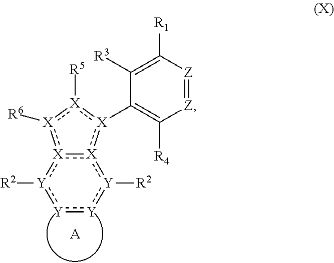

or a pharmaceutically acceptable salt thereof,
where
   each [symbol] is a single or double bond;
   A is a 5- or 6-membered carbocyclic ring or a 5- or 6-membered heterocyclic ring, where A is optionally substituted with 1, 2, 3, or 4 non-hydrogen R² groups;
   one, two, or three X groups are N, and the remaining X groups are C;
   each Y is independently N or C;
   each Z is independently N or CH;
   R¹ is OH, and R³ is hydrogen, optionally substituted C₁₋₆ alkyl, halogen, or optionally substituted C₃₋₈ cycloalkyl; or R¹ and R³ combine to form —CR⁹═N—NH—;
   each R² is independently absent, hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B};
   R⁴ is hydrogen, optionally substituted C₁₋₆ alkyl, halogen, or optionally substituted C₃₋₈ cycloalkyl;
   R⁵ is hydrogen, halogen, or —N(R⁷)₂;
   R⁶ is —C(O)NH(R³), —C(O)R^{7A}, or —SO₂R^{7A};
   each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;
   each R^{7A} is independently optionally substituted C₁₋₆ alkyl, optionally substituted C₃₋₈ cycloalkyl, or optionally substituted C₆₋₁₀ aryl;
   each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;
   each R⁸ is independently hydrogen, optionally substituted C₁₋₆ alkyl, optionally substituted C₂₋₈ alkoxyalkyl, optionally substituted C₈₋₁₀ aryl C₁₋₈ alkyl, optionally substituted C₈₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;
   R⁹ is hydrogen or halogen; and
   Q is optionally substituted C₁₋₈ alkylene, optionally substituted C₂₋₈ alkenylene, optionally substituted C₂₋₈ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene;
   where each R² is absent, if attached to Y that is N.

In some embodiments, one X group is N, and the remaining X groups are C. In some embodiments, all Y groups are C. In some embodiments, one Y group is N, and the remaining Y groups are C. In some embodiments, two Y group are N, and the remaining Y groups are C.

In some embodiments, the Myt1 inhibitor is of formula (XI):

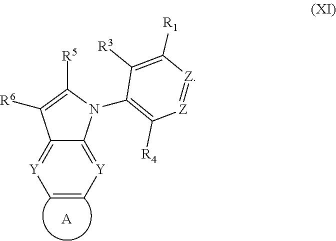

In some embodiments, the Myt1 inhibitor is enriched for the atropisomer of formula (XI-i):

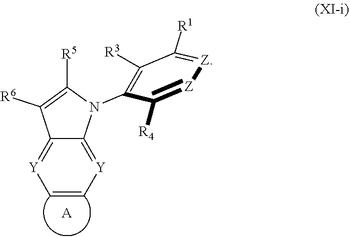

In some embodiments, the Myt1 inhibitor is of formula (XIA):

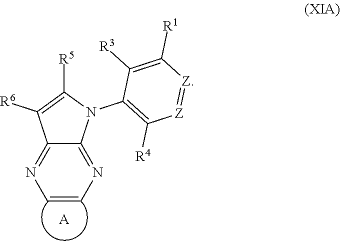

In some embodiments, the Myt1 inhibitor is enriched for the atropisomer of formula (XIA-i):

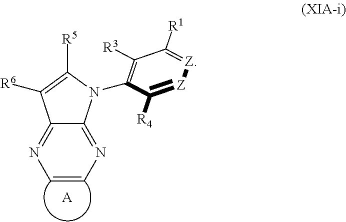

In some embodiments, the Myt1 inhibitor is of formula (XIA-ii):

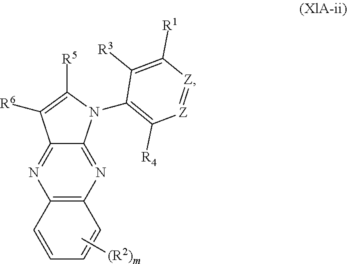

where m is 0, 1, 2, 3, or 4.

In some embodiments, the Myt1 inhibitor is enriched for the atropisomer of formula (XIA-iii):

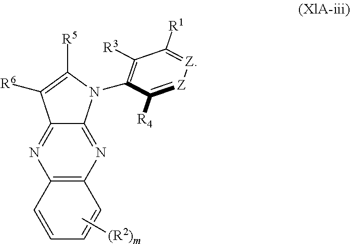

In some embodiments, the Myt1 inhibitor is of formula (XIA-iv):

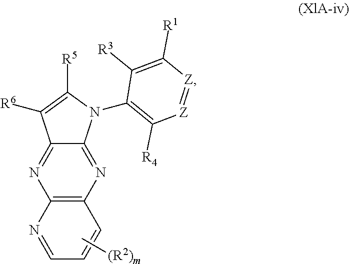

where m is 0, 1, 2, or 3.

In some embodiments, the Myt1 inhibitor is enriched for the atropisomer of formula (XIA-v):

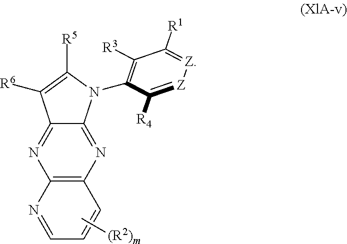

In some embodiments, the Myt1 inhibitor is of formula (XIA-vi):

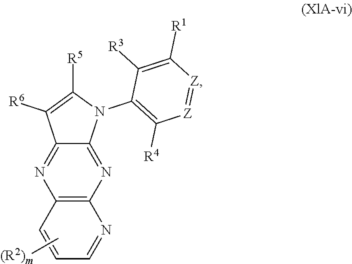

where m is 0, 1, 2, or 3.

In some embodiments, the Myt1 inhibitor is enriched for the atropisomer of formula (XIA-vii):

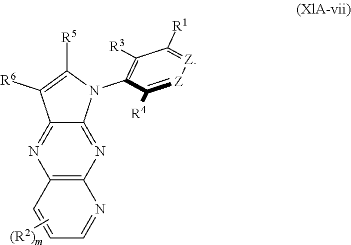

In some embodiments, the Myt1 inhibitor is of formula (XIB):

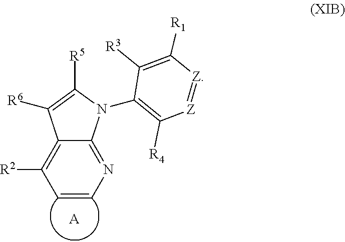

In some embodiments, the Myt1 inhibitor is enriched for the atropisomer of formula (XIB-i):

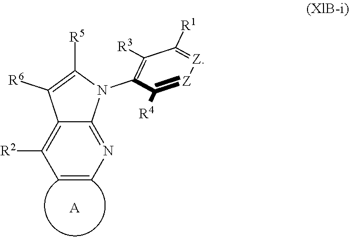

In some embodiments, the Myt1 inhibitor is of formula (XIB-ii):

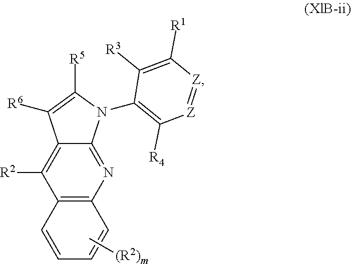

where m is 0, 1, 2, 3, or 4.

In some embodiments, the Myt1 inhibitor is enriched for the atropisomer of formula (XIB-iii):

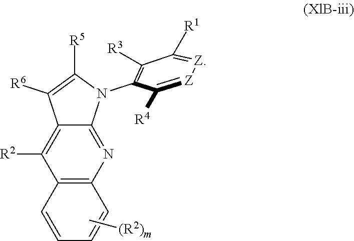

In some embodiments, the Myt1 inhibitor is of formula (XIB-iv):

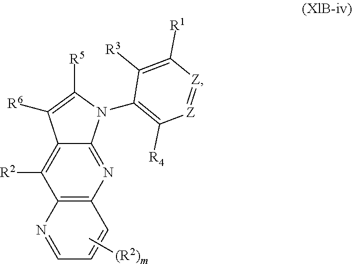

where m is 0, 1, 2, or 3.

In some embodiments, the Myt1 inhibitor is enriched for the atropisomer of formula (XIB-v):

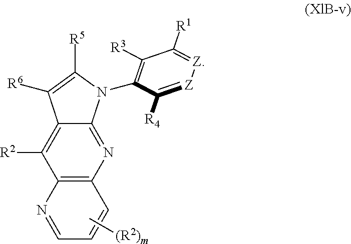

In some embodiments, the Myt1 inhibitor is of formula (XIB-vi):

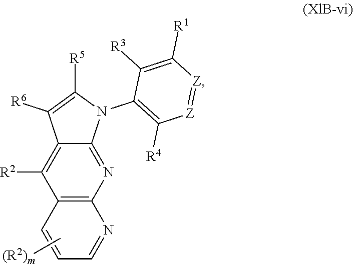

where m is 0, 1, 2, or 3.

In some embodiments, the Myt1 inhibitor is enriched for the atropisomer of formula (XIB-vii):

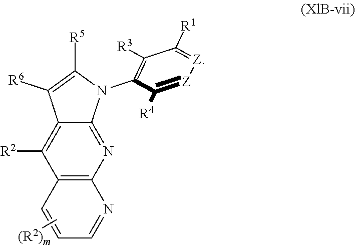

In some embodiments, the Myt1 inhibitor is of formula (XIC):

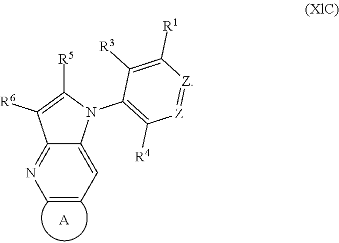

In some embodiments, the Myt1 inhibitor is enriched for the atropisomer of formula (XIC-i):

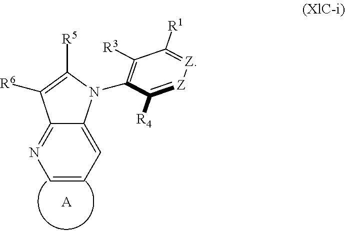

In some embodiments, the Myt1 inhibitor is of formula (XIC-ii):

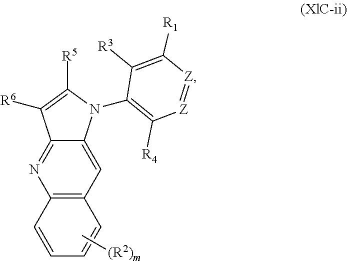

where m is 0, 1, 2, 3, or 4.

In some embodiments, the Myt1 inhibitor is enriched for the atropisomer of formula (XIC-iii):

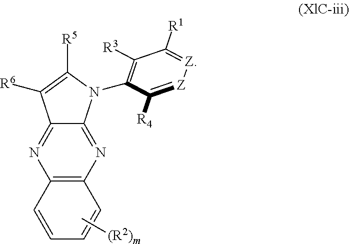

In some embodiments, the Myt1 inhibitor is of formula (XIC-iv):

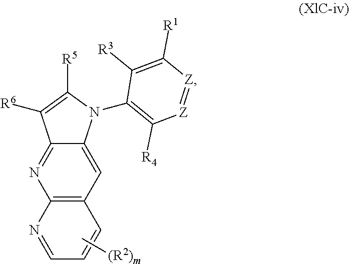

where m is 0, 1, 2, or 3.

In some embodiments, the Myt1 inhibitor is enriched for the atropisomer of formula (XIC-v):

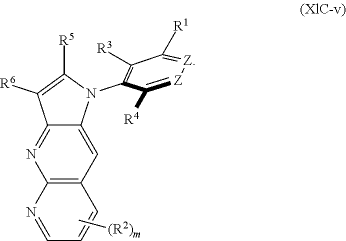

In some embodiments, the Myt1 inhibitor is of formula (XIC-vi):

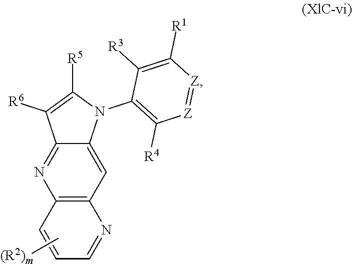

where m is 0, 1, 2, or 3.

In some embodiments, the Myt1 inhibitor is enriched for the atropisomer of formula (XIC-vii):

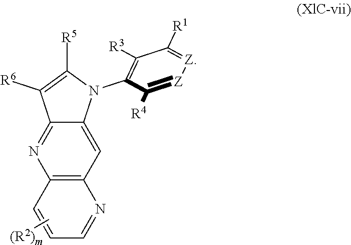

In some embodiments, the Myt1 inhibitor is of formula (XIC-viii):

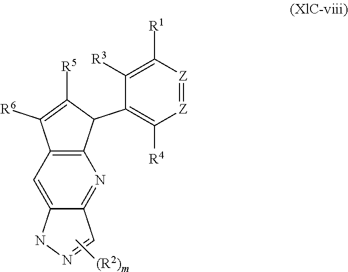

In some embodiments, R² and R^{2A}, together with the atoms to which they are attached, combine to form ring A, and R^{2B} is absent or hydrogen.

In some embodiments, the Myt1 inhibitor is of formula (XII):

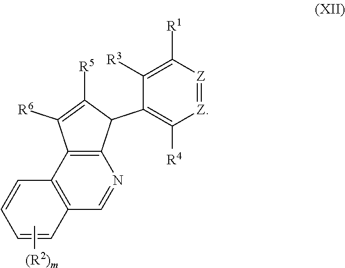

The Myt1 inhibitor may be, e.g., a compound listed in Table 1 below or a pharmaceutically acceptable salt thereof.

TABLE 1
Exemplary Myt1 Inhibitors

| | |
|---|---|
| 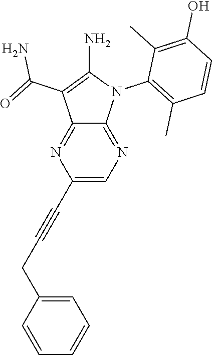 [SMILES: Cc1ccc(O)c(C)c1-n1c(N)c(C(N)=O)c2nc(C#CCc3ccccc3)cnc21] | 1 |
| 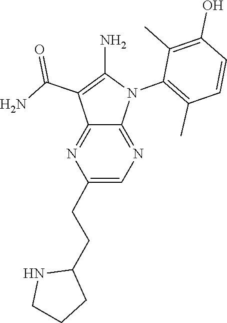 [SMILES: Cc1ccc(O)c(C)c1-n1c(N)c(C(N)=O)c2nc(CCC3CCCN3)cnc21] | 2 |
| 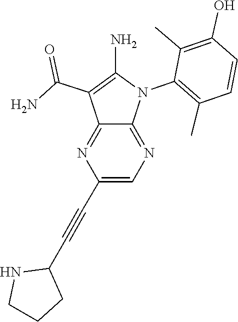 [SMILES: Cc1ccc(O)c(C)c1-n1c(N)c(C(N)=O)c2nc(C#CC3CCCN3)cnc21] | 3 |
| 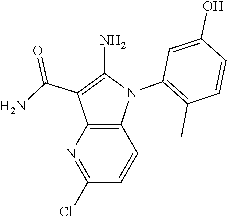 [SMILES: Cc1ccc(O)cc1-n1c(N)c(C(N)=O)c2nc(Cl)ccc21] | 4 |
| 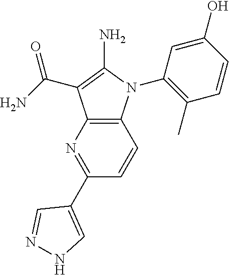 [SMILES: Cc1ccc(O)cc1-n1c(N)c(C(N)=O)c2nc(-c3cn[nH]c3)ccc21] | 5 |
| 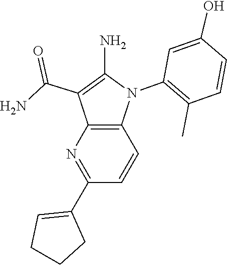 [SMILES: Cc1ccc(O)cc1-n1c(N)c(C(N)=O)c2nc(C3=CCCC3)ccc21] | 6 |
| 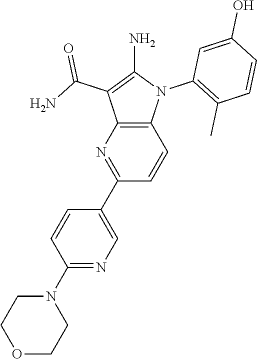 [SMILES: Cc1ccc(O)cc1-n1c(N)c(C(N)=O)c2nc(-c3ccc(N4CCOCC4)nc3)ccc21] | 7 |
| 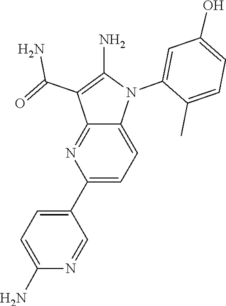 [SMILES: Cc1ccc(O)cc1-n1c(N)c(C(N)=O)c2nc(-c3ccc(N)nc3)ccc21] | 8 |
| 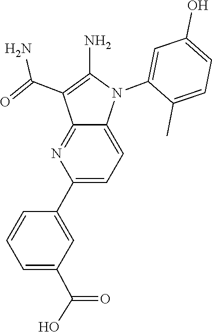 [SMILES: Cc1ccc(O)cc1-n1c(N)c(C(N)=O)c2nc(-c3cccc(C(=O)O)c3)ccc21] | 9 |
| 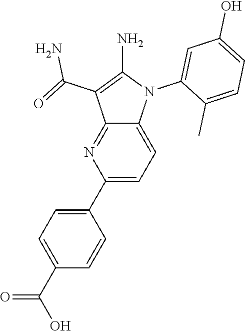 [SMILES: Cc1ccc(O)cc1-n1c(N)c(C(N)=O)c2nc(-c3ccc(C(=O)O)cc3)ccc21] | 10 |
| 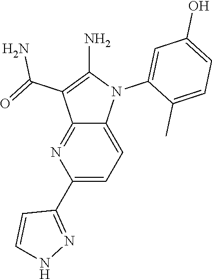 [SMILES: Cc1ccc(O)cc1-n1c(N)c(C(N)=O)c2nc(-c3cc[nH]n3)ccc21] | 11 |
| 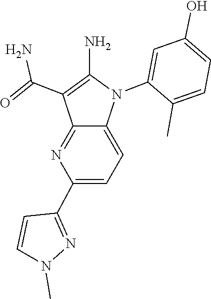 [SMILES: Cc1ccc(O)cc1-n1c(N)c(C(N)=O)c2nc(-c3ccn(C)n3)ccc21] | 12 |
| 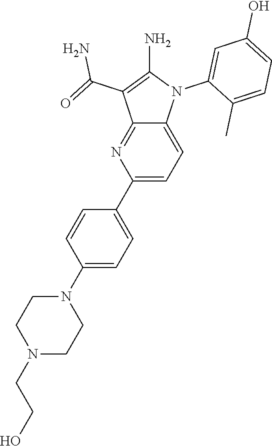 [SMILES: Cc1ccc(O)cc1-n1c(N)c(C(N)=O)c2nc(-c3ccc(N4CCN(CCO)CC4)cc3)ccc21] | 13 |
| 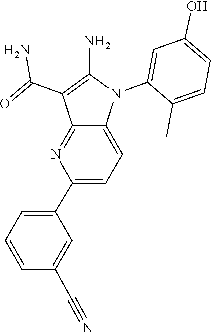 [SMILES: Cc1ccc(O)cc1-n1c(N)c(C(N)=O)c2nc(-c3cccc(C#N)c3)ccc21] | 14 |
| 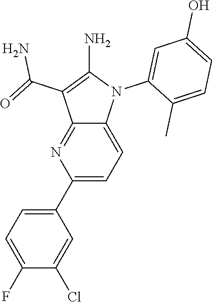 [SMILES: Cc1ccc(O)cc1-n1c(N)c(C(N)=O)c2nc(-c3ccc(F)c(Cl)c3)ccc21] | 15 |
| 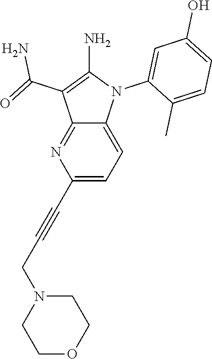 [SMILES: Cc1ccc(O)cc1-n1c(N)c(C(N)=O)c2nc(C#CCN3CCOCC3)ccc21] | 16 |
| 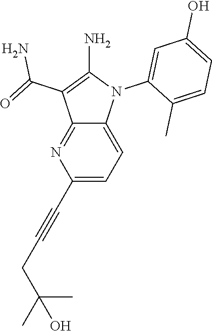 [SMILES: Cc1ccc(O)cc1-n1c(N)c(C(N)=O)c2nc(C#CCC(C)(C)O)ccc21] | 17 |
| 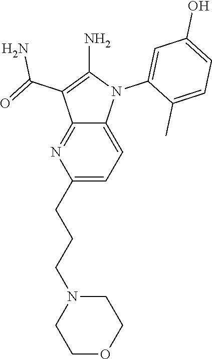 [SMILES: Cc1ccc(O)cc1-n1c(N)c(C(N)=O)c2nc(CCCN3CCOCC3)ccc21] | 18 |
| 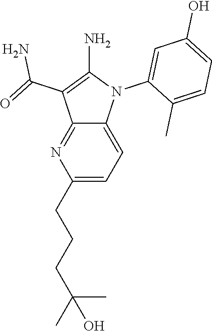 [SMILES: Cc1ccc(O)cc1-n1c(N)c(C(N)=O)c2nc(CCCC(C)(C)O)ccc21] | 19 |
| 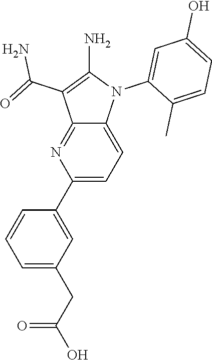 [SMILES: Cc1ccc(O)cc1-n1c(N)c(C(N)=O)c2nc(-c3cccc(CC(=O)O)c3)ccc21] | 20 |
| 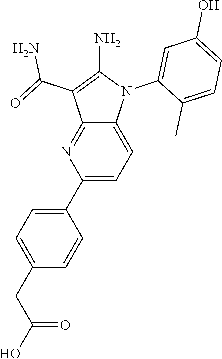 [SMILES: Cc1ccc(O)cc1-n1c(N)c(C(N)=O)c2nc(-c3ccc(CC(=O)O)cc3)ccc21] | 21 |
| 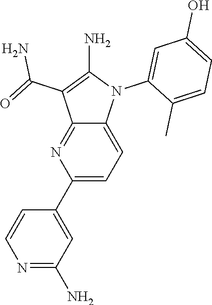 [SMILES: Cc1ccc(O)cc1-n1c(N)c(C(N)=O)c2nc(-c3ccnc(N)c3)ccc21] | 22 |
| 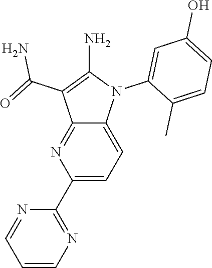 [SMILES: Cc1ccc(O)cc1-n1c(N)c(C(N)=O)c2nc(-c3ncccn3)ccc21] | 23 |
| 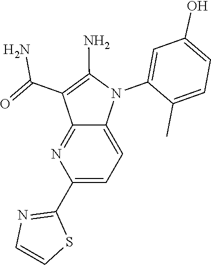 [SMILES: Cc1ccc(O)cc1-n1c(N)c(C(N)=O)c2nc(-c3nccs3)ccc21] | 24 |
| 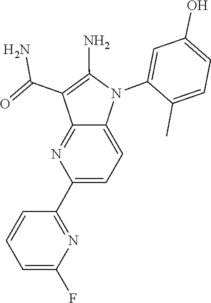 [SMILES: Cc1ccc(O)cc1-n1c(N)c(C(N)=O)c2nc(-c3cccc(F)n3)ccc21] | 25 |
| 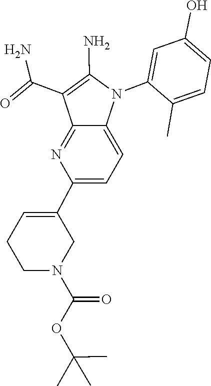 [SMILES: Cc1ccc(O)cc1-n1c(N)c(C(N)=O)c2nc(C3=CCCN(C(=O)OC(C)(C)C)C3)ccc21] | 26 |
| 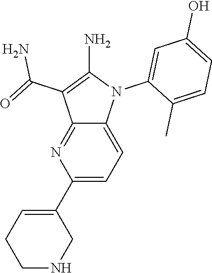 [SMILES: Cc1ccc(O)cc1-n1c(N)c(C(N)=O)c2nc(C3=CCCNC3)ccc21] | 27 |
| 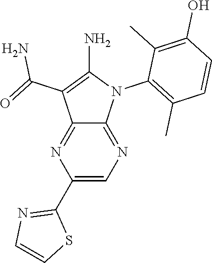 [SMILES: Cc1ccc(O)c(C)c1-n1c(N)c(C(N)=O)c2nc(-c3nccs3)cnc21] | 28 |
| 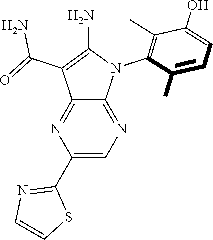 | 29 |
| 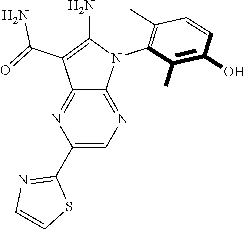 | 30 |
| 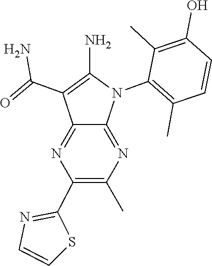 [SMILES: Cc1ccc(O)c(C)c1-n1c(N)c(C(N)=O)c2nc(-c3nccs3)c(C)nc21] | 31 |
| 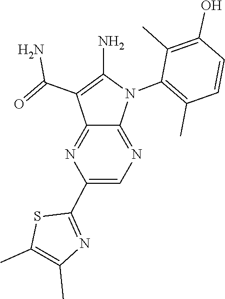 [SMILES: Cc1ccc(O)c(C)c1-n1c(N)c(C(N)=O)c2nc(-c3nc(C)c(C)s3)cnc21] | 32 |
| 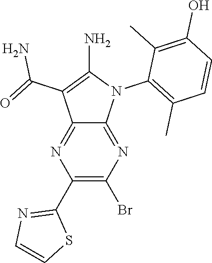 [SMILES: Cc1ccc(O)c(C)c1-n1c(N)c(C(N)=O)c2nc(-c3nccs3)c(Br)nc21] | 33 |
| 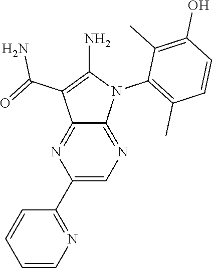 [SMILES: Cc1ccc(O)c(C)c1-n1c(N)c(C(N)=O)c2nc(-c3ccccn3)cnc21] | 34 |
| 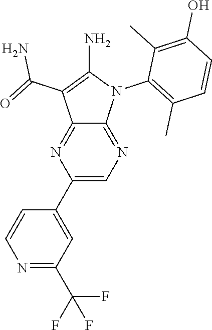 [SMILES: Cc1ccc(O)c(C)c1-n1c(N)c(C(N)=O)c2nc(-c3ccnc(C(F)(F)F)c3)cnc21] | 35 |
| 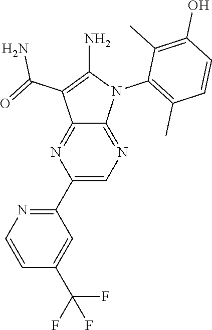 [SMILES: Cc1ccc(O)c(C)c1-n1c(N)c(C(N)=O)c2nc(-c3cc(C(F)(F)F)ccn3)cnc21] | 36 |
| 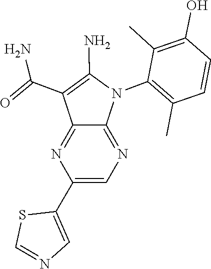 [SMILES: Cc1ccc(O)c(C)c1-n1c(N)c(C(N)=O)c2nc(-c3cncs3)cnc21] | 37 |
| 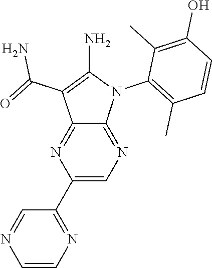 [SMILES: Cc1ccc(O)c(C)c1-n1c(N)c(C(N)=O)c2nc(-c3cnccn3)cnc21] | 38 |
| 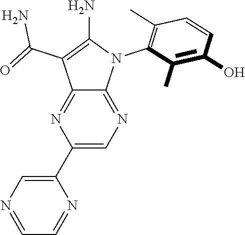 | 39 |
| 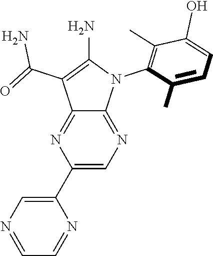 | 40 |
| 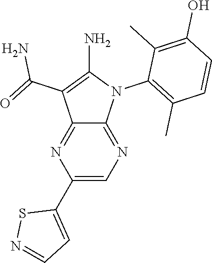 [SMILES: Cc1ccc(O)c(C)c1-n1c(N)c(C(N)=O)c2nc(-c3ccns3)cnc21] | 41 |
| 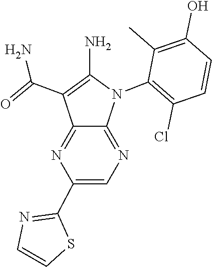 [SMILES: Cc1c(O)ccc(Cl)c1-n1c(N)c(C(N)=O)c2nc(-c3nccs3)cnc21] | 42 |
| 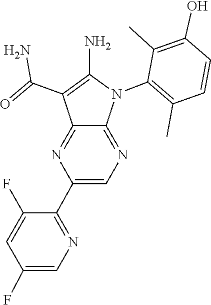 [SMILES: Cc1ccc(O)c(C)c1-n1c(N)c(C(N)=O)c2nc(-c3ncc(F)cc3F)cnc21] | 43 |
| 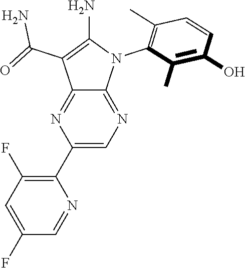 | 44 |
| 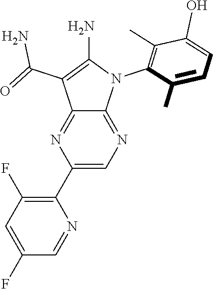 | 45 |
| 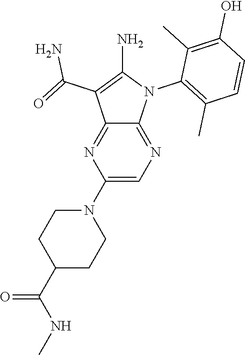 [SMILES: CNC(=O)C1CCN(c2cnc3c(n2)c(C(N)=O)c(N)n3-c2c(C)ccc(O)c2C)CC1] | 46 |
| 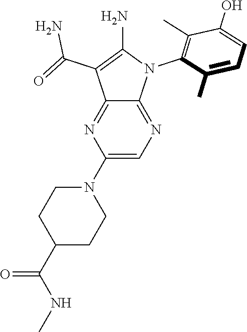 | 47 |
| 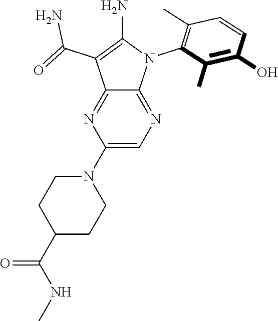 | 48 |
| 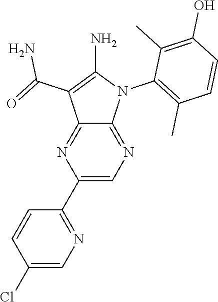 [SMILES: Cc1ccc(O)c(C)c1-n1c(N)c(C(N)=O)c2nc(-c3ccc(Cl)cn3)cnc21] | 49 |
| 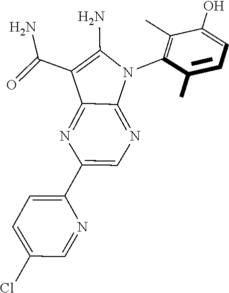 | 50 |
| 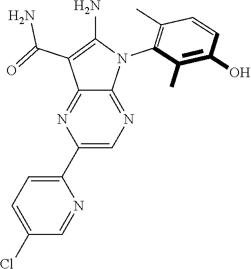 | 51 |
| 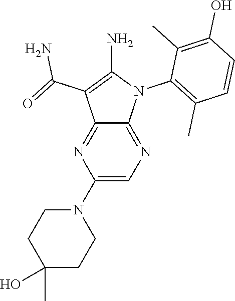 [SMILES: Cc1ccc(O)c(C)c1-n1c(N)c(C(N)=O)c2nc(N3CCC(C)(O)CC3)cnc21] | 52 |
| 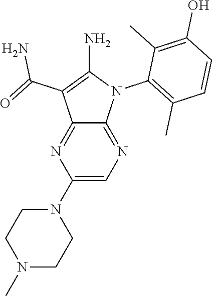 [SMILES: Cc1ccc(O)c(C)c1-n1c(N)c(C(N)=O)c2nc(N3CCN(C)CC3)cnc21] | 53 |
| 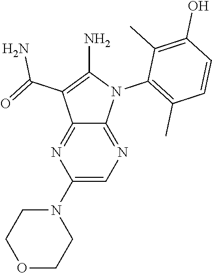 [SMILES: Cc1ccc(O)c(C)c1-n1c(N)c(C(N)=O)c2nc(N3CCOCC3)cnc21] | 54 |
| 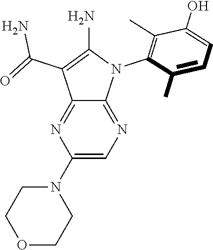 | 55 |
| 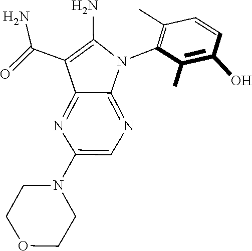 | 56 |
| 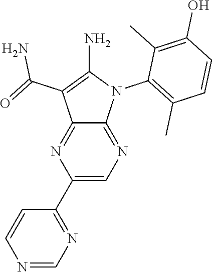 [SMILES: Cc1ccc(O)c(C)c1-n1c(N)c(C(N)=O)c2nc(-c3ccncn3)cnc21] | 57 |
| 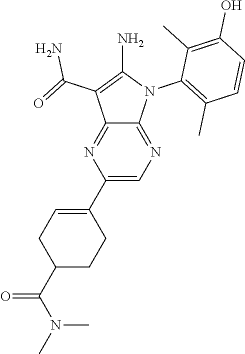 [SMILES: Cc1ccc(O)c(C)c1-n1c(N)c(C(N)=O)c2nc(C3=CCC(C(=O)N(C)C)CC3)cnc21] | 58 |
| 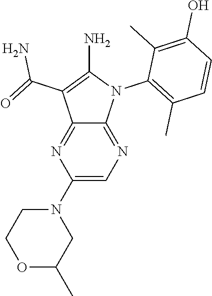 [SMILES: Cc1ccc(O)c(C)c1-n1c(N)c(C(N)=O)c2nc(N3CCOC(C)C3)cnc21] | 59 |
| 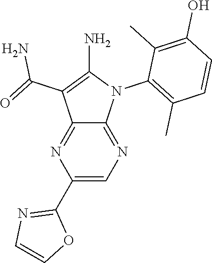 [SMILES: Cc1ccc(O)c(C)c1-n1c(N)c(C(N)=O)c2nc(-c3ncco3)cnc21] | 60 |
| 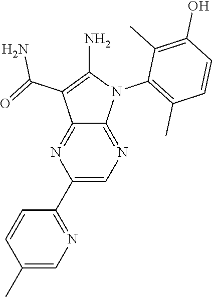 [SMILES: Cc1ccc(-c2cnc3c(n2)c(C(N)=O)c(N)n3-c2c(C)ccc(O)c2C)nc1] | 61 |
| 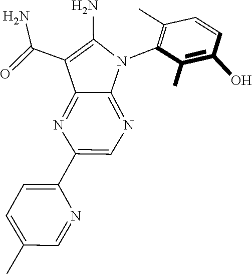 | 62 |
| 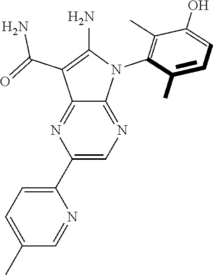 | 63 |
| 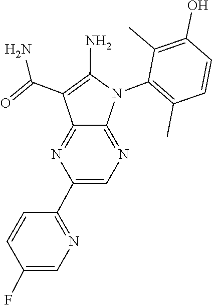 [SMILES: Cc1ccc(O)c(C)c1-n1c(N)c(C(N)=O)c2nc(-c3ccc(F)cn3)cnc21] | 64 |
| 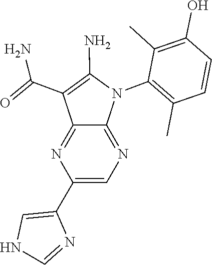 [SMILES: Cc1ccc(O)c(C)c1-n1c(N)c(C(N)=O)c2nc(-c3c[nH]cn3)cnc21] | 65 |
| 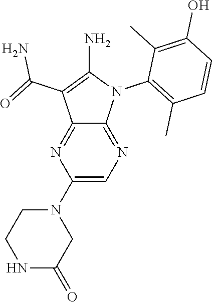 [SMILES: Cc1ccc(O)c(C)c1-n1c(N)c(C(N)=O)c2nc(N3CCNC(=O)C3)cnc21] | 66 |
| 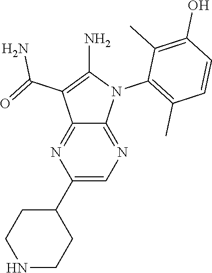 [SMILES: Cc1ccc(O)c(C)c1-n1c(N)c(C(N)=O)c2nc(C3CCNCC3)cnc21] | 67 |
| 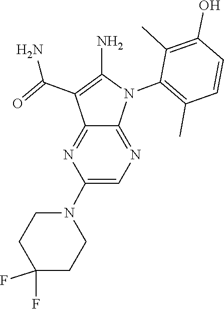 [SMILES: Cc1ccc(O)c(C)c1-n1c(N)c(C(N)=O)c2nc(N3CCC(F)(F)CC3)cnc21] | 68 |
| 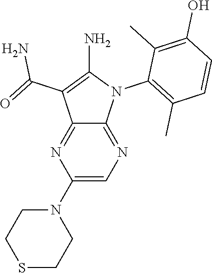 [SMILES: Cc1ccc(O)c(C)c1-n1c(N)c(C(N)=O)c2nc(N3CCSCC3)cnc21] | 69 |
| 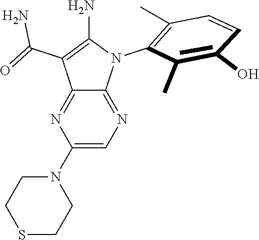 | 70 |
| 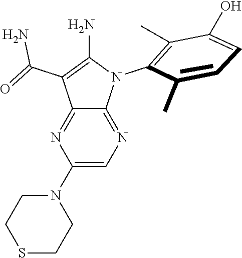 | 71 |
| 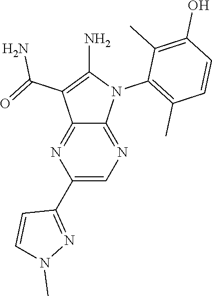 [SMILES: Cc1ccc(O)c(C)c1-n1c(N)c(C(N)=O)c2nc(-c3ccn(C)n3)cnc21] | 72 |
| 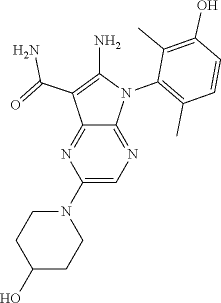 [SMILES: Cc1ccc(O)c(C)c1-n1c(N)c(C(N)=O)c2nc(N3CCC(O)CC3)cnc21] | 73 |
| 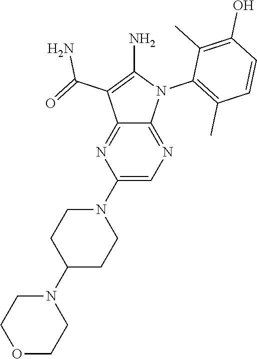 [SMILES: Cc1ccc(O)c(C)c1-n1c(N)c(C(N)=O)c2nc(N3CCC(N4CCOCC4)CC3)cnc21] | 74 |
| 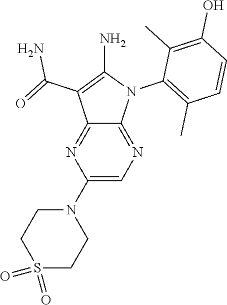 [SMILES: Cc1ccc(O)c(C)c1-n1c(N)c(C(N)=O)c2nc(N3CCS(=O)(=O)CC3)cnc21] | 75 |
| 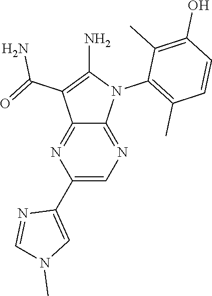 [SMILES: Cc1ccc(O)c(C)c1-n1c(N)c(C(N)=O)c2nc(-c3cn(C)cn3)cnc21] | 76 |
| 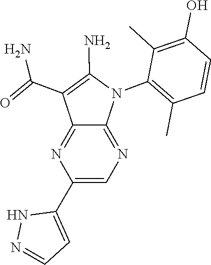 [SMILES: Cc1ccc(O)c(C)c1-n1c(N)c(C(N)=O)c2nc(-c3ccn[nH]3)cnc21] | 77 |
| 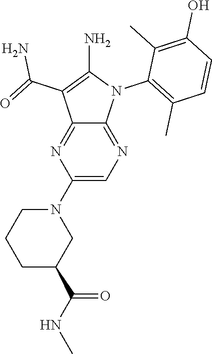 [SMILES: CNC(=O)[C@H]1CCCN(c2cnc3c(n2)c(C(N)=O)c(N)n3-c2c(C)ccc(O)c2C)C1] | 78 |
| 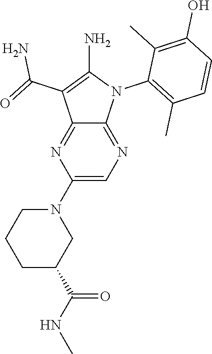 [SMILES: CNC(=O)[C@@H]1CCCN(c2cnc3c(n2)c(C(N)=O)c(N)n3-c2c(C)ccc(O)c2C)C1] | 79 |
| 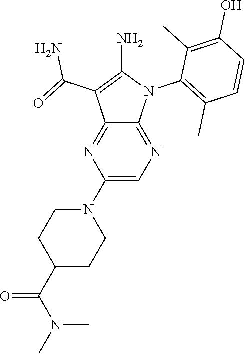 [SMILES: Cc1ccc(O)c(C)c1-n1c(N)c(C(N)=O)c2nc(N3CCC(C(=O)N(C)C)CC3)cnc21] | 80 |
| 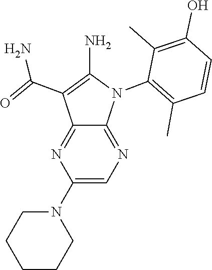 [SMILES: Cc1ccc(O)c(C)c1-n1c(N)c(C(N)=O)c2nc(N3CCCCC3)cnc21] | 81 |
| 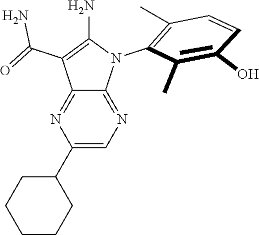 | 82 |
| 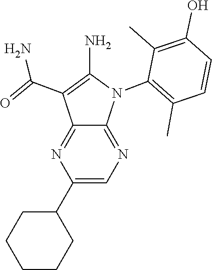 [SMILES: Cc1ccc(O)c(C)c1-n1c(N)c(C(N)=O)c2nc(C3CCCCC3)cnc21] | 83 |
| 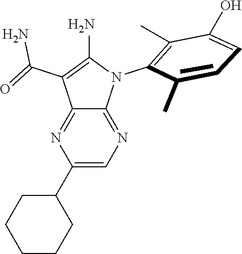 | 84 |
| 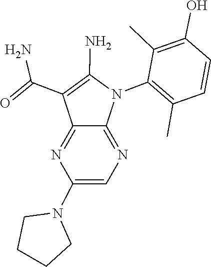 [SMILES: Cc1ccc(O)c(C)c1-n1c(N)c(C(N)=O)c2nc(N3CCCC3)cnc21] | 85 |
| 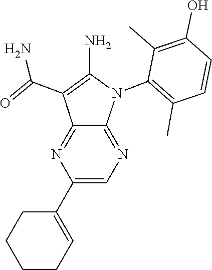 [SMILES: Cc1ccc(O)c(C)c1-n1c(N)c(C(N)=O)c2nc(C3=CCCCC3)cnc21] | 86 |
| 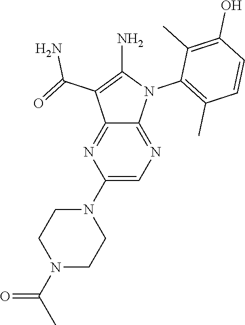 [SMILES: CC(=O)N1CCN(c2cnc3c(n2)c(C(N)=O)c(N)n3-c2c(C)ccc(O)c2C)CC1] | 87 |
| 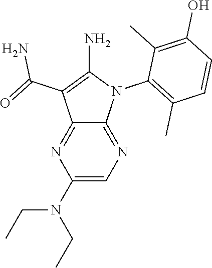 [SMILES: CCN(CC)c1cnc2c(n1)c(C(N)=O)c(N)n2-c1c(C)ccc(O)c1C] | 88 |
| 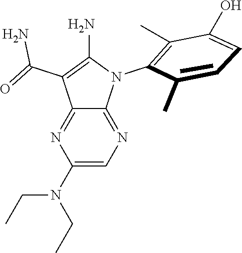 | 89 |
| 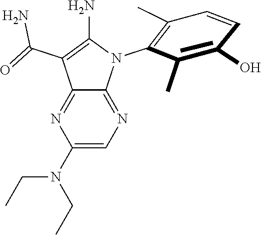 | 90 |
| 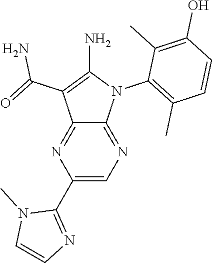 [SMILES: Cc1ccc(O)c(C)c1-n1c(N)c(C(N)=O)c2nc(-c3nccn3C)cnc21] | 91 |
| 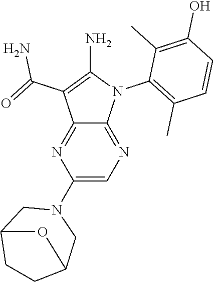 [SMILES: Cc1ccc(O)c(C)c1-n1c(N)c(C(N)=O)c2nc(N3CC4CCC(C3)O4)cnc21] | 92 |
| 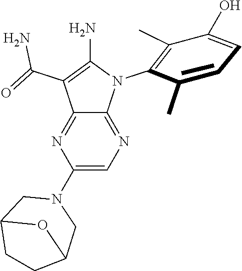 | 93 |
| 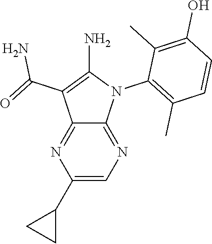 [SMILES: Cc1ccc(O)c(C)c1-n1c(N)c(C(N)=O)c2nc(C3CC3)cnc21] | 94 |
| 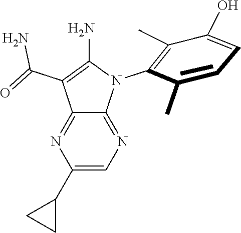 | 95 |
| 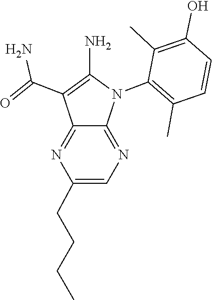 [SMILES: CCCCc1cnc2c(n1)c(C(N)=O)c(N)n2-c1c(C)ccc(O)c1C] | 96 |
| 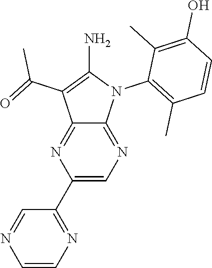 [SMILES: CC(=O)c1c(N)n(-c2c(C)ccc(O)c2C)c2ncc(-c3cnccn3)nc12] | 97 |
| 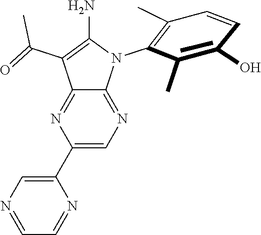 | 98 |
| 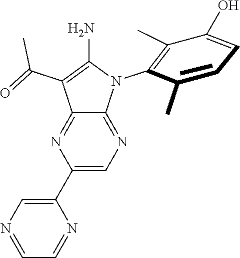 | 99 |
| 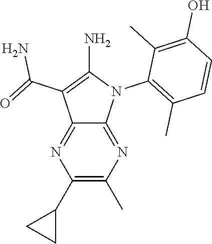 [SMILES: Cc1ccc(O)c(C)c1-n1c(N)c(C(N)=O)c2nc(C3CC3)c(C)nc21] | 100 |
| 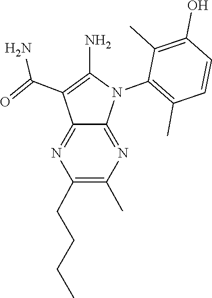 [SMILES: CCCCc1nc2c(C(N)=O)c(N)n(-c3c(C)ccc(O)c3C)c2nc1C] | 101 |
| 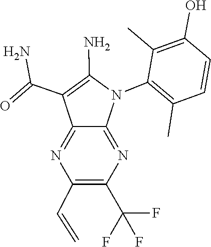 [SMILES: C=Cc1nc2c(C(N)=O)c(N)n(-c3c(C)ccc(O)c3C)c2nc1C(F)(F)F] | 102 |
| 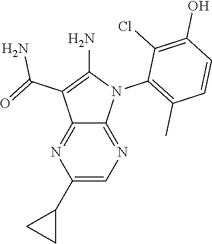 [SMILES: Cc1ccc(O)c(Cl)c1-n1c(N)c(C(N)=O)c2nc(C3CC3)cnc21] | 104 |
| 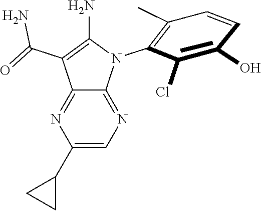 | 105 |
| 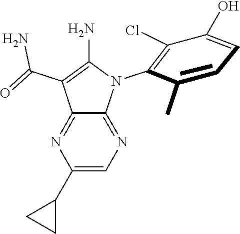 | 106 |
| 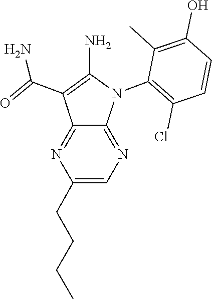 [SMILES: CCCCc1cnc2c(n1)c(C(N)=O)c(N)n2-c1c(Cl)ccc(O)c1C] | 107 |
| 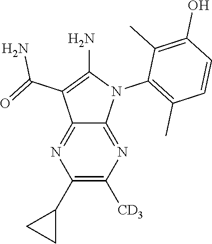 [SMILES: [2H]C([2H])([2H])c1nc2c(nc1C1CC1)c(C(N)=O)c(N)n2-c1c(C)ccc(O)c1C] | 110 |
| 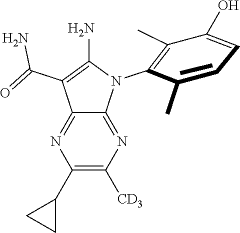 | 111 |
| 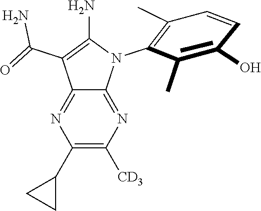 | 112 |
| 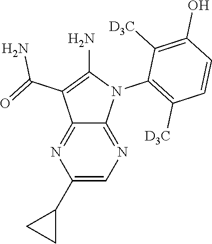 [SMILES: [2H]C([2H])([2H])c1ccc(O)c(C([2H])([2H])[2H])c1-n1c(N)c(C(N)=O)c2nc(C3CC3)cnc21] | 113 |
| 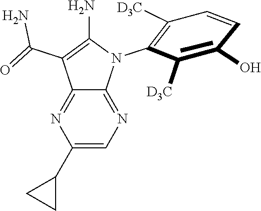 | 114 |
| 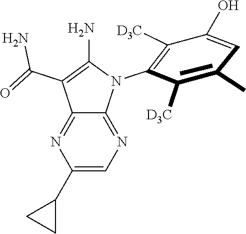 | 115 |
| 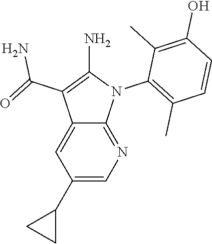 [SMILES: Cc1ccc(O)c(C)c1-n1c(N)c(C(N)=O)c2cc(C3CC3)cnc21] | 116 |
| 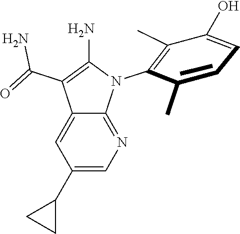 | 117 |
| 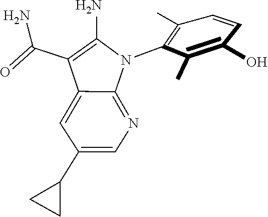 | 118 |
| 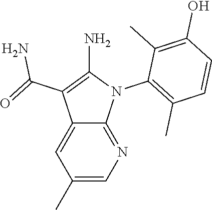 [SMILES: Cc1cnc2c(c1)c(C(N)=O)c(N)n2-c1c(C)ccc(O)c1C] | 119 |
| 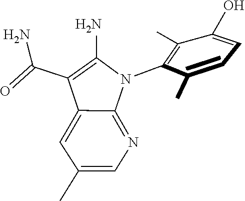 | 120 |
| 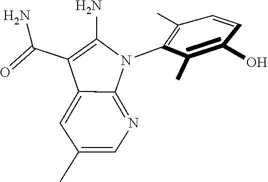 | 121 |
| 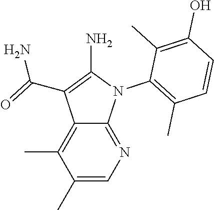 [SMILES: Cc1ccc(O)c(C)c1-n1c(N)c(C(N)=O)c2c(C)c(C)cnc21] | 125 |
| 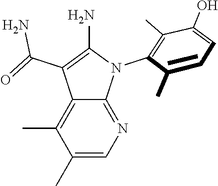 | 126 |
| 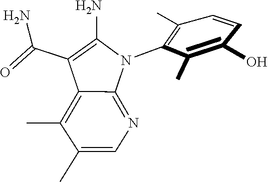 | 127 |
| 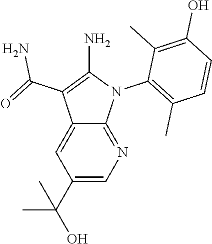 [SMILES: Cc1ccc(O)c(C)c1-n1c(N)c(C(N)=O)c2cc(C(C)(C)O)cnc21] | 131 |
| 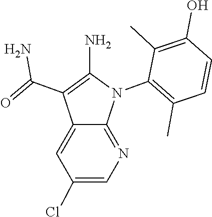 [SMILES: Cc1ccc(O)c(C)c1-n1c(N)c(C(N)=O)c2cc(Cl)cnc21] | 132 |
| 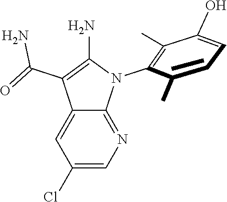 | 133 |
| 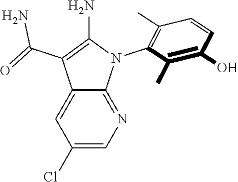 | 134 |
| 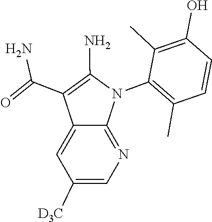 [SMILES: [2H]C([2H])([2H])c1cnc2c(c1)c(C(N)=O)c(N)n2-c1c(C)ccc(O)c1C] | 135 |
| 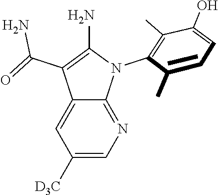 | 136 |
| 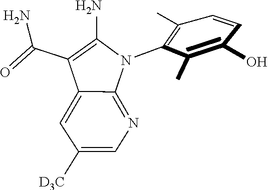 | 137 |
| 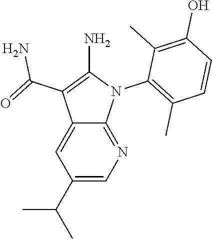 [SMILES: Cc1ccc(O)c(C)c1-n1c(N)c(C(N)=O)c2cc(C(C)C)cnc21] | 138 |
| 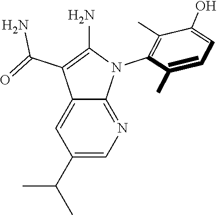 | 139 |
| 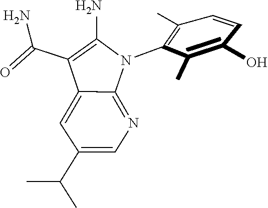 | 140 |
| 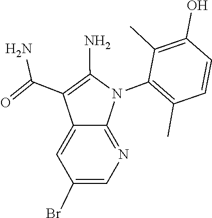 [SMILES: Cc1ccc(O)c(C)c1-n1c(N)c(C(N)=O)c2cc(Br)cnc21] | 141 |
| 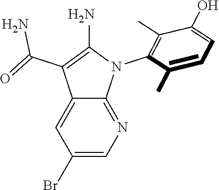 | 142 |
| 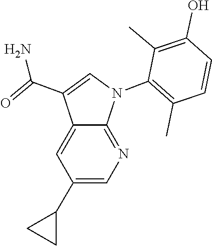 [SMILES: Cc1ccc(O)c(C)c1-n1cc(C(N)=O)c2cc(C3CC3)cnc21] | 143 |
| 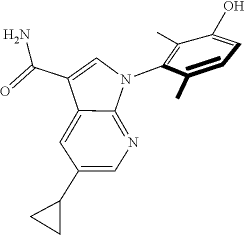 | 144 |
| 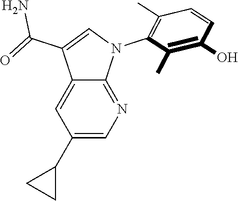 | 145 |
| 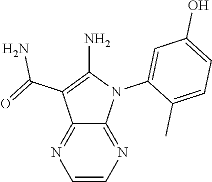 [SMILES: Cc1ccc(O)cc1-n1c(N)c(C(N)=O)c2nccnc21] | 146 |
| 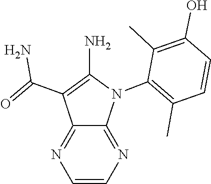 [SMILES: Cc1ccc(O)c(C)c1-n1c(N)c(C(N)=O)c2nccnc21] | 147 |
| 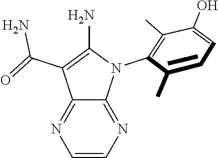 | 148 |
| 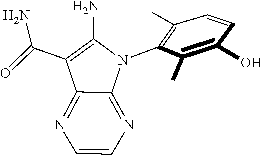 | 149 |
| 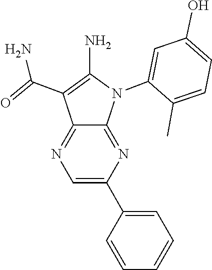 [SMILES: Cc1ccc(O)cc1-n1c(N)c(C(N)=O)c2ncc(-c3ccccc3)nc21] | 150 |
| 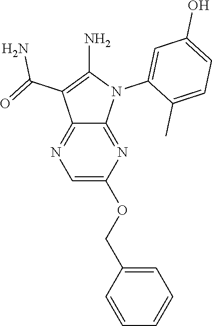 [SMILES: Cc1ccc(O)cc1-n1c(N)c(C(N)=O)c2ncc(OCc3ccccc3)nc21] | 151 |
| 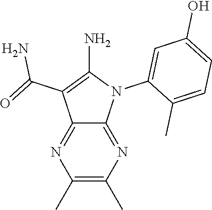 [SMILES: Cc1ccc(O)cc1-n1c(N)c(C(N)=O)c2nc(C)c(C)nc21] | 152 |
| 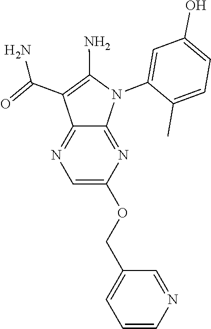 [SMILES: Cc1ccc(O)cc1-n1c(N)c(C(N)=O)c2ncc(OCc3cccnc3)nc21] | 153 |
| 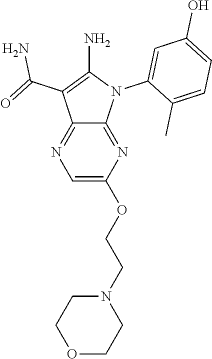 [SMILES: Cc1ccc(O)cc1-n1c(N)c(C(N)=O)c2ncc(OCCN3CCOCC3)nc21] | 154 |
| 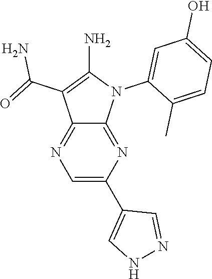 [SMILES: Cc1ccc(O)cc1-n1c(N)c(C(N)=O)c2ncc(-c3cn[nH]c3)nc21] | 155 |
| 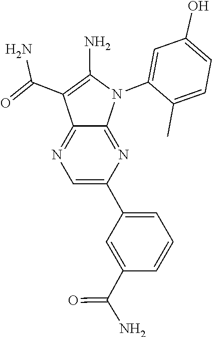 [SMILES: Cc1ccc(O)cc1-n1c(N)c(C(N)=O)c2ncc(-c3cccc(C(N)=O)c3)nc21] | 156 |
| 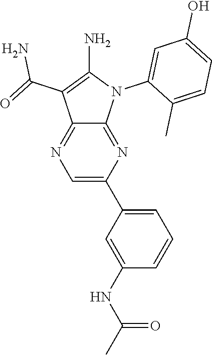 [SMILES: CC(=O)Nc1cccc(-c2cnc3c(C(N)=O)c(N)n(-c4cc(O)ccc4C)c3n2)c1] | 157 |
| 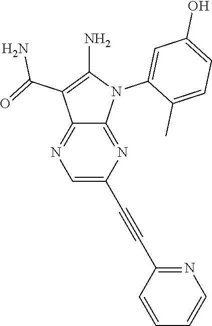 [SMILES: Cc1ccc(O)cc1-n1c(N)c(C(N)=O)c2ncc(C#Cc3ccccn3)nc21] | 158 |
| 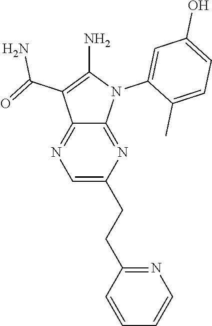 [SMILES: Cc1ccc(O)cc1-n1c(N)c(C(N)=O)c2ncc(CCc3ccccn3)nc21] | 159 |
| 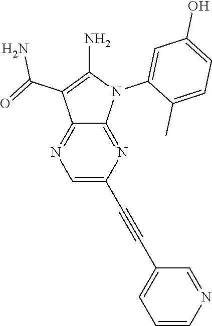 [SMILES: Cc1ccc(O)cc1-n1c(N)c(C(N)=O)c2ncc(C#Cc3cccnc3)nc21] | 160 |
| 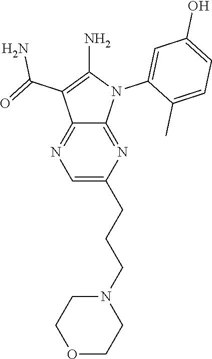 [SMILES: Cc1ccc(O)cc1-n1c(N)c(C(N)=O)c2ncc(CCCN3CCOCC3)nc21] | 161 |
| 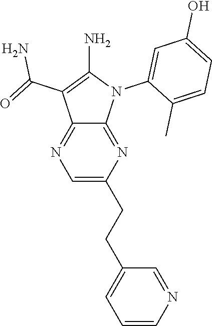 [SMILES: Cc1ccc(O)cc1-n1c(N)c(C(N)=O)c2ncc(CCc3cccnc3)nc21] | 162 |
| 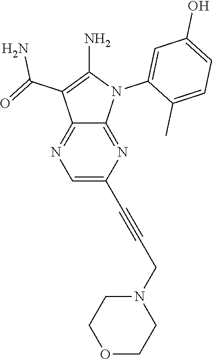 [SMILES: Cc1ccc(O)cc1-n1c(N)c(C(N)=O)c2ncc(C#CCN3CCOCC3)nc21] | 163 |
| 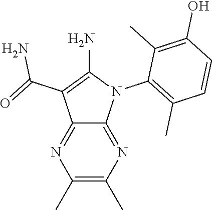 [SMILES: Cc1ccc(O)c(C)c1-n1c(N)c(C(N)=O)c2nc(C)c(C)nc21] | 164 |
| 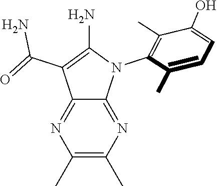 | 165 |
| 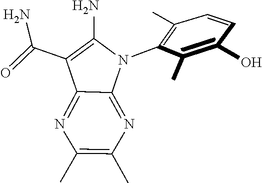 | 166 |
| 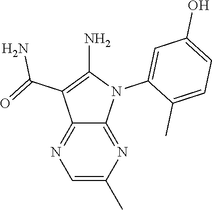 [SMILES: Cc1cnc2c(C(N)=O)c(N)n(-c3cc(O)ccc3C)c2n1] | 167 |
| 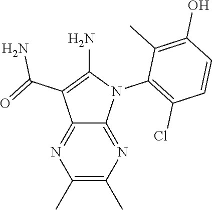 [SMILES: Cc1nc2c(C(N)=O)c(N)n(-c3c(Cl)ccc(O)c3C)c2nc1C] | 168 |
| 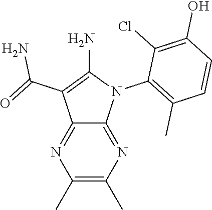 [SMILES: Cc1ccc(O)c(Cl)c1-n1c(N)c(C(N)=O)c2nc(C)c(C)nc21] | 170 |
| 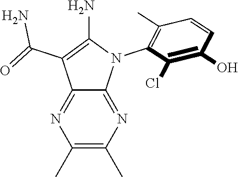 | 171 |
| 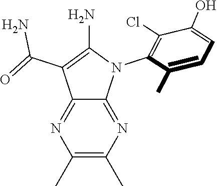 | 172 |
| 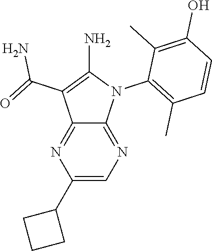 [SMILES: Cc1ccc(O)c(C)c1-n1c(N)c(C(N)=O)c2nc(C3CCC3)cnc21] | 173 |
| 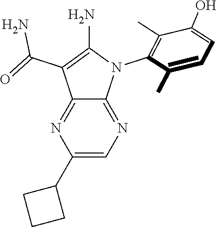 | 174 |
| 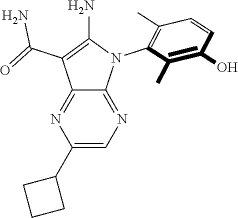 | 175 |
| 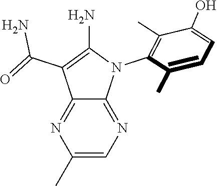 | 176 |
| 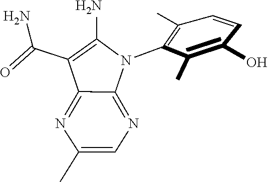 | 177 |
| 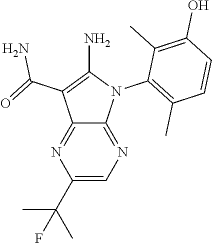 [SMILES: Cc1ccc(O)c(C)c1-n1c(N)c(C(N)=O)c2nc(C(C)(C)F)cnc21] | 178 |
| 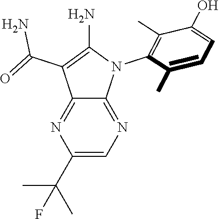 | 179 |
| 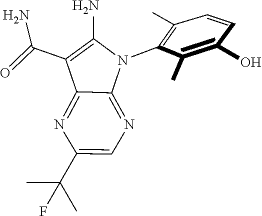 | 180 |
| 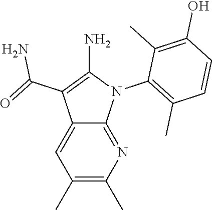 [SMILES: Cc1cc2c(C(N)=O)c(N)n(-c3c(C)ccc(O)c3C)c2nc1C] | 181 |
| 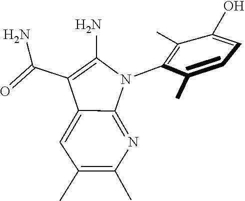 | 182 |
| 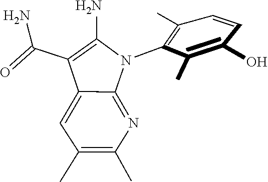 | 183 |
| 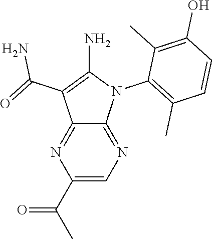 [SMILES: CC(=O)c1cnc2c(n1)c(C(N)=O)c(N)n2-c1c(C)ccc(O)c1C] | 184 |
| 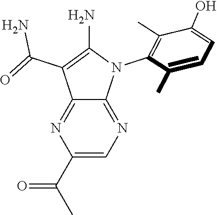 | 185 |
| 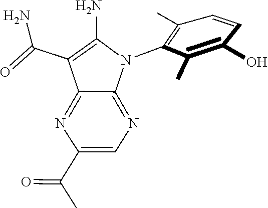 | 186 |
| 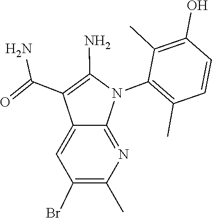 [SMILES: Cc1ccc(O)c(C)c1-n1c(N)c(C(N)=O)c2cc(Br)c(C)nc21] | 187 |
| 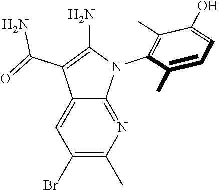 | 188 |
| 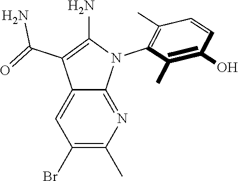 | 189 |
| 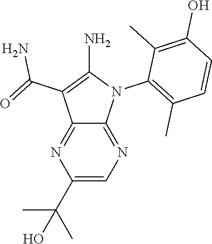 [SMILES: Cc1ccc(O)c(C)c1-n1c(N)c(C(N)=O)c2nc(C(C)(C)O)cnc21] | 190 |
| 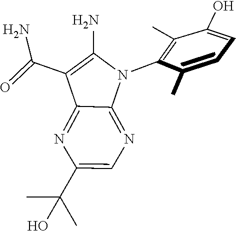 | 191 |
| 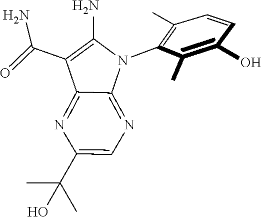 | 192 |
| 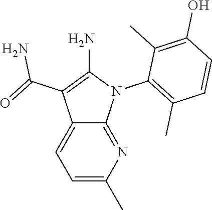 [SMILES: Cc1ccc2c(C(N)=O)c(N)n(-c3c(C)ccc(O)c3C)c2n1] | 193 |
| 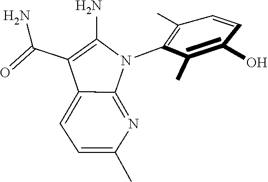 | 194 |
| 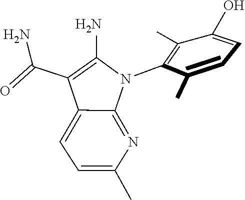 | 195 |
| 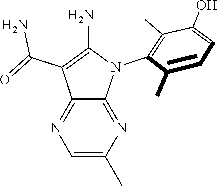 | 196 |
| 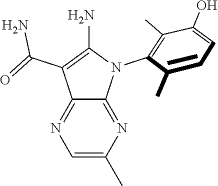 | 197 |
| 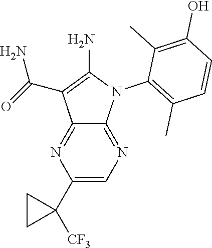 [SMILES: Cc1ccc(O)c(C)c1-n1c(N)c(C(N)=O)c2nc(C3(C(F)(F)F)CC3)cnc21] | 198 |
| 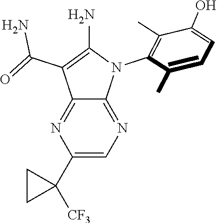 | 199 |
| 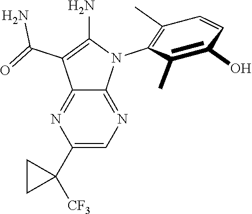 | 200 |
| 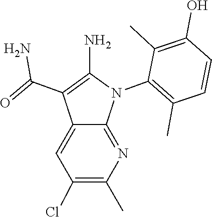 [SMILES: Cc1ccc(O)c(C)c1-n1c(N)c(C(N)=O)c2cc(Cl)c(C)nc21] | 201 |
| 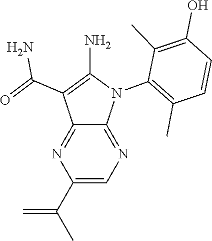 [SMILES: C=C(C)c1cnc2c(n1)c(C(N)=O)c(N)n2-c1c(C)ccc(O)c1C] | 202 |
| 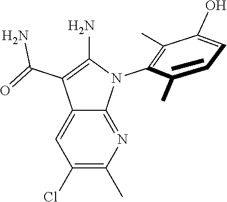 | 203 |
| 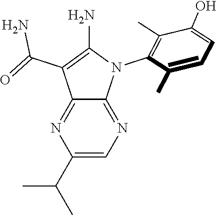 | 204 |
| 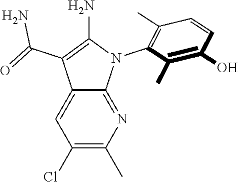 | 205 |
| 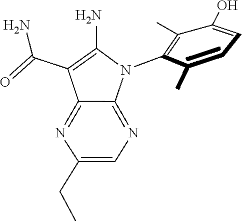 | 206 |
| 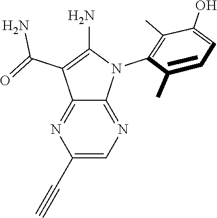 | 208 |
| 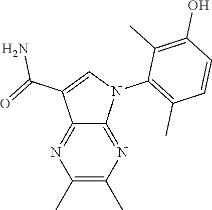 [SMILES: Cc1ccc(O)c(C)c1-n1cc(C(N)=O)c2nc(C)c(C)nc21] | 209 |
| 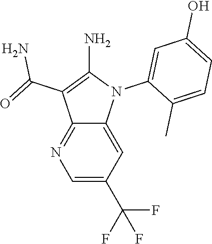 [SMILES: Cc1ccc(O)cc1-n1c(N)c(C(N)=O)c2ncc(C(F)(F)F)cc21] | 212 |
| 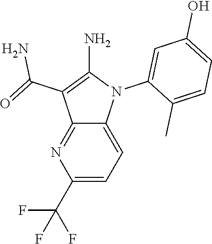 [SMILES: Cc1ccc(O)cc1-n1c(N)c(C(N)=O)c2nc(C(F)(F)F)ccc21] | 213 |
| 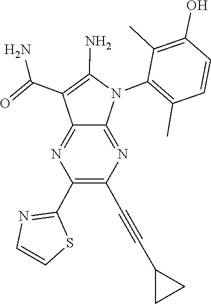 [SMILES: Cc1ccc(O)c(C)c1-n1c(N)c(C(N)=O)c2nc(-c3nccs3)c(C#CC3CC3)nc21] | 214 |
| 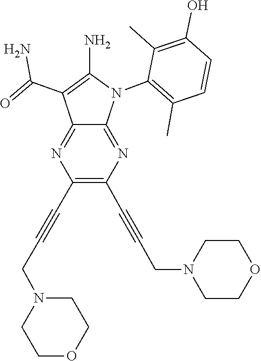 [SMILES: Cc1ccc(O)c(C)c1-n1c(N)c(C(N)=O)c2nc(C#CCN3CCOCC3)c(C#CCN3CCOCC3)nc21] | 215 |
| 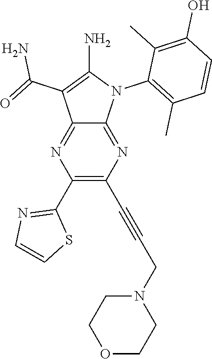 [SMILES: Cc1ccc(O)c(C)c1-n1c(N)c(C(N)=O)c2nc(-c3nccs3)c(C#CCN3CCOCC3)nc21] | 216 |
| 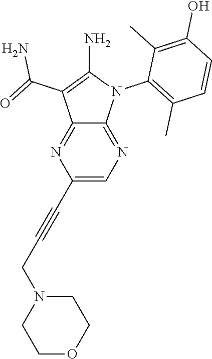 [SMILES: Cc1ccc(O)c(C)c1-n1c(N)c(C(N)=O)c2nc(C#CCN3CCOCC3)cnc21] | 217 |
| 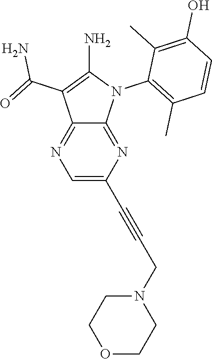 [SMILES: Cc1ccc(O)c(C)c1-n1c(N)c(C(N)=O)c2ncc(C#CCN3CCOCC3)nc21] | 218 |
| 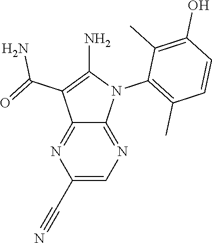 [SMILES: Cc1ccc(O)c(C)c1-n1c(N)c(C(N)=O)c2nc(C#N)cnc21] | 219 |
| 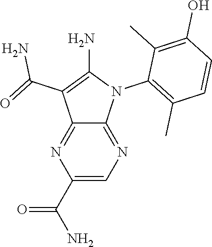 [SMILES: Cc1ccc(O)c(C)c1-n1c(N)c(C(N)=O)c2nc(C(N)=O)cnc21] | 220 |
| 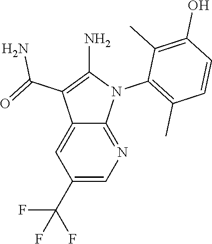 [SMILES: Cc1ccc(O)c(C)c1-n1c(N)c(C(N)=O)c2cc(C(F)(F)F)cnc21] | 221 |
| 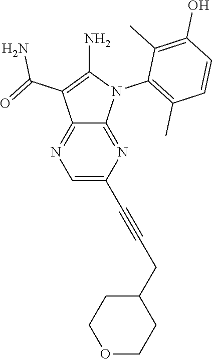 [SMILES: Cc1ccc(O)c(C)c1-n1c(N)c(C(N)=O)c2ncc(C#CCC3CCOCC3)nc21] | 222 |
| 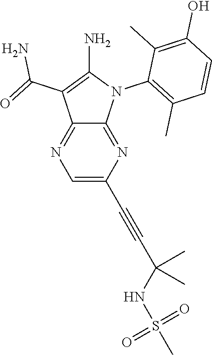 [SMILES: Cc1ccc(O)c(C)c1-n1c(N)c(C(N)=O)c2ncc(C#CC(C)(C)N[S@SP2](C)(=O)=O)nc21] | 223 |
| 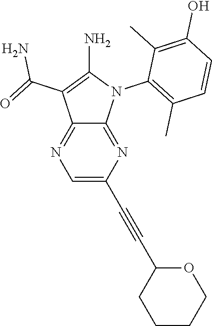 [SMILES: Cc1ccc(O)c(C)c1-n1c(N)c(C(N)=O)c2ncc(C#CC3CCCCO3)nc21] | 224 |
| 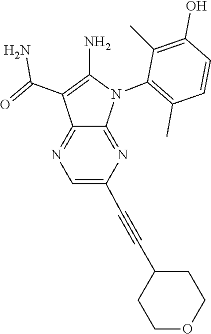 [SMILES: Cc1ccc(O)c(C)c1-n1c(N)c(C(N)=O)c2ncc(C#CC3CCOCC3)nc21] | 225 |
| 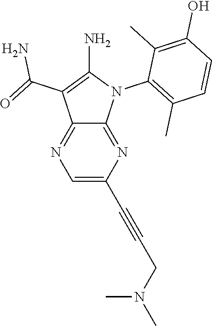 [SMILES: Cc1ccc(O)c(C)c1-n1c(N)c(C(N)=O)c2ncc(C#CCN(C)C)nc21] | 226 |
| 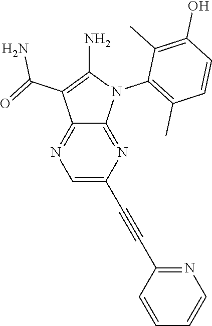 [SMILES: Cc1ccc(O)c(C)c1-n1c(N)c(C(N)=O)c2ncc(C#Cc3ccccn3)nc21] | 227 |
| 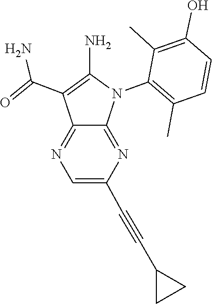 [SMILES: Cc1ccc(O)c(C)c1-n1c(N)c(C(N)=O)c2ncc(C#CC3CC3)nc21] | 228 |
| 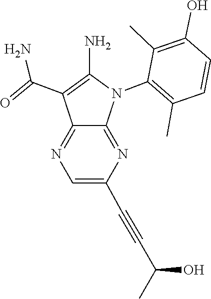 [SMILES: Cc1ccc(O)c(C)c1-n1c(N)c(C(N)=O)c2ncc(C#C[C@H](C)O)nc21] | 229 |
| 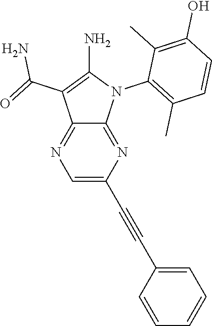 [SMILES: Cc1ccc(O)c(C)c1-n1c(N)c(C(N)=O)c2ncc(C#Cc3ccccc3)nc21] | 230 |
| 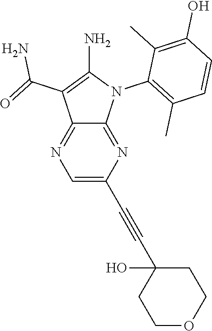 [SMILES: Cc1ccc(O)c(C)c1-n1c(N)c(C(N)=O)c2ncc(C#CC3(O)CCOCC3)nc21] | 231 |
| 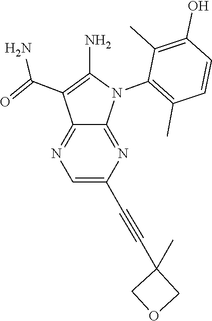 [SMILES: Cc1ccc(O)c(C)c1-n1c(N)c(C(N)=O)c2ncc(C#CC3(C)COC3)nc21] | 232 |
| 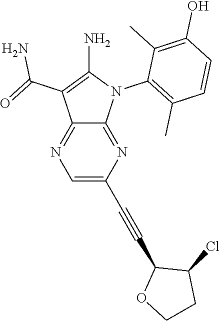 [SMILES: Cc1ccc(O)c(C)c1-n1c(N)c(C(N)=O)c2ncc(C#C[C@@H]3OCC[C@@H]3Cl)nc21] | 233 |
| 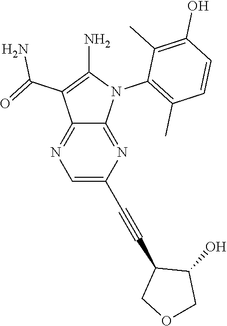 [SMILES: Cc1ccc(O)c(C)c1-n1c(N)c(C(N)=O)c2ncc(C#C[C@@H]3COC[C@H]3O)nc21] | 234 |
| 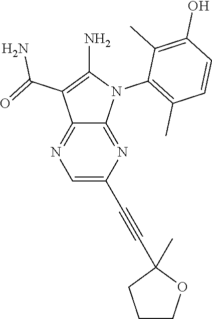 [SMILES: Cc1ccc(O)c(C)c1-n1c(N)c(C(N)=O)c2ncc(C#CC3(C)CCCO3)nc21] | 235 |
| 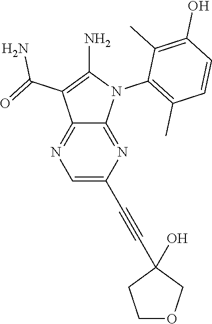 [SMILES: Cc1ccc(O)c(C)c1-n1c(N)c(C(N)=O)c2ncc(C#CC3(O)CCOC3)nc21] | 236 |
| 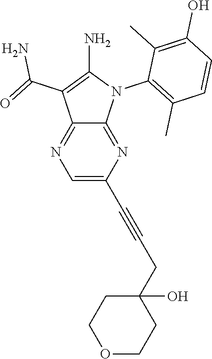 [SMILES: Cc1ccc(O)c(C)c1-n1c(N)c(C(N)=O)c2ncc(C#CCC3(O)CCOCC3)nc21] | 237 |
| 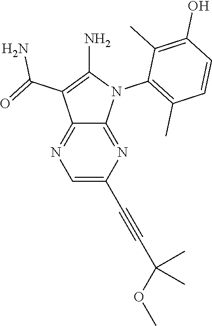 [SMILES: COC(C)(C)C#Cc1cnc2c(C(N)=O)c(N)n(-c3c(C)ccc(O)c3C)c2n1] | 238 |
| 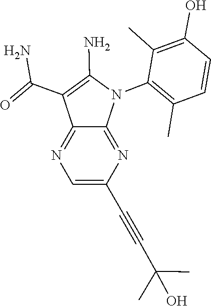 [SMILES: Cc1ccc(O)c(C)c1-n1c(N)c(C(N)=O)c2ncc(C#CC(C)(C)O)nc21] | 239 |
| 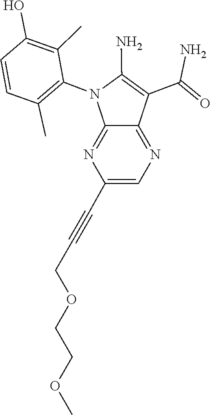 [SMILES: COCCOCC#Cc1cnc2c(C(N)=O)c(N)n(-c3c(C)ccc(O)c3C)c2n1] | 240 |
| 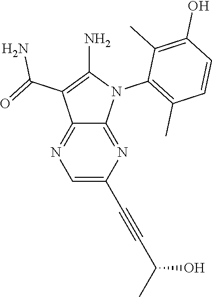 | 241 |
| 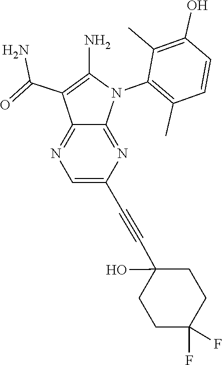 [SMILES: Cc1ccc(O)c(C)c1-n1c(N)c(C(N)=O)c2ncc(C#CC3(O)CCC(F)(F)CC3)nc21] | 242 |
| 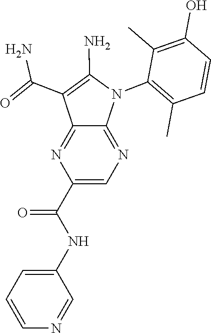 [SMILES: Cc1ccc(O)c(C)c1-n1c(N)c(C(N)=O)c2nc(C(=O)Nc3cccnc3)cnc21] | 243 |
| 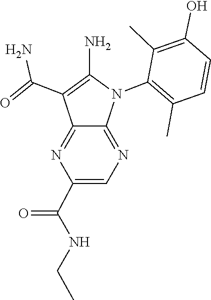 [SMILES: CCNC(=O)c1cnc2c(n1)c(C(N)=O)c(N)n2-c1c(C)ccc(O)c1C] | 244 |
| 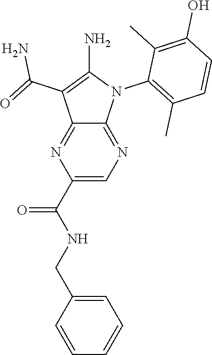 [SMILES: Cc1ccc(O)c(C)c1-n1c(N)c(C(N)=O)c2nc(C(=O)NCc3ccccc3)cnc21] | 245 |
| 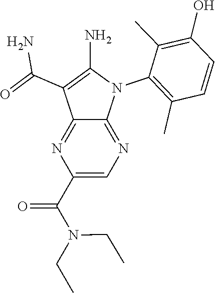 [SMILES: CCN(CC)C(=O)c1cnc2c(n1)c(C(N)=O)c(N)n2-c1c(C)ccc(O)c1C] | 246 |
| 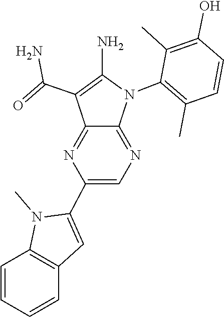 [SMILES: Cc1ccc(O)c(C)c1-n1c(N)c(C(N)=O)c2nc(-c3cc4ccccc4n3C)cnc21] | 247 |
| 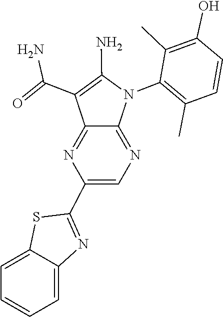 [SMILES: Cc1ccc(O)c(C)c1-n1c(N)c(C(N)=O)c2nc(-c3nc4ccccc4s3)cnc21] | 248 |
| 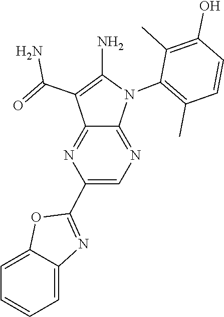 [SMILES: Cc1ccc(O)c(C)c1-n1c(N)c(C(N)=O)c2nc(-c3nc4ccccc4o3)cnc21] | 249 |
| 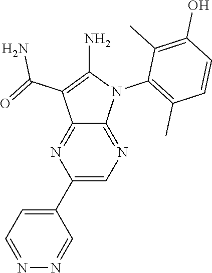 [SMILES: Cc1ccc(O)c(C)c1-n1c(N)c(C(N)=O)c2nc(-c3ccnnc3)cnc21] | 250 |
| 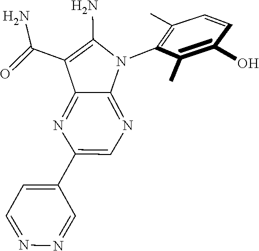 | 251 |
| 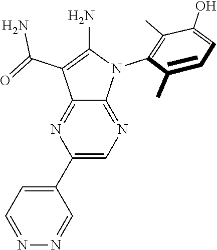 | 252 |
| 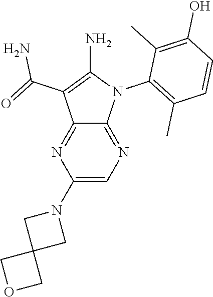 [SMILES: Cc1ccc(O)c(C)c1-n1c(N)c(C(N)=O)c2nc(N3CC4(COC4)C3)cnc21] | 253 |
| 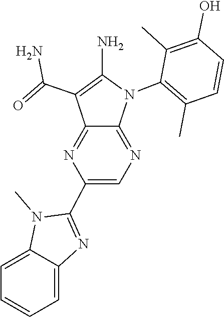 [SMILES: Cc1ccc(O)c(C)c1-n1c(N)c(C(N)=O)c2nc(-c3nc4ccccc4n3C)cnc21] | 254 |
| 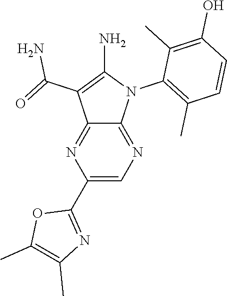 [SMILES: Cc1ccc(O)c(C)c1-n1c(N)c(C(N)=O)c2nc(-c3nc(C)c(C)o3)cnc21] | 255 |
| 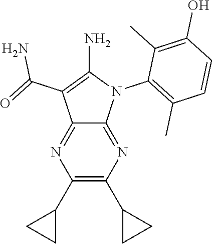 [SMILES: Cc1ccc(O)c(C)c1-n1c(N)c(C(N)=O)c2nc(C3CC3)c(C3CC3)nc21] | 256 |
| 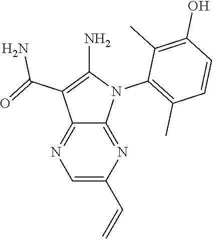 [SMILES: C=Cc1cnc2c(C(N)=O)c(N)n(-c3c(C)ccc(O)c3C)c2n1] | 257 |
| 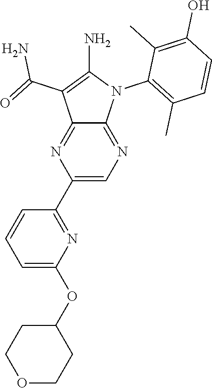 [SMILES: Cc1ccc(O)c(C)c1-n1c(N)c(C(N)=O)c2nc(-c3cccc(OC4CCOCC4)n3)cnc21] | 258 |
| 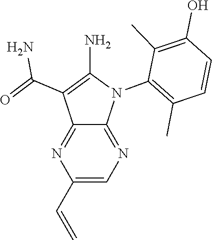 [SMILES: C=Cc1cnc2c(n1)c(C(N)=O)c(N)n2-c1c(C)ccc(O)c1C] | 259 |
| 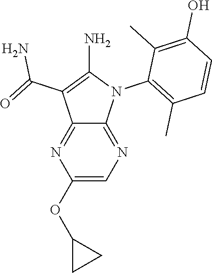 [SMILES: Cc1ccc(O)c(C)c1-n1c(N)c(C(N)=O)c2nc(OC3CC3)cnc21] | 260 |
| 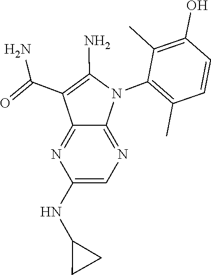 [SMILES: Cc1ccc(O)c(C)c1-n1c(N)c(C(N)=O)c2nc(NC3CC3)cnc21] | 261 |
| 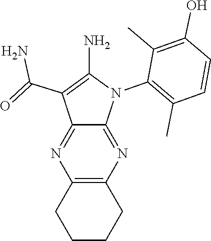 [SMILES: Cc1ccc(O)c(C)c1-n1c(N)c(C(N)=O)c2nc3c(nc21)CCCC3] | 262 |
| 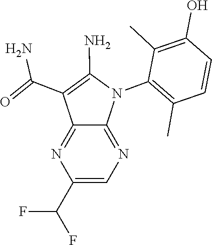 [SMILES: Cc1ccc(O)c(C)c1-n1c(N)c(C(N)=O)c2nc(C(F)F)cnc21] | 263 |
| 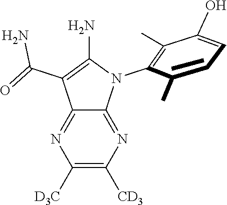 | 264 |
| 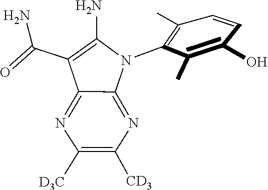 | 265 |
| 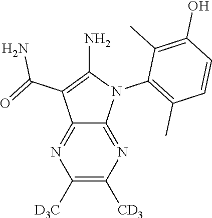 [SMILES: [2H]C([2H])([2H])c1nc2c(C(N)=O)c(N)n(-c3c(C)ccc(O)c3C)c2nc1C([2H])([2H])[2H]] | 266 |
| 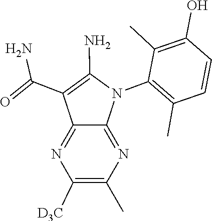 [SMILES: [2H]C([2H])([2H])c1nc2c(C(N)=O)c(N)n(-c3c(C)ccc(O)c3C)c2nc1C] | 267 |
| 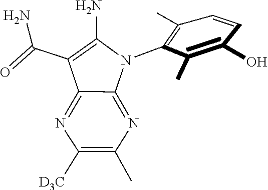 | 268 |
| 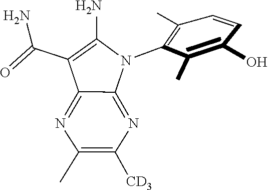 | 269 |
| 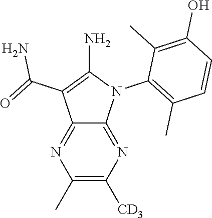 [SMILES: [2H]C([2H])([2H])c1nc2c(nc1C)c(C(N)=O)c(N)n2-c1c(C)ccc(O)c1C] | 270 |
| 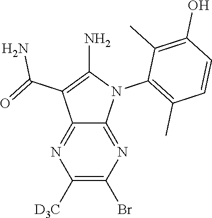 [SMILES: [2H]C([2H])([2H])c1nc2c(C(N)=O)c(N)n(-c3c(C)ccc(O)c3C)c2nc1Br] | 271 |
| 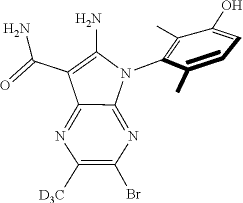 | 272 |
| 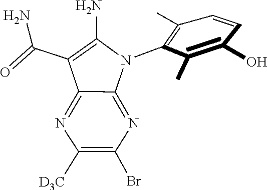 | 273 |
| 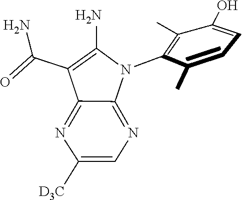 | 274 |
| 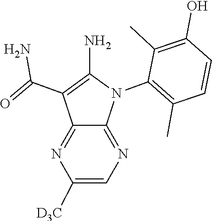 [SMILES: [2H]C([2H])([2H])c1cnc2c(n1)c(C(N)=O)c(N)n2-c1c(C)ccc(O)c1C] | 275 |
| 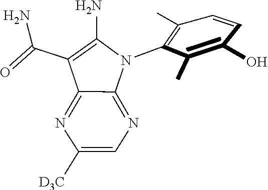 | 276 |
| 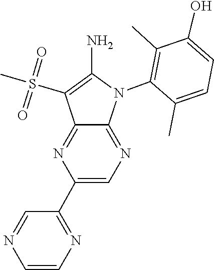 [SMILES: Cc1ccc(O)c(C)c1-n1c(N)c(S(C)(=O)=O)c2nc(-c3cnccn3)cnc21] | 278 |
| 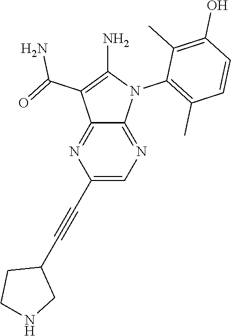 [SMILES: Cc1ccc(O)c(C)c1-n1c(N)c(C(N)=O)c2nc(C#CC3CCNC3)cnc21] | 279 |
| 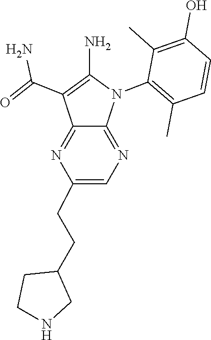 [SMILES: Cc1ccc(O)c(C)c1-n1c(N)c(C(N)=O)c2nc(CCC3CCNC3)cnc21] | 280 |
| 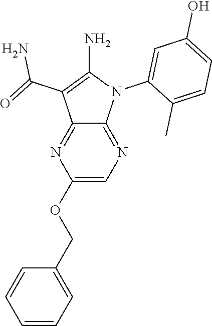 [SMILES: Cc1ccc(O)cc1-n1c(N)c(C(N)=O)c2nc(OCc3ccccc3)cnc21] | 281 |
| 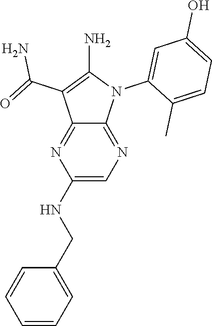 [SMILES: Cc1ccc(O)cc1-n1c(N)c(C(N)=O)c2nc(NCc3ccccc3)cnc21] | 282 |
| 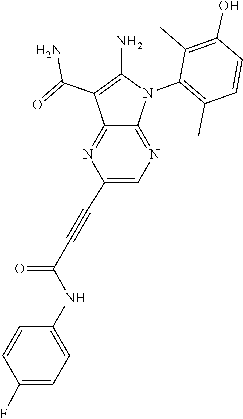 [SMILES: Cc1ccc(O)c(C)c1-n1c(N)c(C(N)=O)c2nc(C#CC(=O)Nc3ccc(F)cc3)cnc21] | 283 |
| 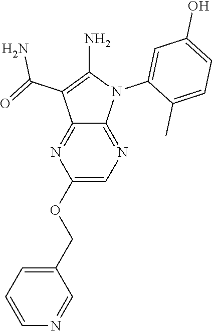 [SMILES: Cc1ccc(O)cc1-n1c(N)c(C(N)=O)c2nc(OCc3cccnc3)cnc21] | 284 |
| 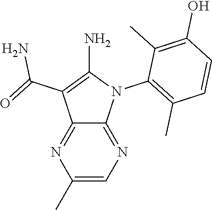 [SMILES: Cc1cnc2c(n1)c(C(N)=O)c(N)n2-c1c(C)ccc(O)c1C] | 285 |
| 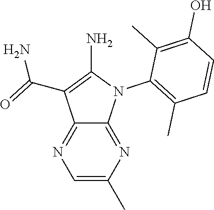 [SMILES: Cc1cnc2c(C(N)=O)c(N)n(-c3c(C)ccc(O)c3C)c2n1] | 286 |
| 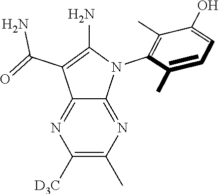 | 287 |
| 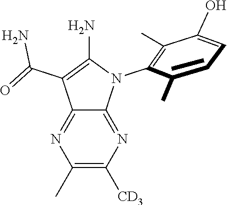 | 288 |
| 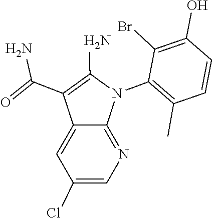 [SMILES: Cc1ccc(O)c(Br)c1-n1c(N)c(C(N)=O)c2cc(Cl)cnc21] | 289 |
| 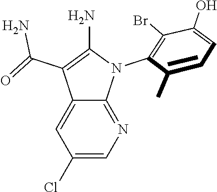 | 290 |
| 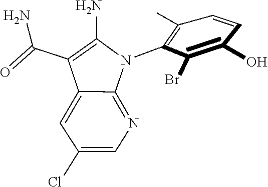 | 291 |
| 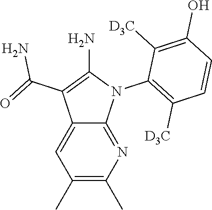 [SMILES: [2H]C([2H])([2H])c1ccc(O)c(C([2H])([2H])[2H])c1-n1c(N)c(C(N)=O)c2cc(C)c(C)nc21] | 292 |
| 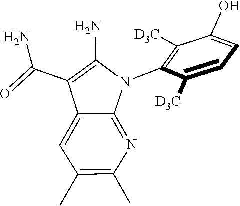 | 293 |
| 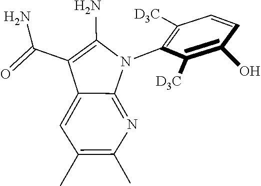 | 294 |
| 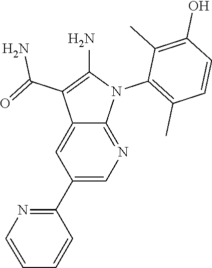 [SMILES: Cc1ccc(O)c(C)c1-n1c(N)c(C(N)=O)c2cc(-c3ccccn3)cnc21] | 295 |
| 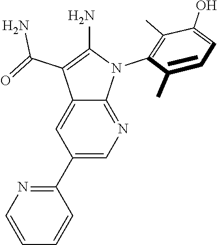 | 296 |
| 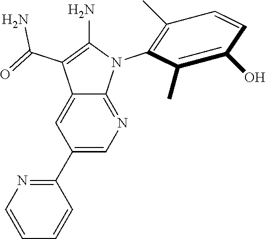 | 297 |
| 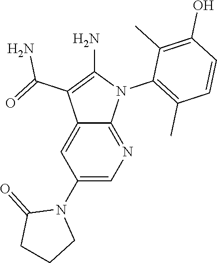 [SMILES: Cc1ccc(O)c(C)c1-n1c(N)c(C(N)=O)c2cc(N3CCCC3=O)cnc21] | 298 |
| 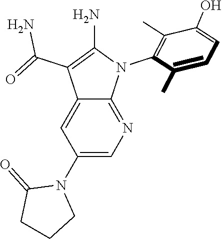 | 299 |
| 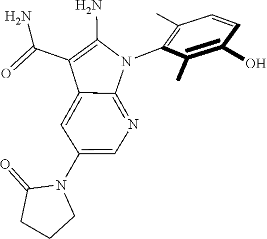 | 300 |
| 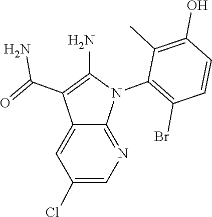 [SMILES: Cc1c(O)ccc(Br)c1-n1c(N)c(C(N)=O)c2cc(Cl)cnc21] | 301 |
| 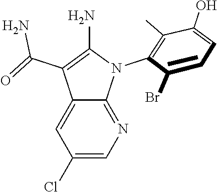 | 302 |
| 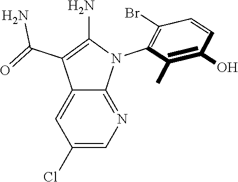 | 303 |
| 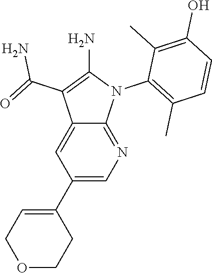 [SMILES: Cc1ccc(O)c(C)c1-n1c(N)c(C(N)=O)c2cc(C3=CCOCC3)cnc21] | 304 |
| 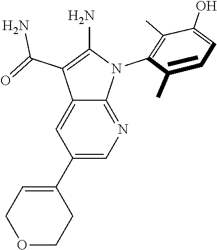 | 305 |
| 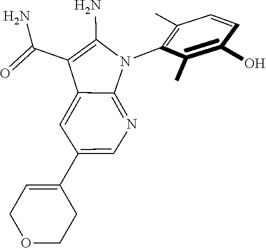 | 306 |
| 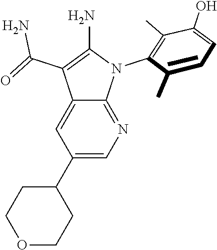 | 307 |
| 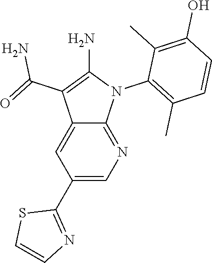 [SMILES: Cc1ccc(O)c(C)c1-n1c(N)c(C(N)=O)c2cc(-c3nccs3)cnc21] | 308 |
| 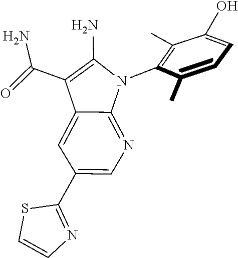 | 309 |
| 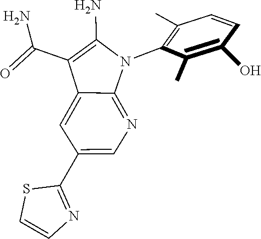 | 310 |
| 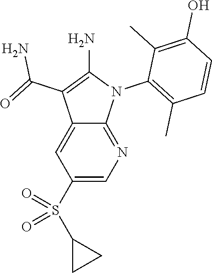 [SMILES: Cc1ccc(O)c(C)c1-n1c(N)c(C(N)=O)c2cc(S(=O)(=O)C3CC3)cnc21] | 311 |
| 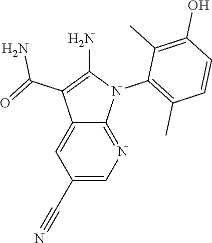 [SMILES: Cc1ccc(O)c(C)c1-n1c(N)c(C(N)=O)c2cc(C#N)cnc21] | 312 |
| 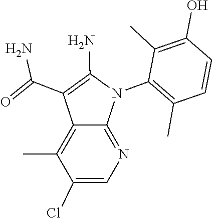 [SMILES: Cc1ccc(O)c(C)c1-n1c(N)c(C(N)=O)c2c(C)c(Cl)cnc21] | 313 |
| 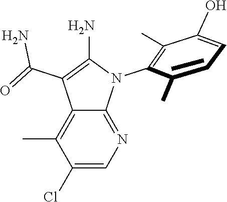 | 314 |
| 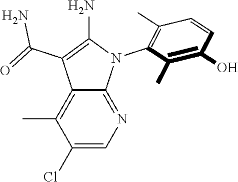 | 315 |
| 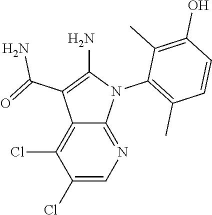 [SMILES: Cc1ccc(O)c(C)c1-n1c(N)c(C(N)=O)c2c(Cl)c(Cl)cnc21] | 316 |
| 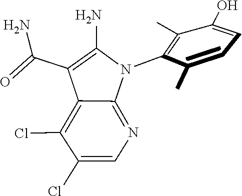 | 317 |
| 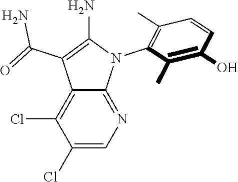 | 318 |
| 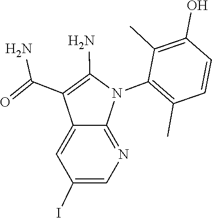 [SMILES: Cc1ccc(O)c(C)c1-n1c(N)c(C(N)=O)c2cc(I)cnc21] | 319 |
| 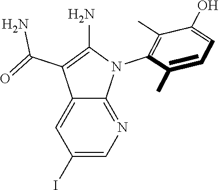 | 320 |
| 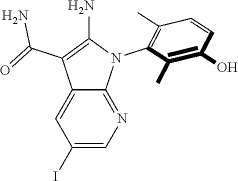 | 321 |
| 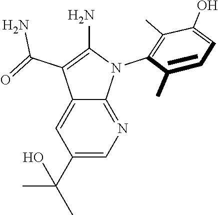 | 322 |
| 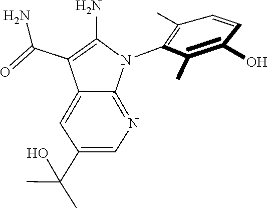 | 323 |
| 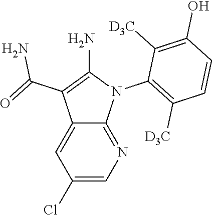 [SMILES: [2H]C([2H])([2H])c1ccc(O)c(C([2H])([2H])[2H])c1-n1c(N)c(C(N)=O)c2cc(Cl)cnc21] | 324 |
| 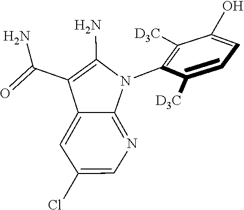 | 325 |
| 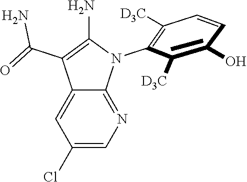 | 326 |
| 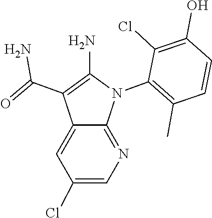 [SMILES: Cc1ccc(O)c(Cl)c1-n1c(N)c(C(N)=O)c2cc(Cl)cnc21] | 327 |
| 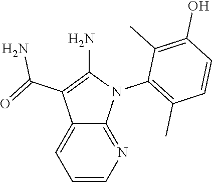 [SMILES: Cc1ccc(O)c(C)c1-n1c(N)c(C(N)=O)c2cccnc21] | 328 |

### EXAMPLES

### Example 1. Identifying New Control Loci for FISH

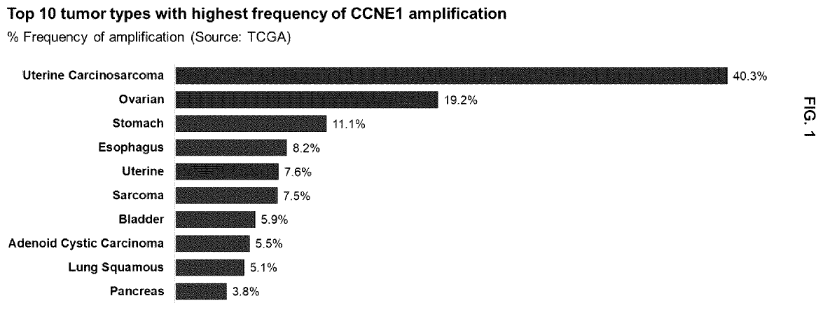

Increased gene expression is often associated with carcinoma and may be the result of a gene duplication event, chromosome (chr) duplication event, or both. For example, amplification of the cyclin E1 (CCNE1) gene is associated with many types of cancers (e.g., see FIG. 1). Standard methods for determining amplification of a gene utilize a pair of fluorescent in situ hybridization (FISH) nucleic acid probes (e.g., fluorescent probes) to quantify such cytogenetic abnormalities (e.g., Ayse et al. (2017) Mod Path., 20:297-303; Kuhn et al. (2013) Mod Path., 27:1014-1019; Karst et al. (2014) Cancer Res., 74(4):1141-1152; and Aziz et al., (2018) Gynecol Oncol., 151(2):327-336, each herein incorporated by reference). Using the CCNE1 gene as an example, the first FISH probe (e.g., the target probe) is designed to bind to all or part of the CCNE1 (e.g., see FIG. 4), and the second FISH probe (e.g., the control probe) is designed to bind to a stable control locus (e.g., a chromosomal band) on the same chromosome but remains separate and distinct from the CCNE1 loci. The ratio of the fluorescent signals produced by the two FISH probes can indicate the amplification status of the CCNE1 gene and thus inform one about the likelihood of a cancerous cell.

In this example, we identified several chromosomal bands (e.g., see Table 2) within chromosome 19 (chr19) that have substantially constant ploidy in tumor cells across targeted cancer indications (e.g., patients with ovarian cancer and uterine malignancies); therefore, these chromosomal bands can serve as a stable control locus for a FISH assay used to determine amplification of a gene of interest (e.g., a target genomic locus).

To identify these bands, we first analyzed copy number variation (i.e., ploidy) data from The Cancer Genome Atlas (TCGA). Copy number information for tumors from TCGA was downloaded from the NIH Genomic Data Commons Data Portal (portal.gdc.cancer.gov/) on Jun. 6, 2020.

Chromosomal band name and genomic location was downloaded from Ensembl's BioMart (useast.ensembl.org/info/data/biomart/index) on Oct. 16, 2020. Band level copy number was determined by intersecting the ASCAT2 allele-specific copy number segment files with the band's genomic position using the package bedr in the R working environment. A weighted average (e.g., mean) was used to calculate the ploidy of chromosomal bands where chromosome segments overlapped. This caused the ploidy status of these bands to no longer be integers. For each indication examined, we calculated the percentage of patients where the weighted average copy number of a chromosomal band was between 1.8 and 2.2 copies, which was considered diploid (i.e., un-amplified). For each indication, we ranked the chromosomal bands according to the percentage of patients with diploid status. For each band, the average rank across indications was calculated. The average rank was then used to identify the chromosomal bands that were most consistently diploid across the indications of interest (e.g., see Table 2). The top ranked (upper quartile) loci shown in Table 2 (e.g., q13.43, q13.41, q13.42, p13.3, q13.32, and q13.33) represent an expanded repertoire of control loci that may be utilized in the FISH methods provided herein to accurately determine amplification of CCNE1 in a cell and, therefore, inform one about the likelihood of a cancerous cell.

TABLE 2
Average rank of chromosome 19 bands

| | | % of patients where | | | | | | |
|---|---|---|---|---|---|---|---|---|
| | | band ploidy = 2 | | | Ploidy rank within chromosome 19 | | | |
| Chromosome | | TCGA- | TCGA- | TCGA- | TCGA- | TCGA- | TCGA- | Average |
| (Chr) | Band | OV | UCEC | UCS | OV | UCEC | UCS | rank |
| chr19 | q13.43 | 36.74 | 72.2 | 30.19 | 14 | 17 | 16.5 | 15.83 |
| chr19 | q13.41 | 39.61 | 70.9 | 24.53 | 18 | 14 | 12 | 14.67 |
| chr19 | q13.42 | 36.38 | 71.46 | 26.42 | 13 | 16 | 13.5 | 14.17 |
| chr19 | p13.3 | 28.49 | 73.88 | 50.94 | 6 | 18 | 18 | 14 |
| chr19 | q13.32 | 38.35 | 70.52 | 28.3 | 16 | 11 | 15 | 14 |
| chr19 | q13.33 | 38.71 | 71.08 | 22.64 | 17 | 15 | 9 | 13.67 |
| chr19 | q13.31 | 36.92 | 69.59 | 30.19 | 15 | 5.5 | 16.5 | 12.33 |
| chr19 | p13.13 | 29.75 | 70.34 | 26.42 | 10 | 10 | 13.5 | 11.17 |
| chr19 | q13.13 | 32.62 | 70.71 | 20.75 | 12 | 12.5 | 5.5 | 10 |
| chr19 | p13.12 | 29.75 | 70.15 | 22.64 | 10 | 8 | 9 | 9 |
| chr19 | p13.2 | 26.52 | 70.71 | 22.64 | 3 | 12.5 | 9 | 8.17 |
| chr19 | q13.2 | 27.96 | 70.15 | 22.64 | 4 | 8 | 9 | 7 |
| chr19 | p12 | 28.32 | 69.59 | 22.64 | 5 | 5.5 | 9 | 6.5 |
| chr19 | q13.12 | 29.39 | 70.15 | 16.98 | 7.5 | 8 | 4 | 6.5 |
| chr19 | p13.11 | 29.75 | 69.22 | 20.75 | 10 | 3 | 5.5 | 6.17 |
| chr19 | q13.11 | 29.39 | 69.4 | 11.32 | 7.5 | 4 | 1 | 4.17 |
| chr19 | q11 | 25.99 | 67.54 | 15.09 | 2 | 2 | 3 | 2.33 |
| chr19 | q12 | 24.01 | 67.16 | 13.21 | 1 | 1 | 2 | 1.33 |

Bands represented are relative to the Homo sapiens genome assembly NCBI36.
TCGA = The Cancer Genome Atlas;
OV = ovarian;
UCEC = uterine corpus endometrial carcinoma;
UCS = uterine carcinosarcoma

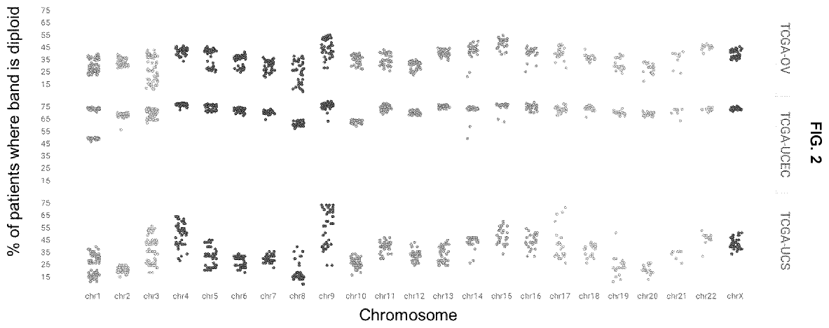

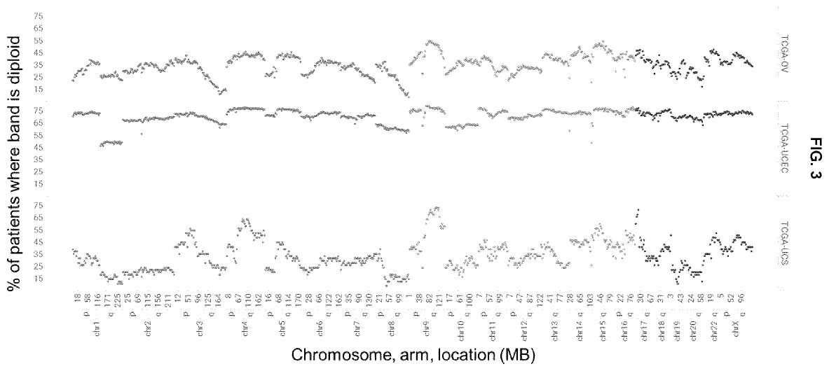

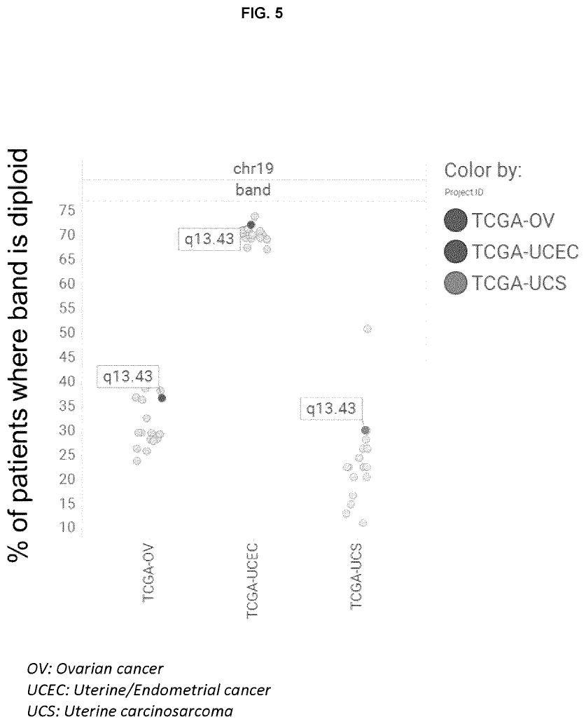

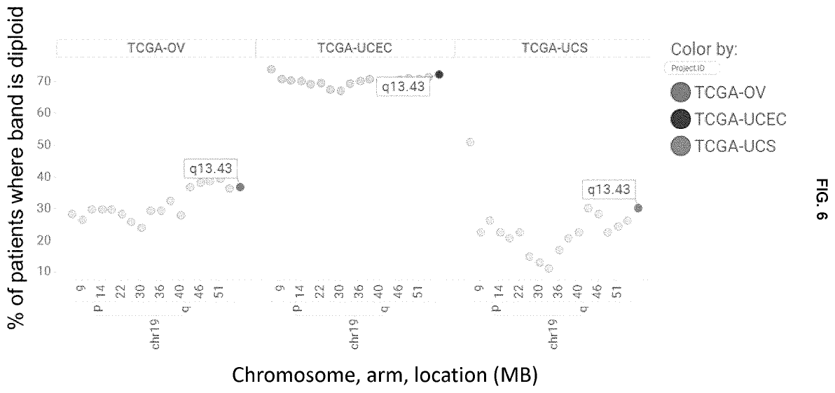

Table 2 shows metrics calculated for the following tumor indications: TCGA-OV (ovarian), TCGA-UCEC (uterine, e.g., Uterine Corpus Endometrial Carcinoma), and TCGA-UCS (uterine carcinosarcoma). The first set of metrics (e.g., “% of patients where band ploidy=2”) corresponds to the value displayed in the y-axis of the plots described in FIG. 2, FIG. 3, FIG. 5, and FIG. 6. It indicates the percentage of patients where the number of copies of a band is equal to about two (e.g., 1.8-2.2, e.g., 1.8, 1.9, 2.0, 2.1, or 2.2). The second set of metrics (e.g., “Ploidy rank within chromosome 19”) is calculated for each indication based on the percent of patients where band ploidy is about two. In other words, this metric ranks the bands such that higher percentage of diploid status is ranked with higher numbers. In cases where there are ties, the average rank is assigned to both bands. The last metric (Average rank) is calculated by averaging the ranks for each band across the indications selected. This metric is used to sort the table so that the band with the highest average rank (q13.43) is in the first row. Cross-referencing the control loci information in Table 2 with the control loci in the prior art depicted in FIG. 4 demonstrates that previously used chromosomal bands p13.3 (row 4), p13.2 (row 11), and p13.11 (row 15 and 16) would have deviations from diploid status more frequently than band q13.43.

### Example 2. Optimizing a FISH Assay for CCNE1

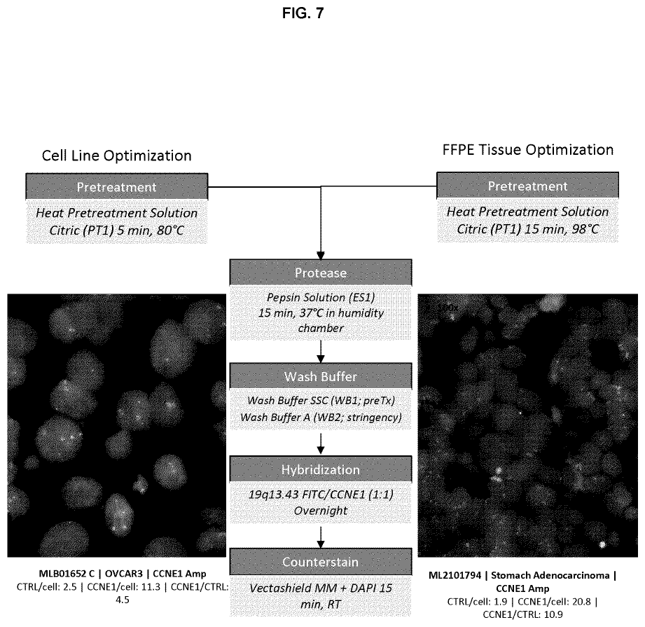

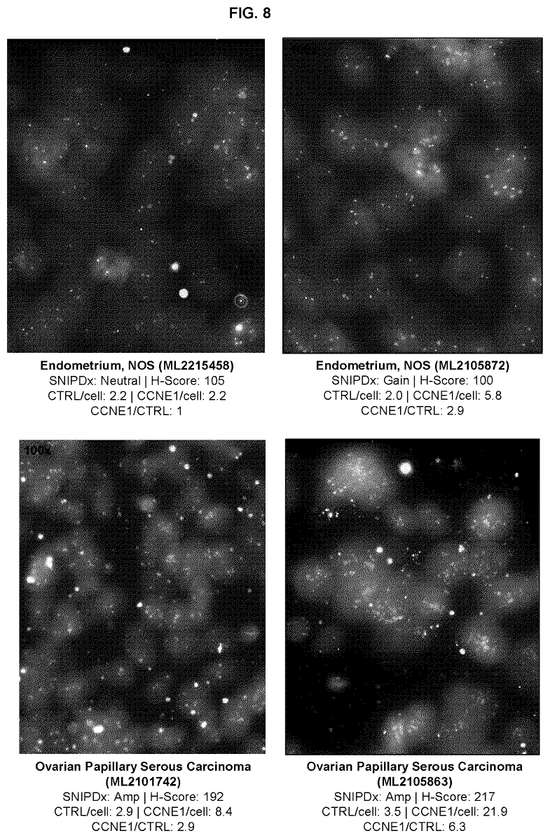

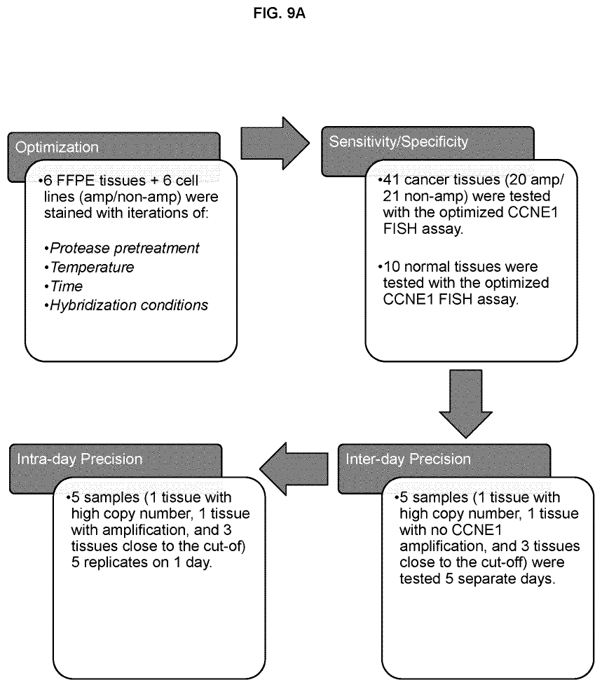

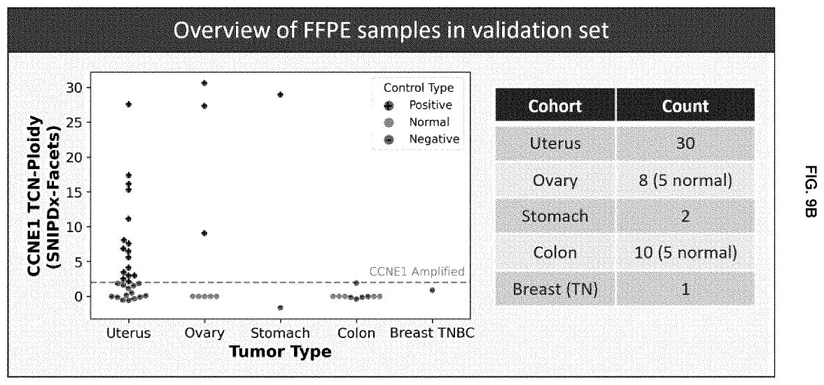

FISH was performed with a CCNE target probe on two stomach cancer tissues (CCNE1-amplified vs non-amplified) and 2 cell lines (OVCAR3 (CCNE1 amplified), MCF-7 (non-amplified)) to determine ideal assay conditions. Two hybridization times (overnight vs 2 hours) were compared. Overnight hybridization was found to provide optimal staining. Titrations of the CCNE1 probe and a sub-telomeric control probe were tested for optimal staining. A 1:1 dilution of probes was selected as the best titration. Pretreatment conditions were tested on the same tissue set by varying time and temperature. A 15-minute pretreatment step at 98° C. was selected as the optimal condition for tissue samples while a 5 min 80° C. treatment was selected for cell lines. Finally, protease treatment conditions were interrogated by varying time of treatment between 15 minutes and 25 minutes. A pepsin solution treatment for 15 min at 37° C. provided the best rate of signal vs noise ratio. The optimized CCNE1 FISH assay for staining FFPE cell line pellets and FFPE tumor tissue specimens is shown in FIG. 7. Pre-treatment conditions differ between these two types of samples, but protease and hybridization conditions are common. Representative images of cell pellet and tissue specimen staining are provided in FIG. 8. As outlined in FIG. 9A, optimal assay conditions were further tested on n=6 cancer tissues (n=2 ovarian, n=2 uterine, n=1 colon, and n=1 breast) as well as n=6 cell lines with varying CCNE1 amplification status to confirm assay performance. Enumeration of 20 cells was completed for all optimization runs. FIG. 8 shows representative images of a non-amplified endometrial tumor (FIG. 8, upper left panel), endometrial and ovarian tumors with low-level amplifications (FIG. 8, upper right and lower left panels) and an ovarian tumor with high level amplification (FIG. 8, lower right panel). Corresponding CCNE1 amplification status estimated by SNIPDx®, Cyclin E protein H-score and median of Control/CCNE1 signals per cell, estimated by FISH, are indicated below each panel.

### Example 3. Validation of FISH Assay Sensitivity, Specificity, and Precision

After staining protocol was optimized, assay sensitivity and specificity were determined as depicted in FIG. 9A. A total of 51 tissue specimens were used for validating sensitivity and specificity of the CCNE1 FISH assay, including 30 uterine cancer, tree ovarian cancer, two gastro-intestinal tumors, five colorectal cancer and one triple negative breast cancer (e.g., see right side of FIG. 9B). A total of 10 normal tissue samples from ovary (n=5) and colon (n=5) were included as well. Tumor samples were profiled using the targeted next generation sequencing assay SNiPDx® as the baseline for amplification status CCNE1 copy number calculations. FIG. 9B shows that a total of 16 specimens in the uterine cancer cohort, 3 samples from the ovarian cancer cohort and 1 sample from the gastro-intestinal cohort were considered amplified by SNiPDx®, using a threshold of

$$[ CCNE 1 Total Copy Number - Ploidy ] \geq 4$$

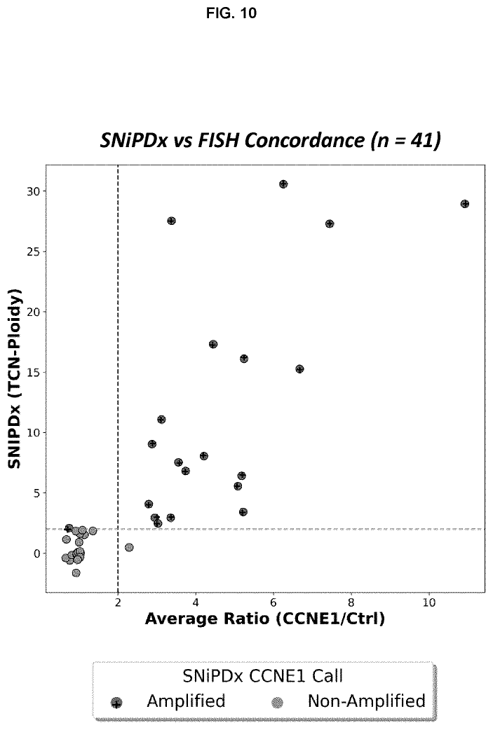

Conversely, 14 uterine tumor samples, one gastro-intestinal tumor, one triple negative breast cancer and five colorectal cancer tissues were considered non-amplified by SNIPDx®. Further, from the 41 tumor samples included in the sensitivity/specificity test, 19 were determined as amplified by both assays and 20 reported as non-amplified, leading to an overall concordance of 95.1% (39/41 samples) between both assays (e.g., see FIG. 10 and Table 3). From the discordant samples, one specimen determined as amplified by SNIPDx® was reported as non-amplified for FISH and, conversely, one tumor sample deemed as non-amplified by SNIPDx® was reported as amplified by FISH (Sensitivity: 95.0% (19/20) and Specificity: 95.2% (20/21)). Amplification calls by SNiPDx® were determined using a threshold of [CCNE1 Total Copy Number−Ploidy]≥2. The threshold for reporting amplifications by FISH is CCNE1 probe/control probe≥2.

TABLE 3
Concordance of FISH and SNiPDx ®

SNIPDx

| | | CCNE1 | CCNE1 |
|---|---|---|---|
| | | Amp | Non-Amp |
| FISH | CCNE1 Amp | 19 | 1 |
| | CCNE1 Non-Amp | 1 | 20 |

Overall concordance: 95.1% (39/41 samples)

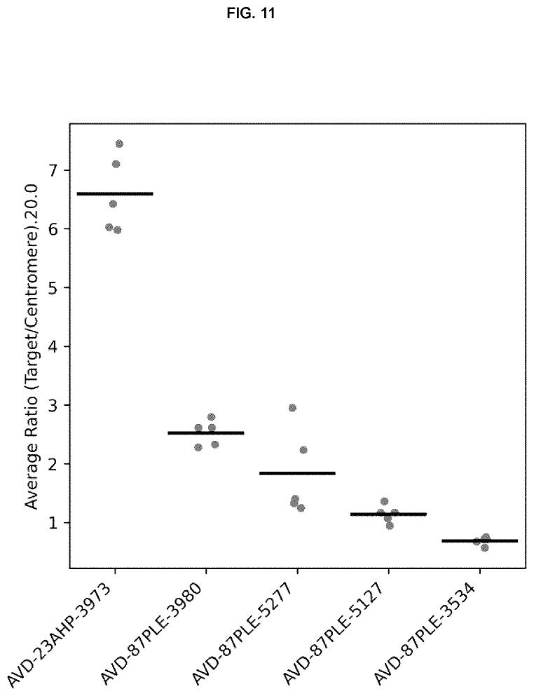

Next, as outlined in FIG. 9A, assay inter-day precision was performed using 5 tumor specimens (1 tissue with high copy number, 1 non-amplified tissue and 3 specimens with amplification levels close to the threshold criteria described above). Samples were assayed on 5 separate days. The average coefficient of variation (CV) across five samples and 5 days was 14.78% (e.g., see FIG. 11).

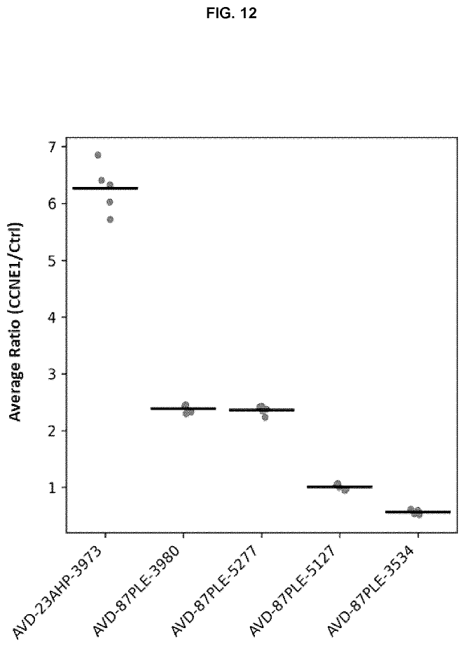

Lastly, as outlined in FIG. 9A, assay intra-day precision was determined by assaying 5 tumor samples, selected with the same criteria as on the inter-day precision experiment, as five replicates on the same day. The average coefficient of variation (CV) across five samples was 4.47% (e.g., see FIG. 12).

### Example 4. Validation of CCNE1 Copy Number with the Optimized FISH Assay

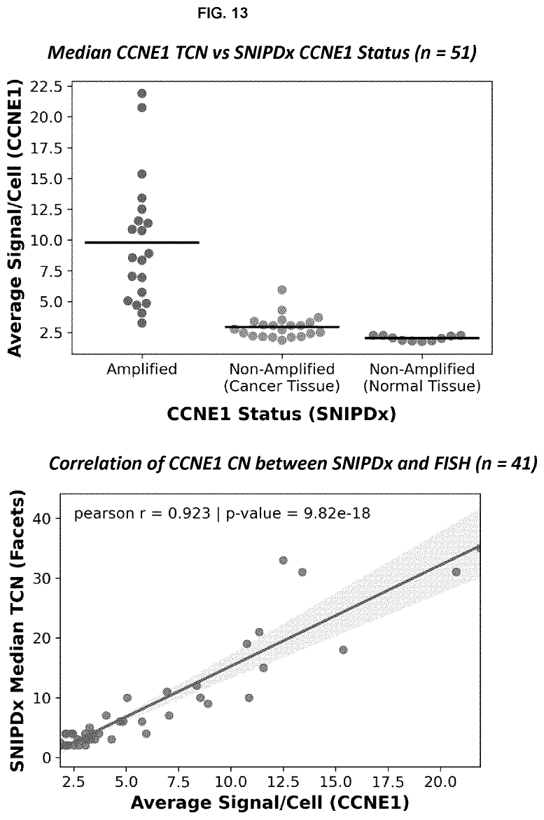

The optimized FISH assay's ability to determine CCNE1 copy number was then validated with a Next Generation Sequencing (NGS) assay, SNIPDx® (e.g., see FIG. 13). As illustrated in the upper panel of FIG. 13, the average CCNE1 signal per cell, estimated by FISH (Y axis), is significantly higher in tumor specimens deemed as CCNE1 amplified by the SNIPDx® NGS assay (median 8.7, range 3.25-21.9), compared to non-amplified tumor tissue (median 2.75, range 1.85-5.95). In normal tissue, the average number of CCNE1 copies per cell is 2, as expected for diploid and genetically stable specimens.

As illustrated in the lower panel of FIG. 13, a correlation analysis between CCNE1 total copy number estimated by FISH (X axis) and the SNIPDx® NGS assay (Y axis) shows a strong consistency between the two methodologies (Pearson ρ=0.923; m p-value=9.8 e-18).

### Example 5. Calculating Amplification in CCNE1 (Cyclin E1)

Amplification of CCNE1 in a biological sample (e.g., a tumor) can be evaluated using the methods described herein. By way of example, the sample may be a tumor sample isolated from a human subject and may be evaluated by standard fluorescent in situ hybridization (FISH) techniques utilizing using two fluorescently labeled nucleic acid probes. The first probe targets CCNE1. Such probes are standard in the field and are commercially available (e.g., Empire genomics, Inc., catalog RP11-345J21-OR). The second probe can target one the following chromosomal bands q13.43, q13.41, q13.42, p13.3, q13.32, and q13.33. FISH can be performed on the tumor sample using these two probes, thereby generating two signals. The number of signals (e.g., foci) generated by the first probe (e.g., the CCNE1 probe) is enumerated in a cell. The number of signals (e.g., foci) generated by the second probe is enumerated in a cell. Signals may be determined by fluorescent microscopy. The signal ratio of the first probe to the second probe is calculated by dividing the number of signals of the first probe by the number of signals of the second probe. It is recommended that the signal ratio of least 50 (e.g., 50, 60, 70, 80, 90, 100, 150, 200, 250, 300, or more) cells per tissue specimen is enumerated, however fewer cells per tissue specimen (e.g., 20, 25, 30, 35, 40, or 45) may be enumerated as well. The average (e.g., mean) signal ratio of all cells is then calculated. If this average signal ratio is greater than or equal to 2 (e.g., 2, 2.5, 3, 3.5, 4, 4.5, 5, 6, 7, 8, 9, 10, or more), then the CCNE1 gene in the tumor sample is considered amplified. Amplification of CCNE1 in the tumor sample is consistent with the cancerous nature of the tumor.

To calculate the total copy number (TCN) of CCNE1, divide the number of signals of the first probe by the number of cells enumerated and averaged in the tissue sample.

1. A method of identifying a stable control locus for use in a fluorescent in situ hybridization (FISH) assay, the method comprising:
(a) determining the ploidy of a plurality of chromosomal bands within a chromosome obtained from a biological sample of a subject;
(b) repeating step (a) a plurality of times, wherein the biological sample is obtained from a different subject each time;
(c) determining a weighted average ploidy of each chromosomal band, wherein a weighted average ploidy between 1.8 copies and 2.2 copies is considered diploid;
(d) determining a percentage of subjects that are considered diploid for each chromosomal band;
(e) ranking each chromosomal band, from high to low, by the percentage of subjects that are considered diploid; and
(f) selecting a chromosomal band within the upper quartile of step (e), thereby identifying the stable control locus.

2. The method of claim 1, wherein the biological sample comprises a cancer cell.

3. (canceled)

4. A method of determining if cyclin E1 (CCNE1) is amplified in a cell, the method comprising:
(a) contacting a population of cells having chromosomal DNA with:
(i) a plurality of target probes that are capable of emitting a first detectable signal and are complementary to CCNE1, and
(ii) a plurality of control probes that are capable of emitting a second detectable signal and are complementary to a CCNE1 control locus, wherein the CCNE1 control locus and CCNE1 are on the same chromosomal DNA molecule;
(b) quantifying the total number of first detectable signals and second detectable signals in a cell;
(c) determining a signal ratio in the cell of step (b);
(d) repeating step (b) and step (c) a plurality of times, wherein the cell is different each time;
(e) determining an average signal ratio of step (d); and
(f) determining amplification of CCNE1 from the average signal ratio, wherein an average signal ratio ≥2 indicates that CCNE1 is amplified.

5. The method of claim 4, wherein the plurality of target probes binds within the q12 band of chromosome (chr) 19 (chr19 q12), relative to Homo sapiens genome assembly NCBI36, or within chr19 q13.43, chr19 q13.41, chr19 q13.42, chr19 p13.3, chr19 q13.32, chr19 q13.33, chr19 q13.31, chr19 p13.13, or chr19 q13.13, relative to Homo sapiens genome assembly NCBI36.

6. (canceled)

7. The method of claim 5, wherein the plurality of control probes binds within chr19 q13.43, relative to Homo sapiens genome assembly NCBI36, or within nucleotide positions 29,644,930-29,841,126 of chr19 q13.43, relative to Homo sapiens genome assembly NCBI36.

8. (canceled)

9. A method of determining if a target genomic locus is amplified in a cell, the method comprising:
(a) contacting a population of cells having chromosomal DNA with:
(i) a plurality of target probes that are capable of emitting a first detectable signal and are complementary to the target genomic locus, and
(ii) a plurality of control probes that are capable of emitting a second detectable signal and are complementary to a stable control locus, wherein the stable control locus and the target genomic locus are on the same chromosomal DNA molecule;
(b) quantifying the total number of first detectable signals and second detectable signals in a cell;
(c) determining a signal ratio in the cell of step (b);
(d) repeating step (b) and step (c) a plurality of times, wherein the cell is different each time;
(e) determining an average signal ratio of step (d); and
(f) determining amplification of the target genomic locus from the average signal ratio, wherein an average signal ratio ≥2 indicates that the target genomic locus is amplified.

10. The method of claim 9, wherein the stable control locus is identified by:
(a) determining the ploidy of a plurality of chromosomal bands within a chromosome obtained from a biological sample of a subject;
(b) repeating step (a) a plurality of times, wherein the biological sample is obtained from a different subject each time;
(c) determining a weighted average ploidy of each chromosomal band, wherein a weighted average ploidy between 1.8 copies and 2.2 copies is considered diploid;
(d) determining a percentage of subjects that are considered diploid for each chromosomal band;
(e) ranking each chromosomal band, from high to low, by the percentage of subjects that are considered diploid; and
(f) selecting a chromosomal band within the upper quartile of step (e), thereby identifying the stable control locus.

11. The method of claim 10, wherein the biological sample comprises a cancer cell.

12. (canceled)

13. The method of claim 4, wherein the population of cells are from a human tumor sample, a human subject diagnosed with cancer, a human subject at risk of developing cancer, or a human subject suspected of having cancer.

14.-20. (canceled)

21. A method of identifying a patient at risk of having cancer or suspected of having cancer, the method comprising:
(a) obtaining a sample of cells from a subject; and
(b) performing the method of any one of claim 4,
wherein the subject is identified as a patient if the CCNE1 gene is determined to be amplified in the sample.

22. A method of treating the patient of claim 21, the method comprising administering an effective amount of an inhibitor of a membrane-associated tyrosine and threonine-specific cdc2-inhibitory kinase (Myt1).

23. The method of claim 22, wherein the Myt1 inhibitor is a compound of formula (I) 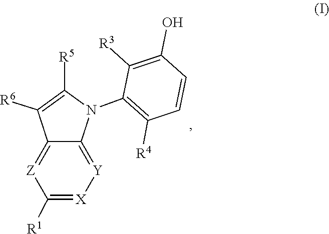
or a pharmaceutically acceptable salt thereof,
wherein
each of X, Y, and Z is independently N or CR²;
R¹ and each R² are independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B}; or R¹ combines with one R² that is vicinal to R¹ to form an optionally substituted C₃₋₆ alkylene;
each of R³ and R⁴ is independently optionally substituted C₁₋₆ alkyl or halogen;
R⁵ is H or —N(R⁷)₂;
R⁶ is —C(O)NH(R⁸), —C(O)R^{7A}, or —SO₂R^{7A};
each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;
each R^{7A} is independently optionally substituted C₁₋₆ alkyl, optionally substituted C₃₋₈ cycloalkyl, or optionally substituted C₆₋₁₀ aryl;
each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;
each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;
Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene.

24. The method of claim 23, or a pharmaceutically acceptable salt thereof, wherein the compound is enriched for the atropisomer of formula (IA):

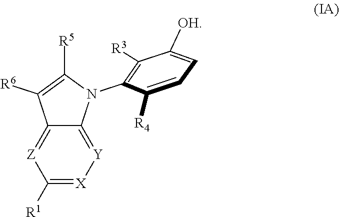

25. The method of claim 23, or a pharmaceutically acceptable salt thereof, wherein X is CR².

26. The method of claim 23, wherein the compound is of formula (II):

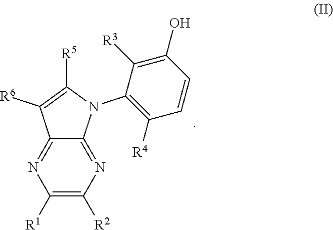

27. The method of claim 26, wherein the compound is enriched for the atropisomer of formula (IIA):

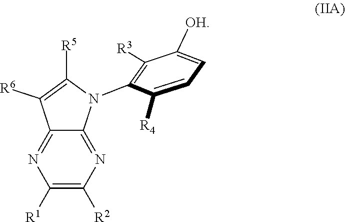

28. The method of claim 23, wherein the compound is of formula (III): 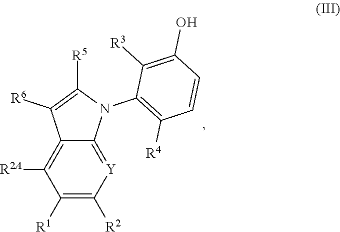
wherein R^{2A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, —N(R⁷)₂, —OR⁷, —C(O)N(R⁸)₂, —SO₂N(R⁸)₂, —SO₂R^{7A}, or -Q-R^{7B}.

29. The method of claim 28, wherein the compound is enriched for the atropisomer of formula (IIIA):

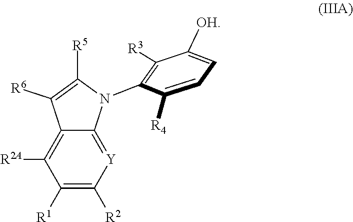

30. The method of claim 22, wherein the Myt1 inhibitor is selected from the group consisting of compounds 1-328 and pharmaceutically acceptable salts thereof.

31. The method of claim 22, wherein the Myt1 inhibitor is compound 182 or a pharmaceutically acceptable salt thereof.
